(19) United States
(12) Patent Application Publication
(10) Pub. No.: US 2003/0092174 A1
(43) Pub. Date: May 15, 2003

(54) Tissues or organs for use in xenotransplantation

(76) Inventors: Monika Liljedahl, La Jolla, CA (US); Daniela Marcantonio, San Diego, CA (US); Simon Eric Aspland, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/147,286

(22) Filed: May 14, 2002

Related U.S. Application Data
(60) Provisional application No. 60/291,394, filed on May 14, 2001.
(60) Provisional application No. 60/312,125, filed on Aug. 13, 2001.
(60) Provisional application No. 60/367,090, filed on Mar. 21, 2002.

(51) Int. Cl. C12N 5/02; A61K 31/711; C07H 21/04

(52) U.S. Cl. 435/325; 536/23.1; 514/44

(57) ABSTRACT

The present invention provides cells, tissues or organs for use in cell therapy or xenotransplantation in which at least one gene comprising an antigenic determinant recognized by a recipient organism has been disrupted. The present invention also includes methods of administering such cells and transplanting such tissues or organs in which genes encoding antigenic determinants recognized by the recipient organism have been disrupted.

### RELATED APPLICATIONS

This application claims priority to U.S. Provisional Application Serial No. 60/291,394, Filed May 14, 2001, U.S. Provisional Application Serial No. 60/312,125, Filed Aug. 13, 2001, and U.S. Provisional Application Serial No. 60/367090, Filed Mar. 21, 2002; each of which is entitled TISSUES OR ORGANS FOR USE IN XENOTRANSPLANTATION. The disclosures of each of the foregoing Provisional Applications are incorporated by reference herein in their entireties.

### BACKGROUND OF THE INVENTION

Organ or tissue failure may result from a variety of causes including genetic defects or damage induced by infectious disease, cancerous processes, toxic substances, autoimmune disorders (e.g., type 1 diabetes mellitus) as well as chronic medical conditions. Patients suffering from organ or tissue failure have very limited treatment options.

Transplantation procedures are now relatively safe procedures, but the demand for organs or tissues is by far exceeding the supply. Only about 5% of patients waiting for an organ or tissue transplant will receive one. Thus, at the present time, allotransplantation (i.e., donation of organs or tissues from other human beings) is not available for many individuals in need of an organ or tissue transplant. Accordingly, there is an enormous need to provide organs or tissues to patients who have no other treatment for their condition but a new organ or tissue.

One approach to this problem is xenotransplantation, where organs or tissues from another species are transplanted into the recipient. One advantage of xenotransplantation is that a large number of donor organs or tissues may be obtained from animals. Xenotransplantation has been successfully used to transplant encapsulated pig pancreatic islets to human subjects suffering from type 1 diabetes, thereby controlling the disease and eliminating the need for daily insulin injections (Murakami et al., 2000, Transplantation 70(8):1143-1148). However, in the foregoing procedure, the transplanted tissue was protected from rejection by the host by means of a semipermeable barrier that permitted access to nutrients and the release of insulin, but did not allow antibodies and immune cells from the host to access the transplanted islet cells.

It is desirable to provide organs or tissues which can be used in xenotransplantation procedures without the necessity of encapsulating them in artificial materials in order to shelter them from the antibodies and immune cells of the recipient organism. However, prior to the present invention, organs originating from other organisms were rejected by the recipient due to the presence of molecules recognized by the immune system of the recipient on the transplanted tissue or organ.

In particular, prior pig to primate transplantation attempts have been unsuccessful. The rejection process can be divided into four different events: hyperacute rejection, acute rejection, vascular rejection and delayed cellular rejection. The first two steps of rejection, hyperacute rejection and acute rejection, occurred within minutes to hours of the transplant and were caused by natural antibodies residing in the recipient organism. The most prominent target of the recipient's immune response was a specific carbohydrate [Gal-α (1-3)-Gal] or gal-3 located at the end of glycolipids and glycoproteins present on the cell surface of pig endothelium. Antibody recognition of gal-3 triggers the complement cascade and leads to massive cellular destruction, organ destruction and finally rejection. Knock out mutations in the GGTA1 gene, which encodes a protein responsible for gal-3 production, have been constructed in mice by homologous recombination (Tearle et al., 1996, Transplantation 61(1):13-19) but such knockouts have not been constructed in organisms amenable to use in xenotransplantation procedures for humans.

To overcome this problem, several approaches have been taken. One is to overexpress a carbohydrate-modifying enzyme, α (alpha)-1.3 fucosyl transferase, that is used to compete with gal-3 production in the primate (Koike et al., 1996, Xenotransplantation 3:81-86) and another is to massively deplete the host of its natural antibodies against gal-3, either specifically or more generally (Rydberg et al., 1995, Xenotransplantation 2:253-263; Sachs et al., 1995, Xenotransplantation 2:234-239). This approach reduced the first two steps of rejection, but the resulting lack of protective natural antibodies in the recipient puts the recipient at risk of infection by enterobacteria.

A third approach was to overexpress inhibitors of the complement system. To reduce the antibody response, decay accelerating factors (DAF, CD55), membrane cofactor protein (MCP and DC46) and CD49 (Platt et al., 1996, Transplantation Reviews 10:69-77) have been overexpressed. While this approach reduced the acute rejection process, the consequences of permanently suppressing the complement system may pose health risks for the recipient.

### FIELD OF THE INVENTION

The present invention relates to cells, tissues or organs for use in xenotransplantation procedures. The cells, tissues or organs have a disruption in at least one gene encoding a polypeptide comprising an antigenic determinant recognized by the recipient organism such that recognition of the antigenic determinant by the recipient organism is reduced or eliminated.

### SUMMARY OF THE INVENTION

Embodiments of the present invention relate to genetically engineered cells, tissues and organs available in unlimited supply which have a reduced level of immunogenicity in the recipient and which can be safely transplanted across species. If desired, immunosupressant medications, such as cyclosporins, may be administered to recipients of the transplanted organs or tissues, but the amount of such medications may be reduced in light of the reduced level of immunogenicity of the donor tissues or organs. This can be desirable due to the undesireable side effects of immunosuppressants.

Transplantation of the tissues or organs of the present invention may also reduce the amount of medication required to induce a state of immunotolerance in the host. In the method described here, cells that have been modified to have reduced immunogenicity in the recipient organism are used to produce tissues or organs for use in xenotransplantation procedures.

In addition, some medical conditions may be ameliorated or eliminated by directly administering cells, which need not be associated with one another in a tissue or organ, to a recipient in need of a beneficial factor produced by the cells. In such contexts, it is desirable to administer cells which will not be rejected by the recipient. The present invention provides cells useful in such procedures.

Some embodiments of the present invention are described below. However, it will be appreciated that the scope of the present invention is defined solely by the appended claims. Accordingly, other embodiments which will be apparent to those of ordinary skill in the art in view of the disclosure herein are also within the scope of this invention.

One embodiment of the present invention is a method of obtaining a tissue or organ having a reduced level of rejection in a recipient organism comprising obtaining a cell from a donor organism, disrupting at least one gene in said cell which encodes a polypeptide comprising an antigenic determinant recognized by said recipient organism, thereby generating a modified cell, generating an organism from said modified cell, wherein said organism comprises cells in which said at least one gene has been disrupted, and obtaining tissues or organs from said organism. In some aspects of this embodiment, a plurality of genes encoding polypeptides comprising an antigenic determinant recognized by said recipient organism are disrupted. For example, in some aspects of this embodiment, at least two, at least 4, at least 5, at least 10, at least 15, at least 20, at least 25, at least 35, at least 40 or more than 40 genes encoding polypeptides comprising antigenic determinants recognized by said recipient organism may be disrupted. Alternatively, in some aspects of this embodiment, substantially all of the genes encoding polypeptides comprising an antigenic determinant recognized by said recipient organism may be disrupted. In some aspects of this embodiment, the cell may be from an organism selected from the group consisting of mammals, marsupials, teleost fish, avians, and the like. Examples include non-human primates, sheep, goats, cows, chickens and pigs. In some aspects of this embodiment, the cell may be a pig cell. In other aspects of this embodiment, the pig cell may be selected from the group consisting of primary pig skin fibroblasts, pig granulosa cells, pig stem cells, pig germ cells, and primary pig fetal fibroblasts. In additional aspects of this embodiment, the at least one gene is disrupted by replacing both chromosomal copies of said gene with a homologous sequence comprising a stop codon in the open reading frame which encodes said polypeptide. In some aspects of this embodiment, the method further comprises identifying at least one gene in said cell from said donor organism which encodes a polypeptide comprising an antigenic determinant recognized by said recipient organism by determining whether said polypeptide is recognized by sera from said recipient organism prior to disrupting said at least one gene. In some aspects of this embodiment, the recipient organism is a human being. In some aspects of this embodiment, the organ, tissue, or cells is selected from the group consisting of kidney, liver, pancreas, heart, lung, intestine, heart valve, cornea, or peripheral blood cells.

Embodiments of the invention are useful for treating various conditions, including those of the heart, liver, pancrease, kidney, lung, cell transplants, and other miscellaneous conditions. Non limiting examples are provided below. Examples of heart conditions include heart transplantations, Ischemic cardiomyopathy (coronary artery disease) and Idiopathic dilated cardiomyopathy, congenital heart disease, valvular heart disease, restrictive/obstructive cardiomyopathy, Anthracycline toxicity. Liver related conditions include liver transplantations, slow-growing primary hepatocellular carcinoma, liver chirrhosis, (chronic active hepatitis of cryptogenic variety, biliary cirrhosis, (a1-antitrypsin deficiency, glycogen storage disorders, galactosemia, Wilson's disease, hypercholesterolemia, hyperoxaluri, hemochromatosis), acute liver failure (hepatitis A and B, non A non B hepatitis, paracetamol or other drug induced hepatoxicities) Biliary atresia. Also included are pancreatic transplantations, diabetes, chronic pancreatitis, pancreatic carcinoma, and the like. Further, kidney conditions include kidney transplantations, primary glomerulonephritis, renal manifestations in systemic diseases (renovascular diseases, diabetes, SLE, Rheumatoid Atreritis) pyelonephritis due to infections, toxic nephropathy, and the like. Lung related conditions include lung transplantations usually in combination with heart transplant in cardiovascular disease, double-lung emphysema, cystic fibrosis, primary pulmonary hypertension, pulmonary fibrosis, and the like. Also included are cell transplantations, Alzheimer's disease, diabetes, spinal cord injury, stroke, Parkinson's disease, and miscellaneous conditions such as, cataracts, variant Creutzfeldt-Jacob disease, and so on.

Another embodiment of the present invention is a recombinant cell or a genetically engineered cell in which at least one gene encoding a polypeptide comprising an antigenic determinant which is recognized by a desired recipient organism has been disrupted. In some aspects of this embodiment, both chromosomal copies of said at least one gene have been disrupted. In some aspects of this embodiment, a plurality of genes encoding polypeptides comprising antigenic determinants recognized by a desired recipient organism have been disrupted. In some aspects of this embodiment, at least two, at least 4, at least 5, at least 10, at least 15, at least 20, at least 25, at least 35, at least 40 or more than 40 genes encoding polypeptides comprising antigenic determinants recognized by the recipient organism have been disrupted. In some aspects of this embodiment, substantially all of the genes encoding polypeptides comprising antigenic determinants recognized by the recipient organism have been disrupted. In some aspects of this embodiment, the cell is from an organism selected from the group consisting of mammals, marsupials, teleost fish, avians, and the like. Examples include non-human primates, sheep, goats, cows, chickens and pigs. In some embodiments of the present invention, the cell is a pig cell. For example, the pig cell may be selected from the group consisting of primary pig skin fibroblasts, pig granulosa cells, pig stem cells, pig germ cells, primary pig fetal fibroblasts and oocytes. In some aspects of this embodiment, at least one gene encoding a polypeptide comprising an antigenic determinant which is recognized by human beings has been disrupted.

Another embodiment of the present invention is a recombinant nucleic acid comprising a 5′ region homologous to a portion of a gene encoding a polypeptide comprising an antigenic determinant recognized by a desired recipient organism, a 3′ region homologous to a portion of a gene encoding a polypeptide comprising an antigenic determinant recognized by said desired recipient organism, and at least a portion of the coding sequence of said gene disposed between said 5′ region and said 3′ region, said at least a portion of the coding sequence containing an alteration therein which prevents the synthesis of the complete polypeptide comprising an antigenic determinant recognized by said desired recipient organism. In some aspects of this embodiment, the alteration comprises a stop codon. In some aspects of this embodiment, the alteration comprises a deletion. In some aspects of this embodiment, the recombinant nucleic acid further comprises at least one nucleic acid encoding a detectable polypeptide, said at least one nucleic acid being operably linked to a promoter. In some aspects of this embodiment, the detectable polypeptide is selected from the group consisting of CD8 and green fluorescent protein. In some aspects of this embodiment, the recombinant nucleic acid comprises a nucleic acid encoding CD8 operably linked to a promoter and a nucleic acid encoding green fluorescent protein operably linked to a promoter. In some aspects of this embodiment, at least one nucleic acid encoding a detectable polypeptide is flanked by a site which facilitates excision of the nucleic acid encoding the detectable marker. In some aspects of this embodiment, the site which enables excision can be a LoxP site , an Frt site, and the like for example.

Another embodiment of the present invention is a genetically modified organism generated from any of the recombinant cells described above. In some aspects of this embodiment, the genetically modified organism is a pig.

Another aspect of the present invention is a method for performing an organ or tissue transplant comprising generating a modified cell in which at least one gene which encodes a polypeptide comprising an antigenic determinant recognized by a desired recipient organism has been disrupted, generating an organism from said modified cell, wherein said organism comprises cells in which said at least one gene has been disrupted, obtaining tissues or organs from said organism, and transplanting said tissues or organs into said recipient organism. In some aspects of this embodiment, the step of generating a modified cell comprises generating a modified cell in which at least two, at least 4, at least 5, at least 10, at least 15, at least 20, at least 25, at least 35, at least 40 or more than 40 genes encoding polypeptides comprising antigenic determinants recognized by the recipient organism have been disrupted. In some aspects of this embodiment, the step of generating a modified cell comprises generating a modified cell in which substantially all of the genes encoding polypeptides comprising antigenic determinants recognized by the recipient organism have been disrupted.

Another embodiment of the present invention is a method of disrupting a gene which encodes a polypeptide comprising an antigenic determinant recognized by a desired recipient organism comprising introducing a nucleic acid comprising a sequence homologous to at least a portion of the coding region of said gene into a cell, wherein said homologous sequence comprises a disruption in said coding region which prevents said cell from expressing the full length polypeptide normally encoded by said coding region and replacing at least one chromosomal copy of said gene with said homologous sequence comprising said disruption in said coding region.

Another embodiment of the present invention is a method of identifying a gene from a donor organism which encodes a polypeptide comprising an antigenic determinant recognized by a recipient organism comprising obtaining a cDNA library comprising a plurality of cDNAs encoding polypeptides from said donor organism, expressing said polypeptides in host cells, contacting said host cells with sera from said recipient organism and identifying host cells which express polypeptides recognized by antibodies in said sera.

Another embodiment of the present invention is a method of making a tissue or organ comprising obtaining any of the recombinant cells described above and allowing said recombinant cells to grow on a scaffold.

Another embodiment of the present invention is a method for providing a beneficial factor to a recipient organism comprising obtaining any of the recombinant cells described above wherein said recombinant cells produce said beneficial factor and administering said recombinant cells to said recipient organism. This beneficial factor could have a medicinal effect or prevent rejection of the organ, tissue or cell. In some aspects of this embodiment, the recombinant cells are not associated with one another in a tissue or organ. In some aspects of this embodiment, the cells have been genetically engineered to produce said beneficial factor. In some aspects of this embodiment, the cells are derived from pluripotent stem cells which were induced to differentiate into a desired cell type. In some aspects of this embodiment, the cells are obtained from a genetically modified animal. In some aspects of this embodiment, the cells are tissue culture or primary cells. In some aspects of this embodiment, the cells are selected from the group consisting of muscle cells, heart muscle cells, bone cells, islet cells, skin cells, nerve cells, and endothelial cells. In some aspects of this embodiment, the recipient organism is suffering from a condition selected from the group consisting of a spinal cord injury, stroke, bums, heart disease, osteoarthritis or rheumatoid arthritis, and diabetes. In some aspects of this embodiment, the cells produce a factor whose absence or production at insufficient levels has caused a disease in the recipient organism. In some aspects of this embodiment, the cells produce a factor which inhibits the activity or reduces the amount of a nucleic acid or polypeptide whose production at abnormally high levels has caused a disease in the recipient organism.

Still further embodiments relate to genetically engineered cells in which at least one gene encoding a polypeptide comprising an antigenic determinant which is recognized by a desired recipient organism or at least one gene which encodes a protein associated with the synthesis of a molecule comprising an antigenic determinant recognized by the desired recipient organism has been disrupted. Preferably, both chromosomal copies of the at least one gene have been disrupted. Further, in some embodiments at least one gene encoding a polypeptide that includes an antigenic determinant which is recognized by human beings has been disrupted. Also, in embodiments a plurality of genes encoding polypeptides that include antigenic determinants recognized by a desired recipient organism have been disrupted. In preferred embodiments at least two, at least 4, at least 5, at least 10, at least 15, at least 20, at least 25, at least 35, at least 40, more than 40 genes, and the like, encoding polypeptides comprising antigenic determinants recognized by the recipient organism have been disrupted. In other embodiments substantially all of the genes encoding polypeptides that include antigenic determinants recognized by the recipient organism have been disrupted. The cell can be from an organism, including for example, the following organisms, a mammal, a marsupial, a teleost fish, an avian, and the like. The mammal can be, for example, a non-human primate, a sheep, a goat, a cow, and the like. The avian can be a chicken, for example. Further, preferably the cell can be from a pig, and for example, the cell can be primary pig skin fibroblasts, pig granulosa cells, pig stem cells, pig germ cells, pig peripheral blood cells, pig hematopoetic stem cells, primary pig fetal fibroblasts, and the like. The at least one gene can be one that has been disrupted by replacing at least one chromosomal copy of the gene with a homologous sequence that includes a stop codon in the open reading frame which encodes the polypeptide, with a homologous sequence that includes a stop codon in all three reading frames, or with a homologous sequence comprising a deletion, for example. The at least one gene may have been disrupted by replacing at least one chromosomal copy of the gene with a non-homologous replacement nucleotide sequence flanked by nucleotide sequences homologous to a genomic sequence in which homologous recombination is desired. For example, the replacement nucleotide sequence comprises a gene encoding a marker or a gene encoding a polypeptide from the desired recipient organism. The gene encoding a polypeptide from the desired recipient organism can include a gene encoding a major histocompatability complex (MHC) Protein. The desired recipient organism can be, for example, a human being. The at least one gene can be a gene other than the GGTA1 gene. The gene can encode a polypeptide that includes an antigenic determinant or a polypeptide associated with the synthesis or modification of an antigenic determinant. The antigenic determinant can include a polypeptide, a carbohydrate, a lipid, a combination of any of the aforementioned, and the like.

Other embodiments relate to recombinant nucleic acids that include a 5′ region homologous to a portion of a gene responsible for the production of an antigenic determinant recognized by a desired recipient organism or a 5′ region homologous to a portion of a gene encoding a polypeptide associated with the synthesis of a molecule comprising an antigenic determinant recognized by the desired organism, a 3′ region homologous to a portion of a gene responsible for the production of an antigenic determinant recognized by the desired recipient organism or a 3′ region homologous to a portion of a gene encoding a polypeptide associated with the synthesis of a molecule comprising an antigenic determinant recognized by the desired organism, and a nucleotide sequence which prevents the synthesis of an antigenic determinant recognized by the desired recipient organism, the nucleotide sequence being disposed between the 5′ region and the 3′ region. The at least a portion of the nucleotide sequence which prevents the synthesis of an antigenic determinant recognized by the desired recipient organism can be disposed between the 5′ region and the 3′ region, the at least a portion containing an alteration therein which prevents the synthesis of an antigenic determinant recognized by the desired recipient organism. The alteration can include at least one deletion. The alteration can include, for example, a stop codon in the open reading frame which encodes a polypeptide that includes an antigenic determinant recognized by the desired recipient organism, a nucleotide sequence containing a stop codon in all three reading frames, or a gene encoding a marker or a gene encoding a polypeptide from the desired recipient organism. The gene encoding a polypeptide from the desired recipient organism can include a gene encoding an MHC protein. The nucleotide sequence which prevents the synthesis of an antigenic determinant recognized by the desired recipient organism can include a positive marker indicative of integration somewhere in the genome and a negative marker indicative of random integration in the genome. The positive marker can be flanked by nucleotide sequences homologous to the genomic region in which integration via homologous recombination is desired. The nucleotide sequence which prevents the synthesis of an antigenic determinant recognized by the desired recipient organism can include a promoterless marker gene flanked by nucleotide sequences which puts the marker gene under the control of the promoter which directs transcription of the gene encoding a polypeptide that includes an antigenic determinant recognized by a desired recipient organism if homologous recombination occurs. The nucleotide sequence which prevents the synthesis of an antigenic determinant recognized by the desired recipient organism can include a portion of a gene encoding a nonfunctional portion of a marker protein, the portion of the gene encoding a nonfunctional portion of a marker protein being flanked by nucleotide sequences homologous to the desired integration site. The recombinant nucleic acid sequence can further include at least one nucleic acid encoding a detectable polypeptide, the at least one nucleic acid being operably linked to a promoter. The recombinant nucleic acid can include a nucleic acid encoding CD8 operably linked to a promoter and a nucleic acid encoding green fluorescent protein operably linked to a promoter. The detectable polypeptide can be, for example, CD8, green fluorescent protein (GFP), Red fluorescent protein, Flag tag, HA tag, c-myc, GST, mbp, polyhistidine, and the like. Further, in embodiments, at least one nucleic acid encoding a detectable polypeptide can be flanked by a site which enables excision of the nucleic acid encoding a detectable polypeptide. The site which enables subsequent removal of a non-homologous sequence can be, for example, a Lox P site, an Frt site, and the like. The gene responsible for the production of an antigenic determinant can be a gene other than the GGTA1 gene. The gene can be responsible for the production of an antigenic determinant which may be a polypeptide, a carbohydrate, a lipid, or the like, or which results from the modification of a polypeptide, carbohydrate or lipid, for example.

Further embodiments relate to methods of disrupting a gene encodes a polypeptide responsible for the production of an antigenic determinant recognized by a desired recipient organism. The methods can include introducing a nucleic acid that includes a sequence homologous to at least a portion of the coding region of the gene into a cell, wherein the homologous sequence comprises a disruption in the coding region which prevents the cell from expressing the full length polypeptide normally encoded by the, coding region; and replacing at least one chromosomal copy of the gene with the homologous sequence that includes the disruption in the coding region. The method can further include enhancing the rate of recombination by introducing a double stranded break in the nucleic acid in a region in the vicinity of the gene encoding a polypeptide comprising the antigenic determinant. The double stranded break can be introduced using at least one zinc finger endonuclease domain. The disruption in the coding region can include, for example, at least one stop codon in one open reading frame encoding the polypeptide, or a nucleotide sequence containing a stop codon in all three reading frames. The gene which encodes a polypeptide that includes an antigenic determinant recognized by a desired recipient organism can be a gene other than the GGTA1 gene.

Still further embodiments relate to methods of identifying an antigenic determinant from a donor organism which is recognized by a recipient organism. The methods can include obtaining a screening composition comprising a plurality of molecules from the donor organism; contacting the plurality of molecules with naturally occurring immunoglobulin family proteins; and identifying an antigenic determinant that is detected by the naturally occurring immunoglobulin family proteins. The screening composition can include, for example, a plurality of molecules isolated from the surface of cells from the donor organism. The molecule can be, for example, a polypeptide, a lipid, a carbohydrate, a molecule comprising any combination of the foregoing molecules, and the like. The naturally occurring immunoglobulin family proteins can include, for example, immune sera from the recipient organism, a polyclonal immunoglobulin population derived from the recipient organism, and the like.

Embodiments relate to methods of identifying a gene responsible for the production of an antigenic determinant from a donor organism that is recognized by a recipient organism. The methods can include obtaining a plurality of nucleic acids encoding a plurality polypeptides from the donor organism and expressing the plurality of polypeptides; contacting the plurality of polypeptides with naturally occurring immunoglobulin family proteins present on the surface of or obtained from natural killer cells or T cells from the recipient organism; and identifying cells recognized by the naturally occurring immunoglobulin family proteins, whereby the cells include a gene from a donor organism which encodes a polypeptide that includes an antigenic determinant recognized by the recipient organism or a gene from the donor organism which encodes a polypeptide associated with the synthesis of a molecule that includes an antigenic determinant recognized by the recipient organism.

Other embodiments relate to methods of identifying a gene from a donor organism which encodes a polypeptide that includes an antigenic determinant recognized by a recipient organism or a gene from the donor organism which encodes a polypeptide associated with the synthesis of a molecule that includes an antigenic determinant recognized by the recipient organism. The methods can include obtaining a cDNA library comprising a plurality of genes encoding polypeptides from the donor organism; expressing the polypeptides in host cells; contacting the host cells with naturally occurring immunoglobulin family proteins which detect antigenic determinants recognized by the recipient organism; and identifying a host cell which expresses a polypeptide recognized by the naturally occurring Immunoglobulin family proteins. The naturally occurring immunoglobulin family proteins derived from the recipient organism can include, for example, immune sera, wherein the expressed polypeptide is recognized an antibody in the immune sera. The naturally occurring immunoglobulin family proteins derived from the recipient organism can include a polyclonal antibody population, molecules present on the surface of or obtained from natural killer cells or T cells from the recipient organism, and the like.

### BRIEF DESCRIPTION OF THE DRAWINGS

FIG. 1 illustrates the sequence of the Pst I-Bgl II fragment of the HO endonuclease (SEQ ID NO: 1).

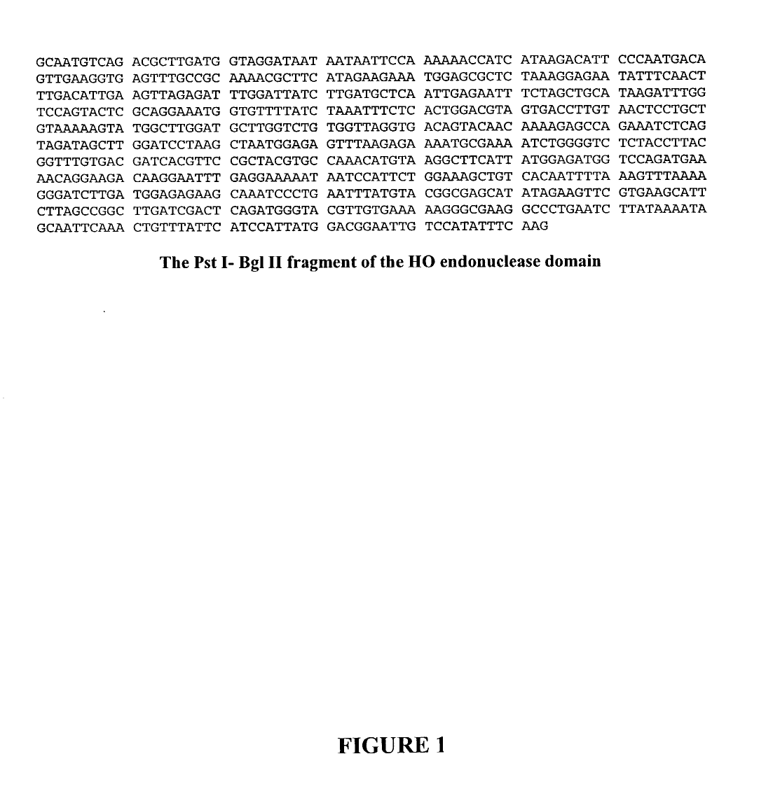

FIG. 2 illustrates a sequence for the Fok I endonuclease domain used in chimeric endonucleases (SEQ ID NO: 2).

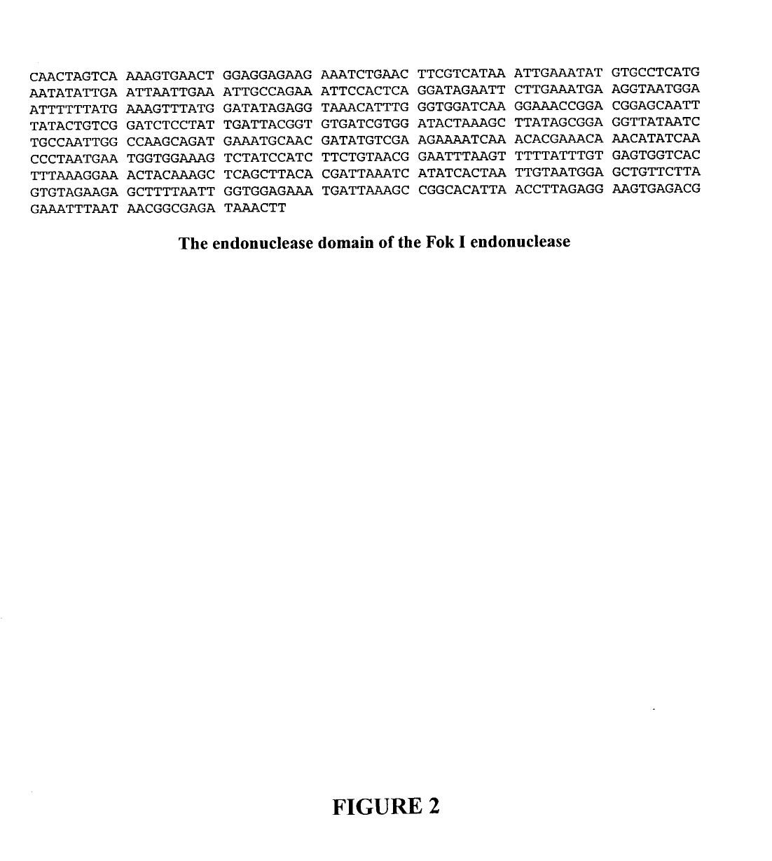

FIG. 3 illustrates exemplary zinc finger endonuclease strategies.

FIG. 4 illustrates a Sp1C framework for producing a zinc finger protein with three fingers (SEQ ID NO: 3, SEQ ID NO: 4, SEQ ID NO: 5).

FIG. 5 illustrates exemplary primers used to create a zinc finger domain with three-fingers (SEQ ID NO: 6, SEQ ID NO: 7, SEQ ID NO: 8, SEQ ID NO: 9).

FIG. 6 illustrates a method of the invention.

FIG. 7 illustrates a “Positive/Negative” homologous recombination construct.

FIG. 8 illustrates a “Gene Trap” homologous recombination construct.

FIG. 9 illustrates an “Over-lapping” homologous recombination construct.

FIG. 10 illustrates a scheme for the sequential disruption of both alleles of the exon 9 of the GGTA1 gene.

FIG. 11 illustrates a scheme for the simultaneous disruption of both alleles of the exon 9 of the GGTA1 gene.

FIG. 12 illustrates a vector and method for use in obtaining a cell, tissue or organ in which at least one gene encoding a polypeptide comprising an antigenic determinant recognized by a recipient organism has been disrupted.

FIG. 13 illustrates alternative vectors and a method for use in obtaining a cell, tissue or organ in which at least one gene encoding a polypeptide comprising an antigenic determinant recognized by a recipient organism has been disrupted.

FIG. 14 illustrates a construct strategy useful in embodiments of the invention.

### DETAILED DESCRIPTION OF THE PREFERRED EMBODIMENT

The present invention relates to methods of generating cells, tissues or organs for use in xenotransplantation or other therapies and transgenic or genetically modified organisms comprising such cells, tissues or organs. The cells, tissues or organs from the donor organism have been genetically modified to reduce or eliminate recognition of at least one antigenic determinant by the recipient organism. As used herein “donor organism” refers to the organism which provides the cells, tissues, or organs to be transplanted. As used herein “recipient organism” refers to the organism into which the cells, tissues, or organs from the donor organism are transplanted.

In a preferred embodiment, the cells, tissues or organs are suitable for xenotransplantation into a human recipient. The cells, tissues or organs may be any cell, tissue or organ suitable for use in a xenotransplantation procedure, including but not limited to kidney, liver, pancreas, heart, heart valve, lung, endothelium, brain, intestine, peripheral blood cells, or cornea. Entire organs, tissues, or portions thereof may be introduced into the recipient organism. In some embodiments, a heart valve prepared according to the present invention may be introduced into the recipient organism to treat heart valve insufficiency or stenosis. In other embodiments, cornea transplantation may be used to treat cataracts or damaged cornea.

The cells, tissues or organs may be from any organism suitable for use as a cell, organ or tissue donor for a desired recipient organism. In some embodiments, the donor organism may be a non-human primate, a mammal, a marsupial, a teleost fish, an avian, a sheep, a goat, a cow or a pig.

In one embodiment, the donor organism is a pig and the recipient organism is a human being. The organ size of minipigs is similar to that of humans, their life span is acceptable (approximately 30 years) and their litter size is large. Humans and pigs have been in close contact for thousands of years, with close contact of blood and tissue products occurring by accident and also recently by transplantation. Although retroviruses called porcine endogenous retroviruses (PERVs) which are not endogenous to humans are found in pigs, to date no studies of humans exposed to pig cells and tissues have produced any clinical or laboratory evidence of PERV spread to humans (Heneine et al., 1998, Lancet. 351(9129):695-699); Paradis et al., 1999, Science 285(5431):1236-1241; Patience et al., 1998, Curr. Opin. Immunol. 10(5):539-542). In addition, the metabolism of the pig is closely related to humans. For example, in the wild, the pigs consume a diet that derives its calories from both vegetable and animal sources.

The cells, organs or tissues therefrom have at least one gene encoding a polypeptide comprising an antigenic determinant recognized by the recipient organism disrupted or have disrupted at least one gene encoding a polypeptide associated with the synthesis of a molecule comprising an antigenic determinant recognized by the desired recipient organism. The at least one gene encoding a polypeptide associated with the synthesis of a molecule comprising an antigenic determinant can be for example, a gene encoding an enzyme that is directly or indirectly involved in the synthesis of the antigenic determinant. For example the GGTA1 gene encodes a polypeptide associated with the synthesis of gal-3, a carbohydrate which is recognized by the human immune system. The “molecule” comprising an antigenic determinant can include, for example, a polypeptide, a carbohydrate, a lipid, and the like, alone or in combination.

In some embodiments, the cells, organs or tissues have at least two, at least 4, at least 5, at least 10, at least 15, at least 20, at least 25, at least 35, at least 40 or more than 40 genes encoding polypeptides harboring antigenic determinants recognized by the recipient organism or polypeptides associated with the synthesis of molecules comprising an antigenic determinant recognized by the recipient organism disrupted. In some embodiments, all or substantially all of the genes encoding polypeptides harboring antigenic determinants recognized by the recipient organism or polypeptides associated with the synthesis of molecules comprising an antigenic determinant recognized by the recipient organism are disrupted. As used herein “substantially all of the genes encoding polypeptides harboring antigenic determinants recognized by the recipient organism or polypeptides associated with the synthesis of molecules comprising an antigenic determinant recognized by the recipient organism” means at least 90% of the genes encoding polypeptides harboring antigenic determinants recognized by the recipient organism or polypeptides associated with the synthesis of molecules comprising an antigenic determinant recognized by the recipient organism. In other embodiments, at least 85%, at least 80%, at least 70%, at least 60%, at least 50%, at least 40%, at least 30%, at least 20%, or at least 10% of the genes encoding polypeptides harboring antigenic determinants recognized by the recipient organism or polypeptides associated with the synthesis of molecules comprising an antigenic determinant recognized by the recipient organism may be disrupted.

Genes which encode polypeptides harboring an antigenic determinant recognized by the recipient organism or polypeptides associated with the synthesis of molecules comprising an antigenic determinant recognized by the recipient organism may be identified using a number of techniques familiar to those skilled in the art. For example, in one embodiment, cDNA libraries are prepared from mRNA from the donor organism, or from a particular cell type, organ or tissue from the donor organism which is to be used in xenotransplantation. A variety of techniques are available for preparing cDNAs.

The resulting cDNAs are inserted into an expression vector such that they are operably linked to a promoter. Preferably, the expression vector also encodes a marker which allows cells containing the expression vector to be distinguished from cells which do not contain the expression vector. For example, the marker may be a selectable marker which allows cells containing the vector to replicate in the presence of a drug. Alternatively, the marker may be a polypeptide which is easily detected, such as green fluorescent protein, red fluorescent protein, CD8, flag tag, HA tag, C-myc, GST, mbp, polyhistidine, and the like.

The expression vectors are introduced into host cells in which the promoter is functional such that the polypeptides encoded by the cDNAs are produced in the host cells. The host cells may be any type of cell capable of expressing the polypeptides encoded by the cDNAs from the promoter. For example, the host cells may be bacterial cells, yeast cells, insect cells, or mammalian cells. In some embodiments, the host cells may be HeLa cells, HEK 293T cells, and the like, for example.

Preferably, in order to facilitate identification of genes which encode polypeptides from the donor organism which harbor an antigenic determinant, only a single cDNA is introduced into each of the host cells. This may be achieved, for example, by infecting the host cells with retroviruses encoding the polypeptide at a level of multiplicity such that each cell is only infected by a single virus.

The cells expressing the polypeptides encoded by the cDNAs from the donor organism are contacted with naturally occurring immunoglobulin family proteins. “Naturally occurring immunoglobulin family proteins” is meant to be defined broadly as polypeptides that contain an immunoglobulin domain, and occur naturally in the proposed recipient organism. These proteins upon contact with polypeptides, lipids, carbohydrates, and any other molecule comprising an antigenic determinant, as well as combinations thereof, are capable of signaling the presence of an antigenic determinant which is recognized to a recipient organism. One who is skilled in the art will appreciate that naturally occurring immunoglobulin family proteins include many different types of molecules and, are present on the surface of many different cells. For example, without limitation the naturally occurring immunoglobulin family proteins may be present in one or a combination of any of the following: sera from one or more recipient organisms, such as human beings; a polyclonal antibody population or an enriched polyclonal antibody population from one or more recipient organism; any other immunoglobulin(s) from one or more recipient organism; or be present on the surface B-cells, T-cells, including CD4+ and/or CD8+ cells, dendritic cells, macrophages and natural killer cells (NK cells) from one or more recipient organism; and any other suitable cell or molecule from one or more recipient organism. Thus, the term “naturally occurring immunoglobulin family proteins” includes antibodies, B-cell receptors, T-cell receptors, MHC molecules, cellular receptors, cell surface molecules, and the like.

In some embodiments, polypeptides encoded by the cDNAs from the donor organism are contacted with naturally occurring immunoglobulin family proteins, which can include, for example, immune sera from the recipient organism under conditions which permit any antibodies in the sera which recognize the polypeptides to specifically bind to their target antigenic determinants. After washing off non-specifically bound antibodies, cells expressing cDNAs encoding polypeptides from the donor organism which are specifically bound by the antibodies from the recipient organism are identified. The cells encoding such polypeptides may be identified by a variety of methods familiar to those skilled in the art. For example, a detectably labeled secondary antibody directed against antibodies from the recipient organism may be used to detect the binding of antibodies from the recipient organism to a polypeptide from the donor organism. In some embodiments, the secondary antibodies may be labeled with a fluorescent moiety and fluorescence activated cell sorting (FACS) may be used to obtain the cells expressing the polypeptides recognized by the recipient organism. In some embodiments, magnetic beads may be used to select the cells expressing the antigenic determinants recognized by the immunoglobulin derived from the sera of the recipient organism.

An other approach to identify antigenic determinants is to use either cells from pig tissue or human cells such as HeLa cells to HEK 293T cells expressing the cDNA library in the pCDNA3 vector. These cells can be subjected to subcellular fractionation to purify the cell surface fraction. The proteins in the cell surface fraction are subject to two-dimensional gel electrophoresis followed by SDS-PAGE. This gel is then transferred to nitrocellulose and probed with the naturally occurring Immunoglobulin family proteins derived from the recipient, which may or may not have been pre absorbent on a column removing the antibodies directed against Gal-3. This approach may also be used to identify any potential intracellular antigenic determinants, then whole cells lysates is used instead of cell surface fractions.

The genes encoding the polypeptides harboring an antigenic determinant recognized by the recipient organism are sequenced using standard technology. To prevent or reduce recognition of the identified polypeptides by the recipient organism, a desired number of the genes encoding the polypeptides are disrupted in cells from the donor organism. The genes may be disrupted in any cell from the donor organism which is capable of being used to generate a cell, organ or tissue to be used in a xenotransplantation procedure or other medical procedure. For example, the genes may be disrupted in primary skin fibroblasts, granulosa cells, and primary fetal fibroblasts, stem cells, germ cells, fibroblasts or non-transformed cells from any desired organ or tissue.

The genes may be disrupted using a variety of technologies familiar to those skilled in the art. For example, a stop codon may be introduced into the gene by homologous recombination. Alternatively, a deletion may be introduced into the gene by homologous recombination. In some embodiments, stop codons may be introduced in all reading frames in the sequence downstream of the deletion to eliminate artifactual translation products. In further embodiments, the gene may be disrupted by inserting a gene encoding a marker protein, for example, therein via homologous recombination. It will be appreciated that the deletion, stop codon, marker gene, or other disruption may be located at any position which prevents or reduces recognition of the antigenic determinants by the immune system of the recipient organism. Thus, the stop codon, deletion, marker gene, or other disruption may prevent expression of polypeptides harboring the antigenic determinants on the surface of the donor cells by preventing expression of a fully functional polypeptide in the donor cells, interfering with the transport of the polypeptide to the cell surface, interfering with the folding of the polypeptide or any other mechanism which prevents or reduces recognition of the antigenic determinants by the immune system of the recipient organism. Preferably, if the donor cells are diploid, both chromosomal copies of the gene are disrupted in the donor cells.

Genes encoding polypeptides harboring antigenic determinants recognized by the recipient organism may be sequentially disrupted in cells from the donor organism to generate cells in which each of the desired genes have been disrupted. If desired, after disruption of each of the genes in the cells from the donor organism, the cells may be contacted with serum from the recipient organism to confirm that recognition of the polypeptides encoded by the genes by the recipient organism has been reduced or eliminated. Alternatively, if desired, more than one gene encoding a polypeptide harboring an antigenic determinant recognized by the recipient organism may be disrupted in a single step. The disruption procedure is repeated until cells from the donor organism having the desired number of genes disrupted have been generated.

In some embodiments of the present invention, the donor cells having the desired genes disrupted are then used to generate organs or tissues which have the desired genes disrupted. A variety of techniques may be used to generate the organs or tissues. For example, in one embodiment, the donor cells are used to generate a genetically modified organism, such as a knockout animal, for example, comprising tissues or organs in which the desired genes have been disrupted. A variety of techniques for generating transgenic or genetically modified animals are familiar to those skilled in the art. For example, in some embodiments, the nuclei of the donor cells are removed and transferred into enucleated oocytes capable of developing into a transgenic or genetically modified animal. The oocytes may be from the same species as the donor cells or from a different species. The oocytes comprising the nuclei from the donor cells are then introduced into an organism in which they can develop into a transgenic or genetically modified animal. The oocytes may be introduced into an organism from the same species as the donor cells and/or the oocytes or from a different species from the donor cells and/or the oocytes. The oocytes are allowed to develop into genetically modified organisms and, after birth, the transgenic or genetically modified organisms are allowed to grow until their tissues or organs are suitable for use in a xenotransplantation procedure. The genetically modified animal may also be generated by co-injection of the components necessary to induce the homologous recombination together with sperm into the oocyte. (Perry, A. C. F. et al. Nat Biotechnol. November 2001 ; 19(11):1071-3 Efficient metaphase II transgenesis with different transgene archetypes, the disclosure of which is incorporated herein by reference in its entirety.). These methods are discussed in more detail herein.

Alternatively, the donor cells having the desired genes disrupted may be seeded on an artificial scaffold which forms the support for the tissue or organ. The scaffold may be a synthetic polymer or may have a biological component, such as a collagen. Such matrices have been described in U.S. Pat. No. 6,051,071, the disclosure of which is incorporated herein by reference in its entirety. Donor cells having the desired genes disrupted are grown on the scaffold. The scaffold comprising the donor cells is then implanted into the recipient organism.

In other embodiments, cells which are not associated with one another to form a tissue or organ and which have the desired genes disrupted may be administered directly to a recipient in need of a beneficial factor provided by the cells. For example, in one embodiment, the donor cells may be brain cells or fetal brain cells which produce dopamine. In this embodiment, the brain cells or fetal brain cells are introduced into an individual suffering from Parkinson's disease. In other embodiments brain or fetal cells are introduced into individuals suffering from Alzheimer's disease. In another embodiment, the donor cells may be stem cells which have been allowed to differentiate into a desired cell type. For example, the stem cells may be allowed to differentiate into muscle cells, such as heart muscle cells, bone cells, islet cells, skin cells, nerve cells, or endothelial cells. The cells are introduced into a recipient in need thereof. For example, the recipient may be suffering from a spinal cord injury, stroke, bums, heart disease, osteoarthritis or rheumatoid arthritis, or diabetes. In a particular embodiment of the present invention, donor heart muscle cells prepared in accordance with the present invention may be transplanted into a recipient suffering from heart disease. In another embodiment of the present invention, donor islet cells prepared in accordance with the present invention may be introduced into a recipient suffering from diabetes.

In another embodiment, one or more genes encoding an MHC protein endogenous to the donor cell, is replaced with one or more genes encoding an MHC protein from the desired recipient organism.

In another embodiment of the present invention, donor cells having the desired genes disrupted are genetically engineered to express a polypeptide beneficial to the recipient. For example, the donor cells may be genetically engineered to express a growth factor or cytokine. In one embodiment, the donor cells may be genetically engineered to express a polypeptide whose absence or production at insufficient levels has caused a disease in the recipient organism. In another embodiment, the vector may encode a factor which inhibits the activity or reduces the amount of a nucleic acid, polypeptide, carbohydrate, lipid or any other molecules whose production at abnormally high levels has caused a disease in the recipient organism. In an other embodiment molecules are expressed by the transgenic or genetically modified animal that diminish rejection of the transplanted organ, tissue or cells.

The following examples are intended to illustrate some embodiments of the present invention. It will be appreciated that the following examples are exemplary only and that the scope of the present invention is defined by the appended claims. In particular, it will be appreciated that any methodologies familiar to those skilled in the art may be substituted for those specifically enumerated in the examples below. Further, it will be appreciated that although certain organisms or cells are used in the following examples other organisms or cells which are consistent with the intent of the present invention may be substituted.

### EXAMPLE 1

### Preparation of cDNA Libraries

### Example 1A

cDNA libraries are prepared from polyA+ RNA from the organism, cell type, tissue, or organ which is to serve as the donor in xenotransplantation. For example, if the donor organism is a pig, the mRNA may be prepared from any desired cells, tissue or organ, including but not limited to kidney, liver, pancreas, heart, heart valve, lung, intestine, brain, cornea, endothelial cells or peripheral blood cells. If desired, the cDNA libraries may be obtained from a commercial source such as Clontech (Palo Alto, Calif.) after supplying the source with tissue, total RNA or polyA+ mRNA.

Alternatively, cDNA libraries are prepared using polyA+ RNA isolated from donor organs obtained from a local slaughterhouse. In preferred embodiments, the mRNAs were obtained from one or more of the following organs: kidney, liver, pancreas, brain, heart, heart valve, cornea, lung, intestine and endothelial cells from the big vessels.

An RNA preparation kit is obtained from Invitrogen (Carlsbad, Calif.) and mRNA is prepared according to the manufacturer's instructions. Briefly, the selected organs are individually homogenized and the cells are lysed in RNAse free lysis buffer. The lysate is passed through an 18-21 gauge needle. PolyA+ RNA is isolated by incubating the lysate with oligo(dt) cellulose in batch and rotating. The oligo(dt) cellulose is transferred to a column and extensively washed before the RNA is eluted off the oligo(dt) cellulose. The quality and the quantity of the mRNA are monitored by visualization of the mRNA by agarose gel electrophoresis and by optical density (OD) respectively.

The mRNA obtained as described above is then used to prepare double stranded cDNA using a modification of the protocol described in Huynh et al., 1984, DNA Cloning 1:49-78, the disclosure of which is incorporated herein by reference in its entirety. Briefly, mRNA is converted into double-stranded DNA having unique ends which facilitate directional cloning into a vector, such as a retrovirus vector. First, the mRNA is hybridized to a linker-primer that incorporates a poly(dt) tract (at its 3′ end) as well as a restriction site for Not I. The linker-primer is extended using an RNAse H⁻ version of the Moloney murine leukemia virus transcriptase (Super Script, Gibco, BRL) and a nucleotide mix in which dCTP is replaced with 5-methyl-dCTP. When first strand synthesis is completed, the reaction mixture is transferred into a second tube that contains the pre-chilled second-strand mixture. The second strand is synthesized using RNAse H and E. Coli DNA polymerase I. Finally, a blunting step (consisting of treatment with mung bean nuclease and Klenow fragment is carried out to prepare the cDNA for ligation to a linker, such as an EcoRI linker.

It will be appreciated that cDNA may also be prepared using any other methodology familiar to those skilled in the art including that set forth below in Example 1B.

### Example 1B

A 2 month old male Yucatan mini-pig was purchased from a local slaughterhouse. The pig was sacrificed and the kidneys, heart, liver, lung, heart valve, intestine and other tissues were immediately collected. The tissues were diced and placed into 50 ml conical tubes, snap frozen in an ethanol-dry ice bath and stored at −80° C. until needed.

The total RNA was extracted from the desired porcine tissue, in this example kidney, using RNA-Bee (Tel-Test Inc) as directed by the manufacturer. Alternatively, other methods of total RNA extraction can also be used. PolyA+ RNA was purified over an affinity column consisting of an oligo(dT) cellulose matrix (Roche) as directed by the manufacturer. The quality of the polyA+ RNA was monitored by visualization of the RNA by agarose gel electrophoresis and by Northern blot analysis with an α-[³²P]dCTP labeled GAPDH specific cDNA probe followed by autoradiography. The quantity of poly A+ RNA was monitored by optical density. The polyA+ RNA from kidney tissue obtained as described above was then used to prepare double stranded cDNA using the SuperScript™ Plasmid System with GATEWAY™ Technology for cDNA Synthesis and Cloning kit (Invitrogen). Briefly, porcine kidney polyA+ RNA was converted into double-stranded cDNA having unique ends which facilitate directional cloning into a vector. The vector can be a retroviral vector or a mammalian expression vector, such as pRETROstell or pcDNA3.1 respectively. It will be appreciated that cDNA may also be prepared using any other methodologies familiar to those skilled in the art including those set forth in below

The SuperScript™ Plasmid System with GATEWAY™ Technology for cDNA Synthesis and Cloning kit (Invitrogen) was used to synthesize double stranded cDNA starting from porcine kidney polyA+ RNA exactly as directed by the manufacturer for insertion into the pcDNA3.1 vector. For cDNA subcloned into pRETROstell, synthesis of the double stranded cDNA from porcine kidney polyA+ RNA was carried out essentially as described by the manufacturer however a primer-adapter with an internal EcoRI restriction site and a linker with the Bam HI site were used to obtain cDNA directionality. The sequence of the EcoR1 primer adapter was 5′-p TCGAGAATTCT₁₂N₂[GAC]-3′ (SEQ ID NO: 10). The sequence of the BamH1 linker was

| | |
|---|---|
| 5′-GATCCGAAGGGGTTCG-3′ | (SEQ ID NO: 11) |
| 3′-GCTTCCCCAAGCp-5′ | (SEQ ID NO: 12) |

Furthermore, dCTP was replaced by methyl-dCTP (Roche) in the dNTP mix to protect the EcoRI sites from EcoRI endonuclease activity, which is used to achieve cDNA directionality. First strand synthesis was monitored by the incorporation of α-[³²P]-dATP instead of α-[³²P]-dCTP.

### EXAMPLE 2

### Insertion of cDNA into a Vector

### Example 2A

Blunt-ended, double-stranded cDNA obtained using the procedure described above in Example 1A, or any other suitable procedure, is methylated by EcoRI methylase, ligated to EcoRI linkers, and digested with EcoRI. The methylation step protects EcoRI sites in the cDNA from EcoRI digestion. The products of the restriction digest are passed over a Sepharose CL-4B column to remove unligated linkers or adapters and other low-molecular-weight material (less than 350 bp) that would interfere with cloning. The double-stranded cDNA is concentrated by ethanol precipitation. The resulting cDNA contains an Eco RI site at its 5′ end and a Not I site at its 3′ end, rendering it suitable for directional cloning. It will be appreciated that other restriction sites may also be used to facilitate directional cloning of the cDNAs.

The cDNA libraries are subcloned into a vector such as a retrovirus vector or any other suitable vector. For example, the vector may be pBMN-Z (Nolan lab, Onishi et al., 1996, Exp. Hematol. 24(2):324-329) or pLIB (Clontech, Palo Alto, Calif.). Vector DNA digested with the restriction enzymes EcoRI and Not I and cDNAs digested with EcoRI and Not I are ligated together. The ligation products are introduced into bacteria and the bacteria are plated on medium containing an appropriate agent for selecting colonies which contain the vector. DNA is prepared from individual colonies by the “mini-prep” procedure and digested with EcoRI and Not I to characterize the insert.

### Example 2B

The resulting double stranded cDNA from Example 1B was passed over a Sephacryl S-500 HR column (Invitrogen) to remove low-molecular-weight material, such as unligated linkers or adapters that would interfere with cloning, as directed by the manufacturer. The effluent was collected and analyzed as directed in the instructions of the SuperScript™ Plasmid System with GATEWAY™ Technology for cDNA Synthesis and Cloning kit (Invitrogen). 1 μl of each drop in which the Cerenkov counts were greater than background was separated by electrophoresis on a 1% agarose gel in parallel with DNA markers. The cDNA was transferred onto a Zeta Probe nylon membrane (Bio-Rad) in 40 mM NaOH by capillary action overnight. The nylon membrane was exposed to a film and the cDNA was visualized by autoradiography.

The size range of the synthesized cDNA was determined by comparing its migration to that of the molecular weight markers. Small (about 0.5 kb-1 kb), intermediate (about 1 kb-1.5 kb) and large (about 1.5 kb and larger) cDNA fragment (thereafter referred to as “library pools”) were pooled together after visualization by autoradiography and the double-stranded cDNA was concentrated by ethanol precipitation. The resulting cDNA, when synthesized as directed by the manufacturer, contained a SalI site at its 5′ end and a Not I site at its 3′ end, rendering it suitable for directional cloning. cDNA, synthesized as directed by the manufacturer but using the EcoRI primer adapter and the BamHI linker to obtain directionality, contained a BamHI site at the 5′ end and a EcoRI site at the 3′ end. Other restriction sites may also be used to facilitate directional cloning of the cDNAs.

The vector, whether it be pcDNA3, pRETROstell or any another vector is prepared in such a way to minimize empty plasmids in the library (background). To minimize background, an insert of 2 to 3 kb is introduced into the plasmid between the restriction sites to be used to construct the libraries. In this case, an insert is introduced between the Xho1 and Not 1 sites of pcDNA3 and between the BamHI and EcoRI sites of pRETROstell.

The plasmid constructs were first digested with one enzyme that will be used to insert the cDNA and that flanks the insert. In this example the Xho 1 restriction endonuclease was used to digest pcDNA3.1 and the Bam H1 restriction endonuclease was used to digest pRETROstell, using 5 units of enzyme/μg of plasmid DNA. The resulting plasmid was electrophoresed on a gel containing an appropriate percentage of agarose and the digested plasmid is separated from any remaining undigested product. The single digested plasmid was cut out of the gel and is gel purified. The purified plasmid DNA is next digested with the second enzyme flanking the insert and at the site of cDNA insertion; Not1 for pcDNA3.1 and EcoR1 for pRETROstell using 5 units of enzyme/μg of DNA. The digest reaction was electrophoresed on a gel containing an appropriate percentage of agarose. The double digested plasmid will migrate differently from any remaining single digested plasmid and the released insert. The DNA corresponding to the double digested plasmid DNA and the DNA insert were cut out of the gel and recovered using a gel purification kit (Quigen). An aliquot of the digested plasmid and insert as well as a known quantity of lambda phage DNA digested with HindIII were electrophoresed side by side on an agarose gel. The concentration of the digested plasmid and insert was estimated in ng/μl by visualization of the plasmid or insert and directly compared to known quantities of marker DNA.

The ligation of the cDNA to the linearized recipient vector was optimized. Test ligations, in which the ratio of linearized plasmid DNA to cDNA insert were varied, were performed. T4 DNA ligase (Gibco) catalyzed the ligation reaction and was used according to the manufacturer's directions. The resulting reactions were ethanol precipitated and resuspended in an appropriate volume of water and about 1 ng of each ligation reaction was electroporated into DH10B Electromax bacteria (Invitrogen) as directed by the manufacturer. An aliquot of each reaction was plated onto LB-agar plates containing appropriate antibiotics for selecting colonies that contain the plasmid and grown overnight at 37° C. The number of colonies obtained from each ligation reaction was counted and the optimal ratio of plasmid to cDNA insert was determined. Mini-prep DNA was prepared from 50 individual colonies per library pool and digested with the appropriate restriction endonucleases to determine the average size of the cDNA inserts. It will be appreciated that the average insert size of a quality cDNA library is of at least 1 kb or greater.

The cDNAs were subcloned into a vector, pcDNA3.1 or pRETROstell, and the small, intermediate and large cDNAs were independently ligated to the vector using an optimal vector to insert ratio. cDNAs with the restriction sites Not1 and Sal1 at its termini were subcloned into the Xho1 and Not1 sites of pcDNA3.1. cDNAs with terminal EcoR1 and BamH1 sites were subcloned into the BamH1 and EcoRI sites of pRETROstell.

It will be appreciated that the cDNAs may be subcloned into vectors other than those specifically listed above and that any suitable vector may be used. In these experiments, a minimum of 3×10⁶ primary bacterial transformants per library pool are obtained.

### EXAMPLE 3

### Expression of Polypeptides Encoded by cDNAs

### Example 3A

When the cDNAs were subcloned into the appropriate vectors, larger amounts of DNA are prepared from bacteria containing the vector. If the cDNAs were cloned into retroviral vectors, a packaging cell line was transiently transfected with the vectors containing the cDNAs by the calcium-phosphate method in the presence of chloroquine to inhibit lysosomal DNAses. For example, Phoenix cells (Garry Nolan, Stanford University) or the AmphoPack™-293 Cell Line (Clontech, Palo Alto, Calif.) were used as packaging cell lines.

24 hours post-transfection, the packaging cell line was detoxified by removing the chloroquine and the target cells to be infected with the retroviruses were split and prepared for infection. Preferably, the target cells to be infected with the retrovirus were derived from the recipient organism such that endogenous polypeptides expressed by the target cells will not be recognized by serum from the recipient organism. For example, if the recipient organism was a human being, HeLa, NIH 3T3 cells (or other suitable human cell lines) were split and prepared for infection. The supernatant from the packaging cell line, which contains virus particles comprising the cDNAs, was used to infect the target cell line 48 hours post-transfection. Preferably, infection was performed at a multiplicity of infection such that each infected cell contained only a single retrovirus carrying a single cDNA. In this way, each infected cell expressed only one polypeptide from the donor organism, while the population of infected cells expressed all or most of the proteins expressed in the tissue or organ from which the cDNAs were generated.

Preferably, the target cells to be infected with the retrovirus were derived from the recipient organism such that endogenous polypeptides expressed by the target cells will not be recognized by serum from the recipient organism. For example, if the recipient organism was a human being, HeLa, NIH 3T3 cells (or other suitable human cell lines) were split and prepared for infection.

It will be appreciated that other methodologies, vectors, and cell lines familiar to those skilled in the art may also be used to obtain a population of cells expressing all or most of the polypeptides expressed in the tissue or organ from which the cDNAs were generated. For example, the polypeptides may be expressed in bacterial cells, yeast cells, insect cells, or mammalian cells.

### Example 3B

As another alternative to the methods described in Example 3A, the following may be performed. Once the cDNAs have been subcloned into the appropriate vector (pcDNA3.1 or pRETROstell, for example as described herein) and a minimum of 3×10⁶ primary transformants per library pool was obtained, the plasmid cDNA library was expanded in semi-solid agar containing appropriate antibiotics at 30° C. for 40 hours. Bacteria containing plasmids with small, intermediate and large cDNA inserts were individually expanded and harvested. Plasmid DNA was then extracted from a fraction of the bacteria using the Qiagen Endotoxin-free plasmid extraction kit (Qiagen) as directed by the manufacturer. The remaining bacteria were frozen at −80° C. as glycerol stocks.

To express polypeptides endoded by cDNA sequences cloned into pRETROstell, Phoenix amphotropic cells or other appropriate packaging cell lines were transiently transfected with empty vectors (as a control) and vectors containing porcine cDNA inserts using Fugene 6 (Roche) using methods as recommended by the manufacturer. At some time post-transfection, usually but not exclusively at 48 and 72 hours, the supernatant containing the retrovirus produced by the packaging cell line was collected for infection of HeLa cells as described at the hypertext transfer protocol on the world wide web “Stanford.edu/group/nolan.” Preferably, the target cells to be infected with the retrovirus are derived from the recipient organism such that endogenous polypeptides expressed by the target cells will not bind xenoreactive antibodies present in the serum or immunoglobulins from the recipient organism. For example, if the recipient organism is a human being, HeLa, NIH 3T3, HEK 293T cells (or other suitable human cell lines) were prepared for infection. Cells expressing polypeptides comprising antigenic determinants recognized by the desired recipient organism were identified as follows below.

It will be appreciated that other methodologies, vectors, and cell lines familiar to those skilled in the art may also be used to obtain a population of cells expressing all or most of the polypeptides expressed in the tissue or organ from which the cDNAs were generated. For example, the polypeptides may be expressed in bacterial cells, yeast cells, insect cells, mammalian cells, and the like.

### EXAMPLE 4

### Identification of Cells Expressing Polypeptides Comprising an Antigenic Determinant Recognized by the Recipient Organism

### Example 4A

24-48 hours post-infection the HeLa cells or other cells expressing the proteins encoded by the cDNAs are ready for screening. In the case of HeLa cells, naturally occurring immunoglobulin family proteins, which was described above, was used to screen the cells. In this example the “naturally occurring immunoglobulin family proteins” comprise and are referred to as either “immunoglobulins” or “immunoglobulin composition.” One of skill in the art will appreciate that the naturally occurring immunoglobulin family proteins can be any, such as those described herein, or any other like composition.

Immunoglobulins from the desired recipient organism, such as those available in human sera, were obtained and used to screen for antigenic determinants by FACS analysis. If the desired recipient organism is a human being, the immunoglobulins are preferably obtained from people of different HLA haplotypes and blood groups in order to obtain a pool of antibodies representative of the human population. Mock infected HeLa cells were used as a negative control. Cells expressing the cell surface protein CD8 were used as a positive control.

The HeLa cells expressing the donor polypeptides were contacted with the immunoglobulins under conditions which permit antibodies to specifically bind to their targets. Again, as noted any other suitable cells may be used. After a wash step was performed to remove non-specifically bound antibodies, the specifically bound antibodies were contacted with a fluorescently labeled secondary antibody which binds to human antibodies under conditions which permit the secondary antibodies to specifically bind their targets. A wash step was performed to remove non-specifically bound secondary antibodies and the cells were passed through a fluorescence activated cell sorter. Fluorescent cells were collected. It should be noted that in some embodiments the secondary antibody may be conjugated to Biotin, and thus, it may be visualized by adding fluorescently labeled streptavidin.

### Example 4B

Alternatively, the following was performed. In the following example, HeLa cells were used but it is understood that any other appropriate cell line can be used. HeLa cells expressing the proteins encoded by the cDNAs were screened with naturally occurring immunoglobulin family proteins at some time post-transfection, usually but not exclusively limited to, 48 or 72 hours post-infection. Again, in some embodiments, the naturally occurring immunoglobulin family proteins in the present example comprise and are referred to as “immunoglobulins” or “immunoglobulin composition.” One of skill in the art will appreciate that, as discussed above, the naturally occurring immunoglobulin family proteins may comprise T cell receptors, polyclonal antibody populations, etc. For example, in some embodiments immunoglobulins from the desired recipient were used to screen for antigenic determinants by FACS analysis or by using magnetic beads such as those manufactured by Dynal (CELLection™ BiotinBinder Kit) or Miltenyi Biotec (MACS beads) according to the supplier's instructions. If the desired recipient organism is a human being, the immunoglobulins are preferably obtained from people of different HLA haplotypes and blood groups in order to obtain a pool of antibodies representative of the human population. Mock infected HeLa cells with empty pRETROstell were used as a negative control.

Purified human immunoglobulins (IgG, IgM and IgA) were purchased from various suppliers such as, for example, Rockland and Jackson Immunochemicals. Prior to cell labeling, the specific signal of the immunoglobulin binding reaction was optimized. To decrease non-specific signals, immunoglobulins (IgG, IgM and IgA) that bind to HeLa cells were removed by absorption by incubating 100 μg aliquots of IgG, IgM or IgA with 10 million HeLa cells in 500 μl of PBS containing 1% FCS overnight at 4° C. with constant shaking. The immunoglobulins were separated from the HeLa cells by centrifugation and the antibody was collected. This absorbed immunoglobulin was then depleted of antibodies recognizing the gal-3 epitope by subjecting it to a column consisting of a Gal α1-3 Gal β1-4Glcβ-Sepharose FF (Synthesome Ltd) and a Gal α1-3Gal β1-sepharose FF (Bdi) (Synthesome Ltd) matrix. Column chromatography was performed according to standard protocols. The anti-gal-3 antibody depleted immunoglobulins were collected, concentrated to about 1 μg/μl using centricon tubes (Millipore) and stored at −20° C. The human anti-gal-3 antibodies were recovered from the chromatography column by glycine elution and the antibody was neutralization, dialyzed against PBS and concentrated as described above.

The reactivity of human IgG, IgM or IgA depleted of reactive antibodies to HeLa cells and Gal-3 was tested on HeLa cells and primary pig skin fibroblasts. The HeLa cells and the primary pig fibroblasts were contacted with varying amounts of human IgG, IgM or IgA, ranging from 2 μg to 10 μg of antibody per million cells, under conditions which permit antibodies to specifically bind to their targets. Non-specifically bound antibodies were washed off and the specifically bound antibodies were contacted with a secondary anti-human Ig conjugated to biotin, which binds to human Ig under conditions which permit the secondary antibodies to specifically bind their targets. A wash step was performed to remove non-specifically bound secondary antibodies and the cells were incubated with streptavidin conjugated to PE. The cells were passed through a fluorescence activated cell sorter (FACS) and a FACS profile of human IgG, IgM or IgA reactivity to HeLa and primary pig fibroblast cells was obtained. The optimal amount of antibody to be used for screening the libraries will have low reactivity to HeLa cells and a high reactivity to primary pig fibroblasts.

Alternatively, to increase the titer of xenoreactive antibodies, purified membrane proteins isolated from porcine kidney or primary skin fibroblasts were conjugated to a matrix using the AminoLink® Plus Immobilization Kit (Pierce Biotechnology) as directed by the manufacturer to create an affinity column. Human immunoglobulins were added to the column and chromatography was carried out according to standard protocols. The human antibodies bound to the affinity column were those that specifically recognize and react against porcine membrane proteins. These enriched xenoreactive antibodies were collected, concentrated, quantitated and stored at −20° C.

To screen the porcine kidney cDNA library, HeLa cells expressing the donor polypeptides following infection with the small, medium or large cDNA library pools or the empty pRETROstell vector were contacted with the optimized human immunoglobulins described above under conditions which permit antibodies to specifically bind to their targets. After a wash step was performed to remove non-specifically bound antibodies, the specifically bound antibodies were contacted with a secondary anti-human Ig conjugated to biotin which binds to human antibodies under conditions which permit the secondary antibodies to specifically bind their targets. A wash step was performed to remove non-specifically bound secondary antibodies and the cells were incubated with streptavidin bound to PE. pRETROstell contains an independent ribosomal entry site flanking the porcine cDNA insert and EGFP allowing for the bicistronic expression of EGFP and the porcine protein of interest. All transfected cells should express EGFP in addition to the porcine cDNA sequence. The labeled cells were passed through a fluorescence activated cell sorter and HeLa cells double positive for PE and EGFP were collected.

### Example 4C

After the first round of sorting, including sorting by the above described methodologies in Examples 4A and 4B, the cells identified as expressing a polypeptide recognized by immunoglobulins were expanded to a larger number of cells and the foregoing FACS analysis was repeated two or more times. Cells recognized by the immunoglobulins after the final round of FACS sorting were grown up as single cell clones in 96 well plates and individually screened to isolate clones expressing porcine proteins, or antigenic determinants that react with immunoglobulins. Sequencing of the cDNA insert of immunoreactive clones is described in Example 5, but may also be performed by any other method familiar to the skilled artisan.

If desired, following the identification of the major antigenic determinants, the libraries of cells expressing polypeptides from the donor organism may be rescreened in order to identify minor antigenic determinants that could potentially have been obscured by major antigenic determinants. Immunoglobulins from the recipient organism, such as human immunoglobulins, are subjected to immunoabsorbtion to remove the antibodies recognizing major antigenic determinants. To perform immunoabsorption, cells expressing the polypeptides which were identified as harboring antigenic determinants recognized by the recipient organism using the methods above are incubated with immunoglobulins from the recipient organism. In some embodiments, the cells expressing the polypeptides which were identified as expressing polypeptides harboring antigenic determinants recognized by the recipient organism are fixed prior to incubating them with immunoglobulins from the recipient organism. The antibodies which recognize the previously identified polypeptides bind to the cells expressing these polypeptides while antibodies against other polypeptides, such as polypeptides comprising minor antigenic determinants, will remain in the immunoglobulin composition. The remaining composition which contains antibodies against other polypeptides, such as polypeptides comprising minor antigenic determinants, is removed and incubated with yet another batch of cells expressing the polypeptides which have already been identified as comprising an antigenic determinant recognized by the recipient organism to further deplete the immunoglobulin composition of any antibodies against these polypeptides. The immunoglobulin composition is then free of any antibody recognizing the previously identified polypeptides and is then used to re-screen the library of cells expressing polypeptides from the desired donor tissue or organ.

Immunoabsorbtion can be performed using individual clones which express a single polypeptide previously identified as harboring an antigenic determinant recognized by the recipient organism. In such an approach the immunoglobulin composition is sequentially depleted of antibodies against each of the previously identified polypeptides. Alternatively, immunoabsorption can be done using a combination of multiple clones expressing different polypeptides previously identified as harboring an antigenic determinant recognized by the recipient organism so as to deplete the immunoglobulin composition of antibodies against multiple polypeptides simultaneously.

Alternatively, an antigenic determinant comprising a polypeptide can be conjugated to a matrix to create an affinity column. The human antibodies recognizing this antigenic determinant can be removed by passing the immunoglobulins and collecting the eluate as described above.

The immunoglobulin composition from the recipient organism which has been depleted of antibodies which recognize major antigenic determinants is then placed in contact with the cells expressing polypeptides from the donor organism and a FACS analysis is performed as described above to identify additional polypeptides which are recognized by the recipient organism.

It will be appreciated that other methodologies familiar to those skilled in the art may also be used to identify polypeptides recognized by the human immune system. For example, if the polypeptides from the donor organism are expressed in bacterial cells, yeast cells, insect cells, mammalian cells, and the like, these cells may be contacted with immunoglobulins from the recipient organism (or any other naturally occurring immunoglobulin family proteins) and the cells which bind antibodies from the immunoglobulin composition may be identified using standard techniques, such as detectably labeled secondary antibodies.

### Example 4D

### Expression of Polypeptides Encoded by cDNAs Expressed in pcDNA3.1 and Screening of the Library

To screen the porcine kidney cDNA library constructed into pcDNA3.1, a procedure consisting of multiple rounds of transient transfections and immunoselections was performed essentially as described in Current Protocols in Molecular Biology (Published by John Wiley & Sons). HEK 293T cells were transfected with the empty pcDNA3.1 vector (as a control), the small, intermediate or large library pool of cDNAs or with the pcDNA3.1-EGFP construct (to monitor the transfection efficiency) using the Fugene 6 method (Roche). 72 hours after transfection, the HEK 293T cells expressing the donor polypeptides or the control pcDNA3.1 vector were contacted with human IgG, IgM or IgA, in which HEK 293T reactive antibodies have been removed, or with the xeno-enriched antibody, under conditions which permit antibodies to specifically bind to their targets. After a wash step was performed to remove non-specifically bound antibodies, the specifically bound antibodies were contacted with a secondary anti-human Ig antibody conjugated FITC. A wash step was performed to remove non-specifically bound secondary antibodies and the cells were passed through a fluorescence activated cell sorter and were collected in bulk. Approximately 1.6% of sorted HEK 293T cells transfected with the intermediate sized library were FITC positive. The plasmid DNA was isolated from the sorted cells and DH10B Electromax E.coli (Invitrogen) were transformed with the plasmid DNA.

HEK 293T cells are tranfected with the plasmid DNA by the spheroplast fusion method as described in Current protocols in Molecular Biology (Published by John Wiley & Sons) or by any inefficient method of transfection and four or more rounds of transfection and immunoselection are performed. Following the second-to-last selection, plasmid DNA is prepared from individual bacterial colonies and the plasmid DNA is used to transfect HEK 293T cells. HEK 293T cells expressing antigenic determinants are then identified by FACS analysis and the plasmid DNA recovered and the cDNA insert sequenced.

Again, one of skill in the art will appreciate that these methods can be adapted to express and screen any other cDNA library. Further, the skilled artisan will appreciate that any other suitable methods may be employed to express and screen such libraries.

### Example 4E

### PCR Cloning GGTA1 Coding Region from pcDNA 3.1 cDNA Libraries

As a starting template for PCR the following solutions were made: 10 μg/μl pcDNA3.1 containing porcine cDNA of the small and medium size fractions of porcine cDNA. The primer sequences were designed using the computer program Omiga 2.0™ (Genetics Computer Group), with the published full GGTA1 cDNA as the template. The sequence of GGTA1 is available with the accession number L36152, the disclosure of which is incorporated herein by reference in its entirety. The criteria for designing the primers was that they would be optimal for PCR and lie outside of the coding portion of the GGTA1 but lie within the GGTA1 cDNA. The resultant primers had the following sequences:

| | | |
|---|---|---|
| Forward | 5′-CATGAGGAGAAAATAATGAAT-3′ | (SEQ ID |
| | | NO: 13) |
| Reverse | 5′-CTGCTGGCACAATTTAAAG-3′ | (SEQ ID |
| | | NO: 14) |

A PCR reaction was set up as follows:—2.5 μl Expand Long PCR Buffer #3 (Roche), 2 μl 10 mM dNTPs (Roche), 1 μl of pcDNA Porcine cDNA library, 1 μl of forward primer, 1 μl of reverse primer, 1 μl Expand Long PCR Polymerase (Roche), and 16.5 μl of DNAse and RNAse free water. The PCR program used to clone this gene was as follows:

1. 94° C. for 10 minutes

2. 94° C. for 45 seconds

3. 54° C. for 45 seconds

4. 68° C. for 2 minutes 30 seconds

5. Go to step 2, 10 times

6. 94° C. for 45 seconds

7. 54° C. for 45 seconds

8. 68° C. for 2 minutes and 30 seconds, increase by 5 seconds per cycle

9. Go to step 6, 20 times

10. 68° C. for 10 minutes

11. Hold at 10° C.

This PCR reaction produced a band of DNA that was 1.1 kB long, the expected size for GGTA1's coding region. This fragment of DNA was cloned into pGEM-T Easy (Promega) a vector designed to receive PCR fragments. Using sequence primer sites in pGEM-T the DNA fragment was sequenced using standard methods. A clone containing a perfect match to the published sequence was identified in this way.

### Example 4F

### Screening the pcDNA3.1 Porcine Kidney Library for gal-3 Expression

HEK 293T cells, plated 24 hours earlier, were transfected using the Fugene 6 reagent (Roche) at a ratio of 3:2 as described by the manufacturer. The transfected plasmid DNA consisted of either pcDNA3.1, the intermediate fraction of the porcine kidney cDNA library or a pcDNA3.1-EGFP construct. The transfection efficiency was determined by FACS analysis by measuring the proportion of EGFP positive cells at 72 hours after transfection with pcDNA3.1-EGFP. The HEK 293T cells transfected with empty vector or the intermediate sized library were incubated with a purified primary baboon anti-gal-3 IgG (Chemicon International). 1 μl of antibody was used to label one million cells for 1 hour at 4° C. The primary antibody was washed off and cells were incubated with the secondary antibody consisting of goat anti-monkey IgG conjugated to FITC in addition to 7-AAD for 30 minutes at 4° C. using standard quantities. The cells were washed and immediately subjected to FACS analysis to determine the proportion of live cells labeled with FITC. The FACS profile of the HEK 293T cells transfected with empty vector was considered as background. The FACS profile of the HEK 293T cells transfected with the intermediate fraction of the porcine kidney cDNA library showed a significant shift compared to the control transfected HEK 293T cells. About 20% of live cells were FITC positive indicating that gal-3 is produced on the surface of library transfected cells and is detected by the gal-3 antibody. This result was reproducible.

Expression of this porcine GGTA1 in human 293T cells was demonstrated to be sufficient to cause the expression of the Gal-3 carbohydrate in these cells, detected with a commercial antibody against Gal-3. Subsequently, 293T cells expressing GGTA1 were used as a positive control to optimize preparations of immunoglobulins used to screen cDNA libraries for novel antigenic determinants.

### Example 4G

### PCR Cloning Genes from the pRETROstell Vector

Once an infected cell has been identified as expressing a polypeptide comprising an antigenic determinant which is recognized by the desired recipient organism, the inserted gene encoding that polypeptide is cloned in the following way. The single cell is allowed to divide in culture. Once the cellular population has reached a certain size, genomic DNA is prepared from these cells. Genomic DNA is prepared using the High Pure PCR Template Preparation Kit (Roche) following manufacturer's instructions. The manufacturer recommends using between 10 000 and 100 000 000 cells.

Once the genomic template DNA has been prepared, the inserted gene is amplified using primers specific for pRETROstell. Their sequences are:

| | | |
|---|---|---|
| Forward | 5′-AAAGTAGACGGCATCGCAGC-3′ | (SEQ ID NO: 15) |
| and | | |
| Reverse | 5′-CACACCGGCCTTATTCCAAGC-3 | (SEQ ID NO: 16) |

The PCR is performed using the Expand Long Template PCR system (Roche). The PCR reaction is carried out with an annealing temperature of 58° C., an elongation temperature of 68° C. and 35 amplification cycles. Progressively longer elongation steps are used to increase the chances of amplifying longer DNA inserts. According to the manufacturer this system can be used to clone fragments up to 15 kiloBases in length. The PCR program that is used is initially optimized to clone fragments up to 3 kiloBases in length, which should cover the majority of genes, and then optimized to clone longer fragments if this fails. The Expand Long Template kit contains a combination of high fidelity enzymes, so it is possible to clone a perfect copy of the inserted gene.

Following amplification of a desired gene, the PCR product is run on an agarose gel and then gel purified using QIAquick spin columns (Qiagen). The purified fragments of DNA are cloned into pGEM-T Easy (Promega), a-vector that facilitates amplification and sequencing of PCR products. The ends of the gene identified in this way are sequenced using an ABI automated sequencer. The resultant information is used in a BLAST program search to check for homology with previously identified genes in other species. In addition to this, the sequence is used to screen the cDNA library to identify a perfect, full length cDNA for the gene. Either the PCR or the cDNA identified sequences are cloned back into pRETROstell. This vector is then used to re-infect HeLa cells, to produce a population of cells expressing only the identified gene. These cells are used to re-confirm that the gene identified is the one that is causing the reactivity to antibodies from the recipient organism.

### EXAMPLE 5

### Sequencing Antigenic Determinants Identified using pcDNA3.1

One a given pcDNA3.1 has been identified as containing a cDNA that encodes an antigenic determinant or that leads to the production of an antigenic determinant, that plasmid DNA is isolated from bacteria using the Wizard Plus SV Miniprep DNA Purification System (Promega) following manufacturer's instructions. This DNA is the sequenced using an ABI automated sequencer using the following primers.

| | |
|---|---|
| For the 5′ end of the gene | |
| 5′-TAATACGACTCACTATAGGG-3′ | (SEQ ID NO: 17) |
| For the 3′ end of the gene | |
| 5′-AATGCGATGCAATTTCCTC-3′ | (SEQ ID NO: 18) |

One of skill in the art can easily apply the above methods with appropriate primers to sequence any cloned DNA. Genes encoding the cDNA inserts can be sequenced according to any method familiar to the skilled artisan.

### EXAMPLE 6

### Preparation of Antibodies which Recognize the Polypeptides Encoded by cDNAs

After each cDNA from the donor organism which encodes a polypeptide comprising an antigenic determinant recognized by naturally occurring immunoglobulin family proteins, such as for example sera from the recipient organism, is identified, affinity purified antibody which specifically binds to the encoded polypeptide is prepared as follows. The clones containing cDNAs encoding polypeptides comprising antigenic determinants recognized by serum from the recipient organism are expanded and sera from the recipient organism is immunoabsorbed onto the cells. The cells are washed with a washing buffer to remove nonspecifically bound antibody. The antibodies specifically bound to the corresponding cell population are then eluted off the cells to provide an affinity purified antibody which binds to the antigenic determinant in the polypeptide from the donor organism.

The affinity purified antibodies prepared as described above may be used to check for expression of the antigenic determinants encoded by the cDNAs in cDNA libraries, to check for the presence of the antigenic determinants on the cell types which are to be used to generate cells which do not express these antigenic determinants as described below, to check for the presence of the antigenic determinants on cells in which the genes encoding the polypeptides comprising the antigenic determinants have been disrupted, and to check for the presence of the antigenic determinants on different organs from the donor organism in order to map the tissue distribution of the antigenic determinants in the donor organism.

Alternatively, if the antigentic determinant identified is a polypeptide for which the cDNA has been identified, then a pure recombinant protein for that cDNA can be produced. The protein is expressed and purified. Then, it is attached to a sepharose column and it is used to purify antibodies. One of skill in the art will appreciate that any suitable method may be used to prepare antibodies that recognize the polypeptides.

### EXAMPLE 7

### Further Analysis of Antigenic Determinants

If desired a further analysis of antigenic determinants to complement the FACS analyses described above may be performed as follows. The HeLa cells, HEK 293T cells or other suitable cells, expressing the polypeptides encoded by the cDNA library from the donor organism are subjected to subcellular fractionation, as described by Liljedahl et al., 1996, EMBO J. 15(18):4817-4824, the disclosure of which is incorporated herein by reference in its entirety. In another embodiment cells from pig tissue will be subjected to subcellular fractionation. Briefly, plasma membrane fractions are collected and subjected to two dimensional gel electrophoresis and then Western blotting. The blots are contacted with sera from the recipient organism to confirm that the antigenic determinants identified by FACS analysis are recognized by sera from the recipient organism, and new antigenic determinants can also be identified if the sera is preabsorbed with a clone expressing Galα1-3Galβ1-4GlcNAc-R (αGal) prior to using it to probe. Naturally occurring immunoglobulin family proteins other than one comprising sera from the recipient organism can also be used.

If desired, the blots may be probed with sera from the recipient organism which has been preabsorbed with all clones identified as expressing a polypeptide harboring an antigenic determinant using the FACS analyses described above in order to facilitate the identification of any additional polypeptides harboring an antigenic determinant recognized by the recipient organism which were not detected in the FACS analyses. In addition, if desired, the blots may be probed with sera which has been preabsorbed with polypeptides or other biomolecules comprising any known major antigenic determinants. For example, if the donor organism is a pig and the recipient organism is a human, the sera maybe be preabsorbed with Galα1-3Galβ1-4GlcNAc-R (αGal), a major porcine antigenic determinant recognized by humans, to remove the background of glycosylated proteins and facilitate the identification of additional antigenic determinants recognized by the recipient organism. The blots may also be probed with serum collected from patents exposed to pig tissue.

It will be appreciated that other methodologies familiar to those skilled in the art may also be employed to further analyze the polypeptides encoded by the cDNAs and the products of these antigenic determinants to identify those harboring antigenic determinants.

### EXAMPLE 8

### Identification of Intracellular Proteins Recognized by the Recipient Organism

If desired, intracellular proteins which harbor antigenic determinants recognized by the recipient organism may be identified as follows. Reduction or elimination of the presence of such antigenic determinants on a donor organ or tissue may avoid or reduce the possibility that during a later stage of transplantation leakage of intracellular proteins will occur and a T-cell response will be initiated against those antigens. Thus, if desired, intracellular proteins harboring antigenic determinants may be identified as follows. Whole cell lysates of the organs or tissues to be used as donors are prepared and subjected to two dimensional gel electrophoresis and Western blotting as described above. The blots are probed with sera from the recipient organism, or any other naturally occurring immunoglobulin family proteins, which have been preabsorbed with all cell clones previously identified as containing a cDNA encoding a polypeptide harboring an antigenic determinant recognized by the recipient organism. If desired, the sera may be preabsorbed with other known major antigenic determinants recognized by the recipient organism. For example, if the donor organism is a pig and the recipient organism is a human, the sera may be preabsorbed with a clone expressing Galα1-3Galβ1-4GlcNAc-R (αGal) prior to using it to probe the blot. The blots may also be probed with serum collected from patents exposed to pig tissue.

### EXAMPLE 9

### Generation of Cells in Which the Genes Encoding Polypeptides Which Harbor an Antigenic Determinant have been Disrupted

Genes encoding polypeptides which harbor an antigenic determinant recognized by the recipient organism which have been identified using the methods above are disrupted in cells suitable for use in obtaining donor organs or tissues. For example, the genes may be disrupted in cells suitable for use in nuclear transfer procedures, stem cell or germ cell-based procedures, or techniques in which tissues or organs are grown on a scaffold. Cells suitable for use in nuclear transfer procedures include but are not limited to one or more of the following cells: primary skin fibroblasts, granulosa cells, and primary fetal fibroblasts, fibroblasts or non-transformed cells from any desired organ or tissue.

In some embodiments, the genes are disrupted in pig cells. Primary pig fibroblasts may be obtained from skin incisions in adult pigs. A piece of tissue is removed and placed in tissue-culture media to obtain primary cell lines (Kubota et al., 2000, Proc. Natl. Acad. Sci. U.S.A. 97(3):990-995, the disclosure of which is incorporated herein by reference in its entirety).

Pig granulosa cells may be obtained as follows. Follicular fluid is aspirated from follicles of super ovulated 7-8 month old pigs 28-51 hours after induction of ovulation. (Polejaeva et al., 2000, Nature 407(6800):86-90, the disclosure of which is incorporated herein by reference in its entirety).

Primary pig fetal fibroblasts may be prepared from porcine cells from 35-day old fetuses (Schnieke et al., 1997, Science 278(5346):2130-2133, the disclosure of which is incorporated herein by reference in its entirety).

Cells in which one or more genes encoding a polypeptide harboring an antigenic determinant recognized by the recipient organism have been disrupted may be generated as follows. In some embodiments, cells in which a plurality of genes encoding polypeptides harboring an antigenic determinant recognized by the recipient organism have been disrupted are generated. The cells may have any desired number of genes encoding a polypeptide harboring an antigenic determinant recognized by the recipient organism disrupted. For example, the cells may have at least two, at least 4, at least 5, at least 10, at least 15, at least 20, at least 25, at least 35, at least 40 or more than 40 genes encoding polypeptides harboring antigenic determinants recognized by the recipient organism disrupted. In some embodiments, all or substantially all of the genes encoding polypeptides harboring antigenic determinants recognized by the recipient organism are disrupted. As used herein “substantially all of the genes encoding polypeptides harboring antigenic determinants recognized by the recipient organism” means at least 90% of the genes encoding polypeptides harboring antigenic determinants recognized by the recipient organism. In other embodiments, at least 85%, at least 80%, at least 70%, at least 60%, at least 50%, at least 40%, at least 30%, at least 20%, or at least 10% of the genes encoding polypeptides harboring antigenic determinants recognized by the recipient organism may be disrupted.

If more than one gene encoding an antigenic determinant recognized by the recipient organism is disrupted, the genes may be sequentially disrupted one at a time using any of a variety of methods familiar to those skilled in the art. In addition to the genes identified as encoding a polypeptide harboring an antigenic determinant using the methods described above, any genes previously known to encode a polypeptide harboring an antigenic determinant are also disrupted. For example, if the cells in which the disruptions are constructed are pig cells and the recipient organism is a human, the gene responsible for production of the known antigenic determinant gal-3 is disrupted. The genes may be disrupted in any desired order.

Techniques which may be used to disrupt genes encoding polypeptides harboring an antigenic determinant include, but are not limited to the following. In one method, the homologous recombination method described in Capecchi, 1989, Science 244(4910):1288-1292, the disclosure of which is incorporated herein by reference in its entirety, is used to generate disruptions. In this method, homologous recombination constructs comprising the genomic region containing coding sequence or a portion of the coding sequence of the gene encoding a polypeptide harboring an antigenic determinant in which an in frame stop codon has been introduced near the 5′ end of the coding sequence or comprising the coding region that has been replaced by a marker gene are introduced into the cell using methods such as lipofection, calcium phosphate transfection, electroporation or other methods familiar to those skilled in the art.

For example, to identify the genomic DNA with which to begin making such a construct, a genomic library from the donor organism, a tissue or organ from the donor organism, or from the cells in which the genes are to be disrupted is obtained. Nucleic acids comprising the coding sequence of the gene to be disrupted out or a portion thereof are obtained from the genomic library by excising the gene or portion thereof from the library using restriction enzymes or by generating an amplicon comprising the gene or portion thereof by PCR. For example, to obtain probes suitable for use in identifying genes encoding polypeptides comprising antigenic determinants recognized by the recipient organism in the genomic library, PCR primers for a given coding region (either the GGTA1 coding region or a coding region encoding a polypeptide comprising an antigenic determinant recognized by the recipient organism identified using the cDNAs obtained as described herein) that span the length of the coding region in a series of 200 base pair steps. Primers may be designed using any of the methods familiar to those skilled in the art, including the Omiga 2.0™ (Genetics Computer Group). PCR reactions are conducted using all of these primer pairs on genomic DNA isolated from the donor organism. For example, if the donor organism is pig, the genomic DNA may be from a Yucatan pig or another variety of pig. If a primer pair produces a amplification product from genomic DNA which is the same size as the distance between the sequences corresponding to the primers in the cDNA encoding the polypeptide comprising an antigenic determinant recognized by the recipient organism, then both of the primers lie in the same exon.

Pairs of primers lying in the same exon identified as described above are then used to screen a genomic BAC (Bacterial Artificial Chromosome) library from the donor organism for the genomic region containing the gene to be disrupted. For example, if the donor organism is a pig, the PCR screening may be performed using the pig genomic BAC library which is commercially available from the Human Genome Mapping Project (HGMP) Resource Centre in Cambridge UK. The HGMP Resource Centre provides the pig genomic BAC library as a series of progressively less complex pools of bacteria containing the pig genome partitioned into BACs. This genomic library contains about 97000 clones and covers the entire pig genome approximately 4.7 times. Initially, seventeen “primary pools” of bacteria which between them contain the entire genomic library are obtained from HGMP. These “primary pools” are screened by PCR using the primers described above. Any “primary pool” that produces a PCR product will contain somewhere within it the gene of interest. The “secondary pool” that contains the fifteen individual sub-pools that make up the “primary pool” are then screened by PCR. The sub-pool which produces a PCR product is then screened to identify the “tertiary pool” that contains the gene of interest. The “tertiary pool” comes in a 384 well format that can be screened by PCR in such a way that the individual BAC clone containing the gene of interest can be identified.

DNA is prepared from the BAC containing the gene of interest using a Qiagen “Midiprep” kit. The DNA is partially digested with a restriction enzyme that has a recognition sequence 6 base pairs long. This digest will produce a number of fragments of DNA that can be cloned into a plasmid and transformed into bacteria. This plasmid DNA can be grown up in separable populations of bacteria, purified and screened again using the same PCR primers used to identify the BAC. The plasmid DNA contains a fragment of DNA, which is preferably approximately 10 to 15 kilobase pairs long, which includes an exon in which it is desired to make a modification which will be introduced into the target gene to disrupt its function. For example, the modification may be a stop codon or a deletion as discussed herein. In some embodiments, the exon into which the modification is introduced is the first coding exon of the gene which is to be disrupted. The identified fragment is then extensively mapped using a combination of digestion with restriction enzymes and DNA sequencing. This fragment of DNA is then used to generate the modifications to be introduced into the target gene.

A gene identified as leading to the production of an antigenic determinant is disrupted by homologous recombination using any of “Positive/Negative”, “Gene Trap”, “Overlapping” constructs or a construct which inserts a stop codon in all three reading figures. Any of these methods may be used twice if one desires to disrupt both copies of the endogenous target sequence. The main modification is that for the positive/negative, gene trap and over lapping constructs, the second time these constructs are used to knockout a gene, the “positive” marker in each case should be distinguishable from the “positive” marker used in the constructs to knock out the first copy of the gene.

A number of different DNA construct designs can be used to distinguish homologous recombination from random integration, thereby facilitating the identification of cells in which the desired homologous recombination has occurred. Below, four exemplary types of DNA construct that can disrupt a genes function by homologous recombination are described in detail. The first three (“Positive/Negative selection constructs,” “Gene Trapping constructs,” and “Overlapping constructs”) all provide methods that allow homologous recombination to be efficiently distinguished from random integration.

Positive/Negative Knockout Construct

One type of construct used is a Positive/Negative Knockout Construct. In this construct a “positive” marker is one that indicates that the DNA construct has integrated somewhere in the genome. A “negative” marker is one that indicates that the DNA construct has integrated at random in the genome, (Hanson et al., “Analysis of biological selections for high-efficiency gene targeting,” Mol.Cell Biol. 15 (1):45-51 (1995); the disclosure of which is hereby incorporated by reference in its entirety).

The “positive” marker is a gene under the control of a constitutively active promoter, for example the promoters of Cyto MegaloVirus (CMV) or the promoter of Simian Virus 40 (SV40). The gene controlled in this way may be an auto-fluorescent protein such as, for example, Enhance Green Fluorescent Protein (EGFP) or DsRed2 (both from Clontech), a gene that encodes resistance to a certain antibiotic (neomycin resistance or hygromycin resistance), a gene encoding a cell surface antigen that can be detected using commercially available antibody, for example CD4 or CD8 (antibodies raised against these proteins come from Rockland, Pharmingen or Jackson), and the like.

The “negative” marker is also a gene under the control of a constitutively active promoter like that of CMV or SV40. The gene controlled in this way may also be an auto-fluorescent protein such as EGFP or DsRed2 (Clontech), a gene that encodes resistance to a certain antibiotic (neomycin resistance or hygromycin resistance) a gene encoding a cell surface antigen that can be detected by antibodies, and the like. However, the “negative” marker may also be a gene whose product either causes the cell to die by apoptosis, for example, or changes the morphology of the cell in such a way that it is readily detectable by microscopy, for example E-cadherin in early blastocysts.

The “positive” marker is flanked by regions of DNA homologous to genomic DNA. The region lying 5′ to the “positive” marker can be about 1 kB in length, to allow PCR analysis using the primers specific for the “positive” marker and a region of the genome that lies outside of the recombination construct, but may have any length which permits homologous recombination to occur. If the PCR reaction using these primers produces a DNA product of expected size, this is further evidence that a homologous recombination event has occurred. The region to the 3′ of the positive marker can also have any length which permits homologous recombination to occur. Preferably, the 3′ region is as long as possible, but short enough to clone in a bacterial plasmid. For example, the upper range for such a stretch of DNA can be about 10 kB in some embodiments. This 3′ flanking sequence can be at least 3 kB. To the 3′ end of this stretch of genomic DNA the “negative” marker is attached.

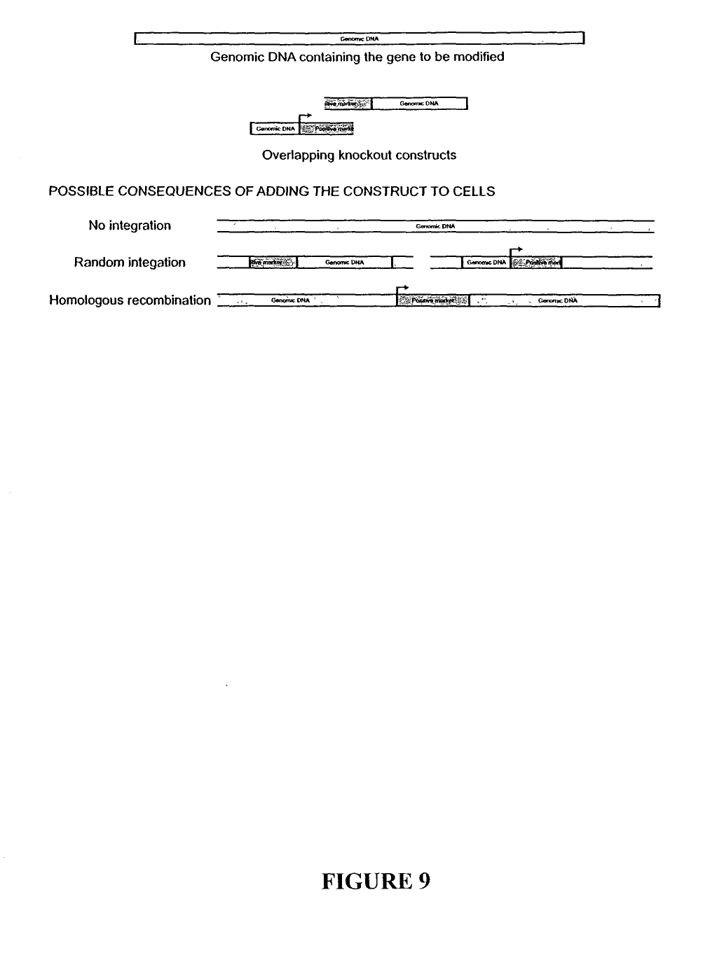

Once this DNA has been introduced into the cell, the cell will fall into one of three phenotypes: (1) No expression of either the “positive” or “negative” marker, for example, where there has been no detectable integration of the DNA construct. (2) Expression of the “positive” and “negative” markers. There may have been a random integration of this construct somewhere within the genome. (3) Expression of the “positive” marker but not the “negative” marker. Homologous recombination may have occurred between the genomic DNA flanking the “positive” marker in the construct and endogenous DNA. In this way the “negative” marker has been lost. These are the desired cells. These three possibilities are shown schematically in FIG. 9.

Gene Trapping Construct

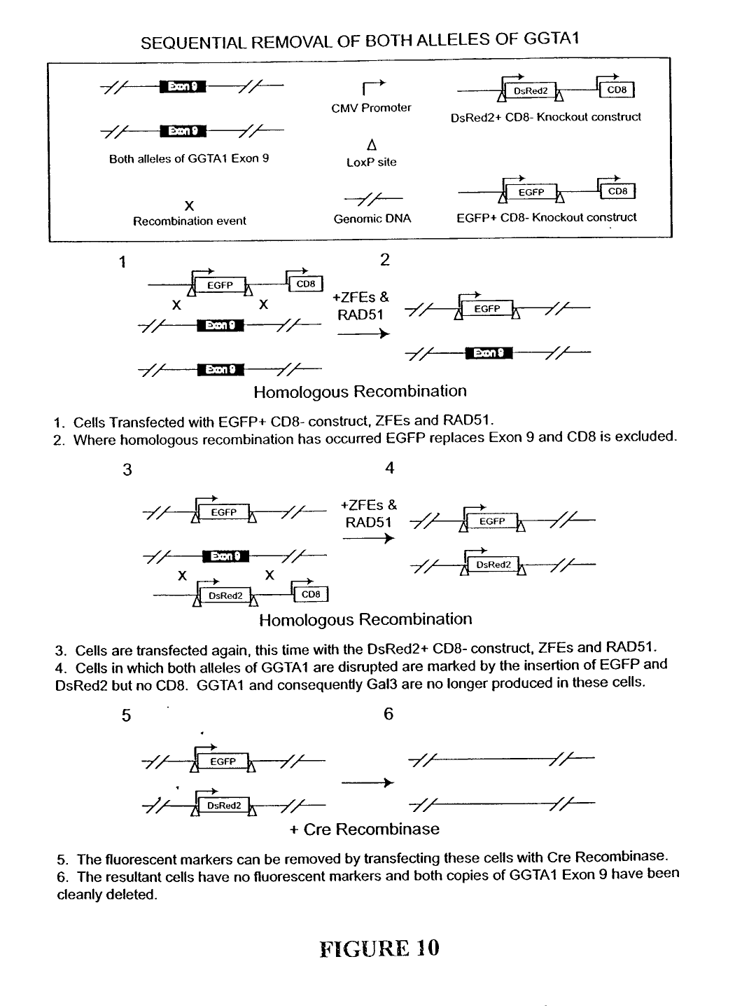

Another type of construct used is called a “Gene Trapping construct.” These constructs contain a promoter-less “positive” marker gene. This gene may be, for example, any of the genes mentioned above for a positive/negative construct. This marker gene is also flanked by pieces of DNA that are homologous to genomic DNA. In this case however, 5′ flanking DNA must put the marker gene under the control of the promoter of the gene to be modified if homologous recombination happens as desired (Sedivy et al., “Positive genetic selection for gene disruption in mammalian cells by homologous recombination,” Proc.Natl.Acad.Sci. U.S.A 86 (1):227-231 (1989); the disclosure of which is hereby incorporated by reference in its entirety). Preferably, this 5′ flanking DNA does not drive expression of the “positive” marker gene by itself. One possible way of doing this is to make a construct where the marker is in frame with the first coding exon of the target gene, but does not include the actual promoter sequences of the gene to be modified. It should be noted that, in preferred embodiments, this technique works if the gene to be modified is expressed at a detectable level in the cell type in which homologous recombination is being attempted. The higher the expression of the endogenous gene the more likely this technique is to work. The region 5′ to the marker can also have any length that permits homologous recombination to occur. Preferably, the 5′ region can be about 1 kB long, to facilitate PCR using primers in the marker and endogenous DNA, in the same way as described above. Similarly, preferably the 3′ flanking region can contain as long a region of homology as possible. An example of an enhancer trapping knockout construct is shown in FIG. 10.

These enhancer trapping based knockout constructs may also contain a 3′ flanking “negative” marker. In this case the DNA construct can be selected for on the basis of three criteria, for example. Expression of the “positive” marker under the control of the endogenous promoter, absence of the “negative” marker, and a positive result of the PCR reaction using the primer pair described above.

Over-lapping Knockout Construct

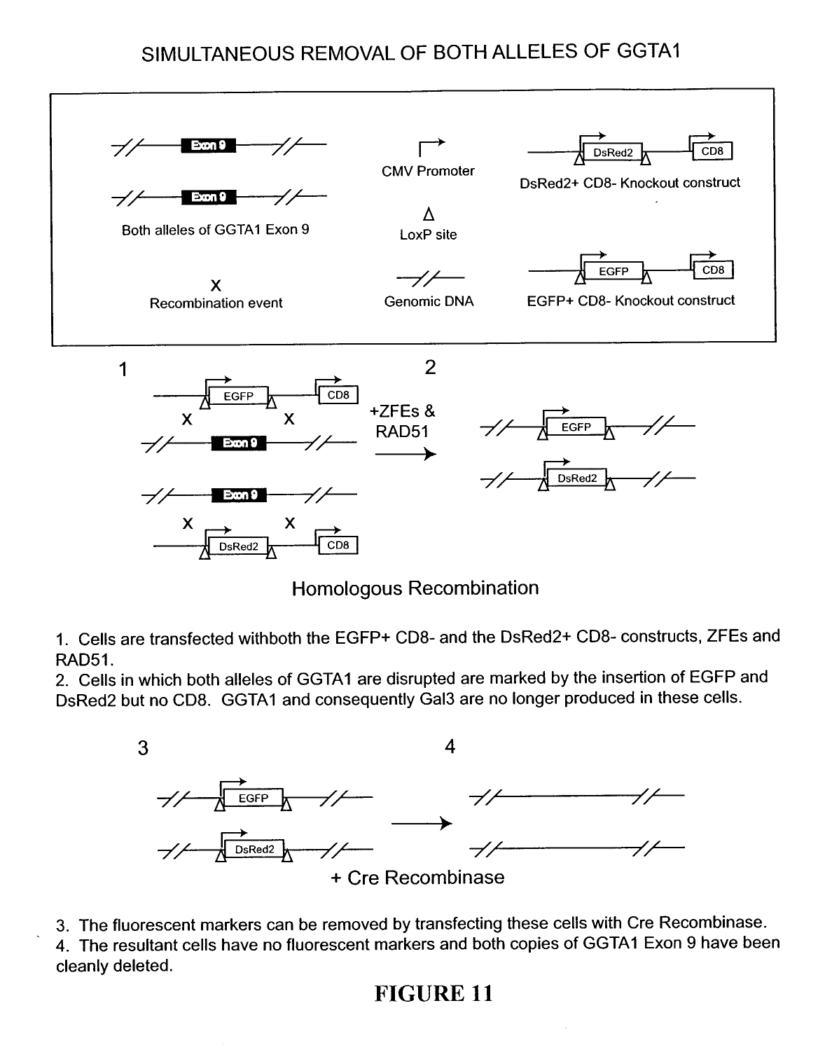

A further type of construct is called an “Over-lapping knockout construct”. This technique uses two DNA constructs (Jallepalli et al., “Securin is required for chromosomal stability in human cells,” Cell 105 (4):445-457 (2001), the disclosure of which is hereby incorporated by reference in its entirety). Each construct contains an overlapping portion of a “positive” marker, but not enough of the marker gene to make a functional reporter protein on its own. The marker is composed of both a constitutively active promoter, for example CMV or SV40 and the coding region for a “positive” marker gene, such as for example, any of those described above. In addition to the marker gene, each of the constructs contains a segment of DNA that flanks the desired integration site. The region of the gene replaced by the “positive” marker is the same size as that marker. If both of these constructs integrate into the genome in such a way as to complete the coding region for the “positive” marker, then that marker is expressed. The chances that both constructs will integrate at random in such an orientation are negligible. Generally, if both constructs integrate by homologous recombination, is it likely that a functional coding region for the “positive” marker will be recreated, and its expression detectable. An example of an overlapping knockout construct is shown in FIG. 11.

Another DNA construct, construct which inserts a stop codon in all three reading frames after homologous recombination does not contain an intrinsic means of distinguishing homologous recombination from random integration. Unlike the other constructs this one contains no marker genes either “positive” or “negative”. The construct is a stretch of DNA homologous to at least part of the coding region of a gene whose expression is to be removed. The only difference between this piece of DNA and its genomic homology is that somewhere in region of this DNA that would normally form part of the coding region of the gene, the following sequence, referred to herein as a “stopper” sequence, has been substituted: 5′-ACTAGTTAACTGATCA-3′ (SEQ ID NO. 10). This DNA sequence is 16 bp long, and its introduction via homologous recombination adds a stop codon in all three reading frames as well as a recognition site for SpeI and BclI. BclI is methylated by Dam and Dcm methylase activity in bacteria.

Integration by homologous recombination is detectable in two ways. The first method is the most direct, but it requires that the product of the gene being modified is expressed on the surface of the cell, and that there is an antibody that exists that recognizes this protein. If both of these conditions are met, then the introduction of the stop codons truncates the translation of the protein. The truncation shortens the protein so much that it is no longer functional in the cell or detectable by antibodies (either by FACS of Immuno-histochemistry). The second indirect way of checking for integration of the stopper sequence is PCR based. Primers are designed so that one lies outside of the knockout construct, and the other lies within the construct past the position of the stopper sequence. PCR will produce a product whether there has been integration or not. A SpeI restriction digest is carried out on the product of this PCR. If homologous recombination has occurred the stopper sequence will have introduced a novel SpeI site that should be detectable by gel electrophoresis.

Integration of any of the constructs described above by homologous recombination can be verified using a Southern blot. Introduction of the construct will add novel restriction endonuclease sites into the target genomic DNA. If this genomic DNA is digested with appropriate enzymes the DNA flanking the site of recombination is contained in fragments of DNA that are a different size compared to the fragments of genomic DNA which have been digested with the same enzymes but in which homologous recombination has not occurred. Radioactive DNA probes with sequences homologous to these flanking pieces of DNA can be used to detect the change in size of these fragments by Southern blotting using standard methods.

Using either the “Positive/negative”, “Gene Trap” or “Over-lapping” strategies described above, the genetically modified cell ends up with an exogenous marker gene integrated into the genome. In any of these strategies the marker gene and any exogenous regulatory sequences may be flanked by LoxP recombination sites and subsequently removed.

Removal occurs because recombination may occur between two LoxP sites which excises the intervening DNA (Sternberg et al., “Bacteriophage P1 site-specific recombination. II. Recombination between loxP and the bacterial chromosome,” J.Mol.Biol. 150 (4):487-507 (1981); and Sternberg et al., “Bacteriophage P1 site-specific recombination. I. Recombination between loxP sites,” J.Mol.Biol. 150 (4):467-486 (1981); the disclosures of which are both hereby incorporated by reference in their entireties). This recombination is driven by the Cre recombinase (Abremski et al., “Bacteriophage P1 site-specific recombination. Purification and properties of the Cre recombinase protein,” J.Biol.Chem. 259 (3):1509-1514 (1984); the disclosure of which is hereby incorporated by reference in its entirety). This can be provided in cells in which homologous recombination has occurred by introducing it into cells through lipofection (Baubonis et al., “Genomic targeting with purified Cre recombinase,” Nucleic Acids Res. 21 (9):2025-2029 (1993); the disclosure of which is hereby incorporated by reference in its entirety), or by transfecting the cells with a vector comprising an inducible promoter linked to DNA encoding Cre recombinase (Gu et al., “Deletion of a DNA polymerase beta gene segment in T cells using cell type-specific gene targeting,” Science 265 (5168):103-106 (1994); the disclosure of which is hereby incorporated by reference in its entirety).

It will be appreciated that rather than using a recombination vector comprising a disruption in the coding sequence of the target gene, the recombination vector may contain a sequence which introduces a deletion in the target gene or a sequence which disrupts the gene in some other manner, such as by disrupting the promoter from which transcription of the target gene initiates.

### Methods to Enhance the Rate of Homologous Recombination

There are two main methods for enhancing the rate of homologous recombination. Firstly, general recombination factors can be added to a cell, to increase the overall rate of recombination in the cell, both homologous and otherwise. Secondly, the introduction of double stranded DNA breaks at a particular position within the genome will enhance the rate of recombination at that genomic position.

Proteins that Enhance Homologous Recombination

The RecA system has been shown to increase the rate of homologous recombination in prokaryotes in Kowalczykowski et al., 1994, Microbiol. Rev. 58(3):401-465, the disclosure of which is incorporated herein by reference in its entirety may be used to enhance the frequency of homologous recombination events. Briefly, in this procedure the homologous recombination vector comprising the disrupted gene is contacted with RecA under conditions which permit RecA to bind to the sequence to be incorporated into the genome of the host organism. The sequence of the disrupted gene, which is coated with RecA, is then introduced into the cell in which the gene is to be disrupted as described above.

Alternatively, there are a number of proteins that enhance the rate of homologous recombination in higher eukaryotes. In mammals the principal one is Rad51 (reviewed in S. L. Gasior, H. Olivares, U. Ear, D. M. Hari, R. Weichselbaum, and D. K. Bishop. “Assembly of RecA-like recombinases: Distinct roles for mediator proteins in mitosis and meiosis.” Proc.Natl.Acad.Sci. U.S.A 98 (15):8411-8418, 2001). This has been cloned in both mice and humans. The nucleotide sequences of the mouse and the human Rad51 mRNA are available on Pubmed (hyper text transfer protocol: www4.ncbi.nlm.nih.gov/PubMed/), accession numbers (mouse [Acc # D13473.1] and human [Acc # XM_{—}007550], the disclosure of which are incorporated herein by reference in their entireties.).

There are a number of accessory proteins that are also involved (Reviewed in M. Modesti and R. Kanaar. “Homologous recombination: from model organisms to human disease.” Genome Biol. 2 (5):REVIEWS1014, 2001). Of these RPA (Replication Protein A) is extremely abundant in all cell types and is unlikely to be limiting. Accordingly, it may not be necessary to clone the gene encoding RPA for these methods. Other accessory proteins that may be limited and that may be cloned for use in these methods in addition to Rad51 include Rad52, Xrcc2, Xrcc3, Rad51B, Rad51C, Rad51D and any as yet unidentified factors that are directly proven to interact with Rad51 and consequently increase the rate of homologous recombination.

a) Cloning When the Sequence of the Gene is Already Published (Mouse and Human)

Published genes encoding proteins known to be important for homologous recombination are cloned. To illustrate how this will be done human Rad51 is used as an example. Using sequence data published online on Pubmed primers are designed to clone the coding region of this gene by PCR using cDNA produced by reverse transcription at tissue specific mRNA as a template. Rad51 is expressed at high levels in the cells of the body were there is a lot of recombination, for example hematopoeitic stem cells and germ cells. These tissues provide the best source of tissue to prepare cDNA from. Rad51 is present in most tissues of the body at lower levels, so these tissues can also be used (T. Morita, Y. Yoshimura, A. Yamamoto, K. Murata, M. Mori, H. Yamamoto, and A. Matsushiro. “A mouse homolog of the Escherichia coli recA and Saccharomyces cerevisiae RAD51 genes.” Proc.Natl.Acad.Sci. U.S.A 90 (14):6577-6580, 1993 and A. Shinohara, H. Ogawa, Y. Matsuda, N. Ushio, K. Ikeo, and T. Ogawa. “Cloning of human, mouse and fission yeast recombination genes homologous to RAD51 and recA.” Nat. Genet. 4 (3):239-243, 1993).

In the first instance PCR will be used to try and clone the entire coding region. It may be necessary to try a variety of strategies to produce a specific fragment of DNA. These strategies include “touch down” PCR in which the annealing temperature starts off very high and is reduced in subsequent cycles to increase specificity. “Nested” PCR uses concentric primer pairs to clone very rare transcripts. An “outer” primer pair is used followed by another round of PCR using a pair of “inner” primers specific for the same gene that lie within the region flanked by the outer primers. When such PCR yields a fragment of DNA of the expected size this is run on an agarose gel and gel purified using a kit such as “Qiaex II” (Qiagen). This DNA is then cloned into a vector designed to receive PCR products, for example pGEM-T Easy (Promega). A plasmid containing the PCR product is identified by miniprep and restriction digestion, this DNA is then sequenced.

If it proves impossible to clone the perfect full-length coding sequence in this way, then PCR may be used to clone a portion of the gene to use as a probe for filter screening a cDNA library. A probe is a region of sequence specific to the gene of interest, at least 200 bp long, but preferably longer. These primers are designed on the basis of those most likely to work in PCR using software such as Omiga 2.0 (Genetics Computer Group). When a fragment of DNA of an expected size is produced it is cloned and sequenced as described above.

Once a fragment of the gene of interest, for example Rad51, is identified, this fragment is cut out of the cloning vector, for example pGEM-T, using suitable restriction enzymes, usually Eco RI or Not I. The released fragment is gel purified. This DNA is then used to produce a P³² labeled radioactive DNA probe using a kit such as Rediprime II (Amersham Pharmacia Biotech). This radioactive DNA probe is then used on nitrose filters produced as a replica of bacteria containing a plasmid cDNA library plated out on selective agarose plates. These filters are soaked in a hybridization solution containing deionionsed water, herring sperm DNA, SDS (sodium dodecyl sulfate), dextran sulfate, formamide and SSC buffer (sodium citrate buffer) and the radioactive DNA probe. The following day the filters are washed with a series of buffers containing SDS and progressively lower concentrations of SSC buffer. The filters are then wrapped in Saran Wrap and left, in the dark, next to photographic film for 3 to 4 days at −80° C. The films are then developed in a photographic developer (such as those made by Kodak). The area of the bacterial plate that is identified in this way as one containing a positive clone is then re-plated at a lower density. This is then re-screened just as before. In this way a single bacterial clone containing the full-length gene of interest is identified.

b.) Cloning When a Gene Enhancing Homologous Recombination is not Cloned in the Species of Interest (Pig, Cow, Sheep, Rat etc.)

To enhance the rate of homologous recombination in another mammal in which Rad51 and its accessory proteins have not been cloned, for example in pigs, cows, sheep or rats, the species specific genes are cloned. Porcine Rad51 is used for the following example but the same methodology is directly applicable to any other species and any other factor that can increase the rate of homologous recombination.

The sequence of mouse [Acc # D13473.1] and the human [Acc # XM_{—}007550] Rad51 are available on Pubmed. These sequences are aligned using the software Omiga™ 2.0 (Genetics Computer Group) to identify areas of sequence identity. A pair of PCR primers are designed that lie within an area of identity between mouse and human homologs, that will produce a fragment of DNA large enough to use as a DNA probe for filter screening a cDNA library (as mentioned before this is at least 200 bp but the larger the better). One such primer pair for porcine Rad51 is the forward primer 5′-GAATTAGTGAAGCCAAAG -3′ (SEQ ID NO: 19) and the backward primer 5′-ACAATAAGCAGTGCATACC-3′ (SEQ ID NO: 20). PCR using these primers produces a product of 470 bp. The template is cDNA prepared from tissue specific RNA, in our example pig kidney and heart cDNA produced at Stell from a Yucatan minipig are used. The resultant PCR product is cloned and sequenced using standard methods. A comparison of the resultant sequence to the previously published sequences (usually mouse and human) will identify the likelihood that it is a homologous sequence. Below the pig fragment from our example, with the differences between this sequence and the mouse and human Rad51 sequences indicated. The large degree of identity makes this is very likely the homologous sequence.

The probe sequence derived for porcine RAD51

[image]

Such a fragment of DNA is used to screen a cDNA library in just the same way as that described above for the published human and mouse genes. In the pig example the cDNA library to be screened is the one produced at Stell from Yucatan minipig kidney mRNA. In order to test that this fragment is specific for a single gene it is used as a probe for a northern blot analysis of the target tissue's mRNA using standard methods. In our example the porcine Rad51 probe produces a single band when used to blot kidney Yucatan pig mRNA. Afterwards, this probe sequence of DNA is used to filter screen for the full length coding region of interest using exactly the same method described above for the previously sequenced genes.

Sequence Data for both the mouse and the human currently exists for these Rad51 accessory factors: Rad52 (mouse [Acc # NM_{—}011236], human [Acc # NM_{—}002879]), Xrcc2 (mouse [Acc # NM_{—}020570], human [Acc # Y08837]), Rad51C (mouse [Acc # NM_{—}052269], human (Acc # NM_{—}058216, NM_{—}002876, NM_{—}058217]) and Rad51D (mouse [Acc # AF034955], human [Acc # AF034956]). The sequences of the foregoing accession numbers are incorporated herein by reference in their entireties. All of these genes can be cloned using the method described above.

Sequence data only exists from a single species for the following accessory proteins: Xrcc3 (human [Ace # NM_{—}005432]) and Rad51B (human [Acc # U84138] The sequences of the foregoing accession numbers are incorporated herein by reference in their entireties). For these genes the regions that tend to be conserved within their family of genes are used to design primers to clone a probe with. The resultant PCR product will be aligned to the single previously cloned gene. Otherwise the method used to clone these genes is exactly the same as that described above.

Producing Protein from the Genes Encoding the Homologous Recombination Factors

Once the full-length coding regions for the recombination factors are cloned, they are cloned into the following types of expression vector: a mammalian expression vector such as the pcDNA3.1 vectors produced by Invitrogen, a bacterial expression and purification vector such as pMal-c2x (New England Biolabs) or a vector from which the gene can be transcribed and translated in a cell-free lysate system. For example, any vector in which the gene's expression can be driven by the T7, T3 or SP6 polymerase can be used in the TNT reticulocyte lysate system (Promega), for example pcDNA3.1 (Invitrogen) is suitable.

The mammalian expression vector containing the coding region for the gene of interest may then be transfected into mammalian cells, either primary cells or cell lines such as HeLa cells. This vector will then produce homologous recombination factors using the cell's own transcriptional and translational machinery. This may be done in combination with other proteins and DNA constructs that are designed to cause homologous recombination.

As an alternative to this approach, homologous recombination proteins may be produced in bacteria or a cell free system such as TNT Reticulocyte Lysate. These proteins are then purified by means of an associated peptide tag (including maltose binding protein, a myc epitope and a histidine tag) and are then quantified. A known concentration of this purified protein is then microinjected into the primary cell or cell line, either alone or in combination with other proteins and DNA constructs.

Introducing Double Stranded DNA Breaks Within a Gene to Enhance Disruption of that Gene by Homologous Recombination.

In some embodiments, the frequency of homologous recombination at or near the endogenous nucleotide sequence is enhanced by cleaving the endogenous nucleotide sequence in the cell with an endonuclease. Preferably, both strands of the endogenous nucleotide sequence are cleaved by the endonuclease. A nucleic acid comprising a nucleotide sequence homologous to at least a portion of the chromosomal region containing or adjacent to the endogenous nucleotide sequence at which the endonuclease cleaves is introduced into the cell such that homologous recombination occurs between the nucleic acid and the chromosomal target sequence. Thereafter, a cell in which the desired homologous recombination event has occurred may be identified and used to generate a genetically modified organism using techniques such as nuclear transfer.

In some embodiments, the frequency of homologous recombination is enhanced using the method described in Cohen-Tannoudji et al., 1998, Mol. Cell. Biol. 18(3):1444-1448, the disclosure of which is incorporated herein by reference in its entirety. Briefly, this strategy induces an endogenous gap repair process at a defined location within the genome by induction of a double-stranded break in the gene to be disrupted. In turn, the double-stranded break increases the frequency of recombination. Double-stranded breaks are introduced into the chromosomal target genes by introducing an I-SceI yeast meganuclease restriction site into the chromosomal target genes in the donor cells. Thereafter, I-SceI yeast meganuclease is introduced into the cells using a transient expression vector and the homologous recombination vector bearing the disrupted target gene is also introduced into the donor cells.

In preferred embodiments of the present invention, zinc finger endonucleases (ZFEs) are used to enhance the rate of homologous recombination in cells. Preferably, the cells are from species in which totipotent stem cells are not available, but in other embodiments the cells may be from an organism in which totipotent stem cells are available, and, in some embodiments, the cell may be a totipotent stem cell. Preferably, the cell is a primary cell, but in some embodiments, the cell may be a cell from a cell line. For example, in some embodiments, the cells may be from an organism such as a mammal, a marsupial, a teleost fish, an avian and the like. The mammal may be a human, a non-human primate, a sheep, a goat, a cow, a rat, a pig, and the like. In other embodiments, the mammal can be a mouse. In some embodiments, the teleost fish may be a zebrafish. In other embodiments the avian may be a chicken, a turkey, and the like.

The cells may be any type of cell which is capable of being used to generate a genetically modified organism or tissue. For example, in some embodiments, the cell may be primary skin fibroblasts, granulosa cells, primary fetal fibroblasts, stem cells, germ cells, fibroblasts or non-transformed cells from any desired organ or tissue.

In some embodiments of the present invention, a ZFE is used to cleave an endogenous chromosomal nucleotide sequence at or near which it is desired to introduce a nucleic acid by homologous recombination. The ZFE comprises a zinc finger domain which binds near the endogenous nucleotide sequence at which is to be cleaved and an endonuclease domain which cleaves the endogenous chromosomal nucleotide sequence. As mentioned, above, cleavage of the endogenous chromosomal nucleotide sequence increases the frequency of homologous recombination at or near that nucleotide sequence. In some embodiments, the ZFEs can also include a purification tag which facilitates the purification of the ZFE.

Any suitable endonuclease domain can be used to cleave the endogenous chromosomal nucleotide sequence. The endonuclease domain is fused to the heterologous DNA binding domain (such as a zinc finger DNA binding domain) such that the endonuclease will cleave the endogenous chromosomal DNA at the desired nucleotide sequence. As discussed below, in some embodiments the endonuclease domain can be the HO endonuclease. In more preferred embodiments the endonuclease domain may be from the Fok I endonuclease. One of skill in the art will appreciate that any other endonuclease domain that is capable of working with heterologous DNA binding domains, preferably with zinc finger DNA binding domains, can be used.

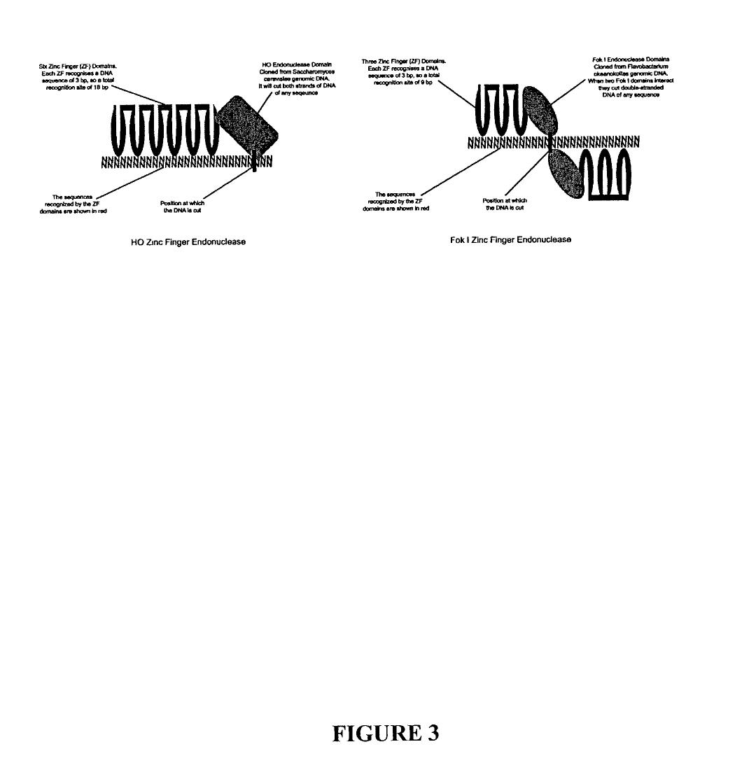

The HO endonuclease domain from Saccharomyces cerevisiae is encoded by a 753 bp Pst I-Bgl II fragment of the HO endonuclease cDNA available on Pubmed (Acc # X90957, the disclosure of which is incorporated herein by reference in its entirety). The HO endonuclease cuts both strands of DNA (Nahon et al., “Targeting a truncated Ho-endonuclease of yeast to novel DNA sites with foreign zinc fingers,” Nucleic Acids Res. 26 (5):1233-1239 (1998); the disclosure of which is incorporated herein by reference in its entirety). FIG. 3 illustrates the sequence of the Pst I-Bgl II fragment of the HO endonuclease cDNA which may be used in the ZFEs of the present invention. Saccharomyces cerevisiae genes rarely contain any introns, so, if desired, the HO gene can be cloned directly from genomic DNA prepared by standard methods. For example, if desired, the HO endonuclease domain can be cloned using standard PCR methods.

In some embodiments, the Fok I (Flavobacterium okeanokoites) endonuclease may be fused to a heterologous DNA binding domain. The Fok I endonuclease domain functions independently of the DNA binding domain and cuts a double stranded DNA only as a dimer (the monomer does not cut DNA) (Li et al., “Functional domains in Fok I restriction endonuclease,” Proc.Natl.Acad.Sci.U.S.A 89 (10):4275-4279 (1992), and Kim et al., “Hybrid restriction enzymes: zinc finger fusions to Fok I cleavage domain,” Proc.Natl.Acad.Sci.U.S.A 93 (3):1156-1160 (1996); the disclosures of which are incorporated herein by reference in their entireties). Therefore, in order to create double stranded DNA breaks, two ZFEs are positioned so that the Fok I domains they contain dimerise.

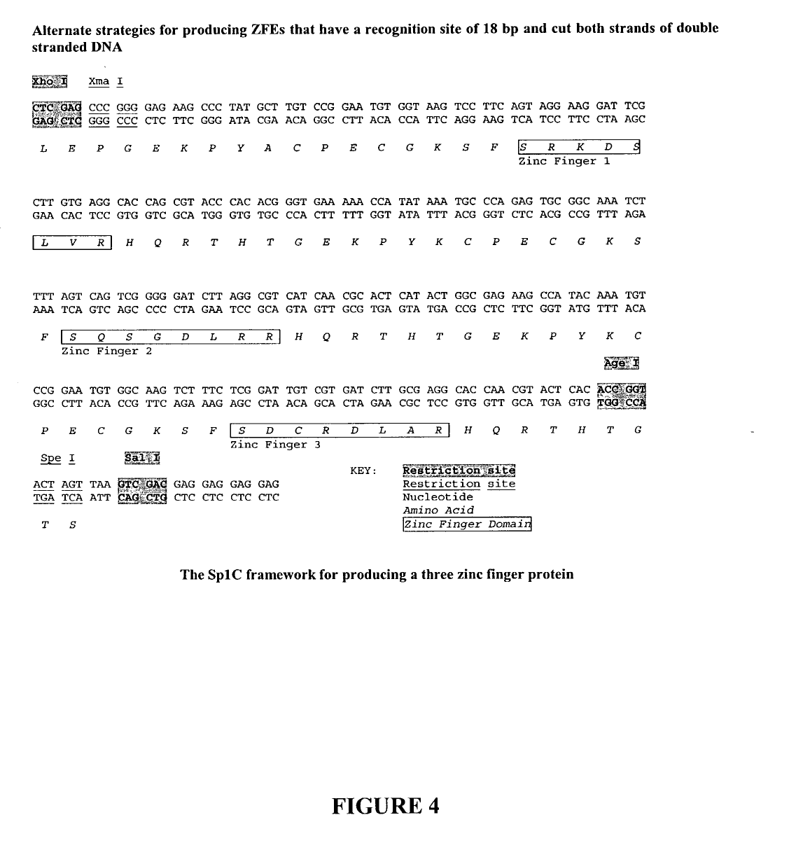

The Fok I endonuclease domain can be cloned by PCR from the genomic DNA of the marine bacteria Flavobacterium okeanokoites (ATCC) prepared by standard methods. The sequence of the Fok I endonuclease is available on Pubmed (Acc # M28828 and Acc # J04623, the disclosures of which are incorporated herein by reference in their entireties). FIG. 4 depicts the sequence of the Fok I endonuclease domain that can be used in chimeric endonucleases such as those utilized in the present methods.

Again, it will be appreciated that any other endonuclease domain that works with heterologous DNA binding domains can be fused to the zinc finger DNA binding domain.

As mentioned above, the ZFE includes a zinc finger domain with specific binding affinity for a desired specific target sequence. In preferred embodiments, the ZFE specifically binds to an endogenous chromosomal DNA sequence. The specific nucleic acid sequence or more preferably specific endogenous chromosomal sequence can be any sequence in a nucleic acid region where it is desired to enhance homologous recombination. For example, the nucleic acid region may be a region which contains a gene in which it is desired to introduce a mutation, such as a point mutation or deletion, or a region into which it is desired to introduce a gene conferring a desired phenotype.

There are a large number of naturally occurring zinc finger DNA binding proteins which contain zinc finger domains that may be incorporated into a ZFE designed to bind to a specific endogenous chromosomal sequence. Each individual “zinc finger” in the ZFE recognizes a stretch of three consecutive nucleic acid base pairs. The ZFE may have a variable number of zinc fingers. For example, ZFEs with between one and six zinc fingers can be designed. In other examples, more than six fingers can be used. A two finger protein has a recognition sequence of six base pairs, a three finger protein has a recognition sequence of nine base pairs and so on. Therefore, the ZFEs used in the methods of the present invention may be designed to recognize any desired endogenous chromosomal target sequence, thereby avoiding the necessity of introducing a cleavage site recognized by the endonuclease into the genome through genetic engineering

In preferred embodiments the ZFE protein can be designed and/or constructed to recognize a site which is present only once in the genome of a cell. For example, one ZFE protein can be designed and made with at least five zinc fingers. Also, more than one ZFE protein can be designed and made so that collectively the ZFEs have five zinc fingers (i.e. a ZFE having two zinc fingers may complex with a ZFE having 3 zinc fingers to yield a complex with five zinc fingers). Five is used here only as an exemplary number. Any other number of fingers can be used. Five zinc fingers, either individually or in combination, have a recognition sequence of at least fifteen base pairs. Statistically, a ZFE with 5 fingers will cut the genome once every 4¹⁵ (about 1×10⁹) base pairs, which should be less than once per average size genome. In more preferred embodiments, an individual protein or a combination of proteins with six zinc fingers can be used. Such proteins have a recognition sequence of 18 bp.

Appropriate ZFE domains can be designed based upon many different considerations. For example, use of a particular endonuclease may contribute to design considerations for a particular ZFE. As an exemplary illustration, the yeast HO domain can be linked to a single protein that contains six zinc fingers because the HO domain cuts both strands of DNA. Further discussion of the design of sequence specific ZFEs is presented below.

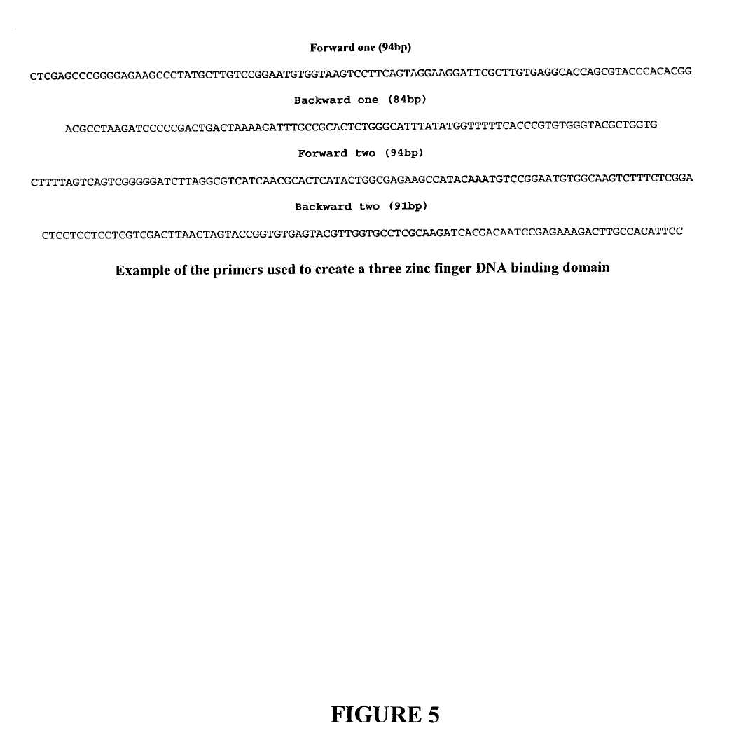

Alternatively, the Fok I endonuclease domain only cuts double stranded DNA as a dimer. Therefore, two ZFE proteins can be made and used in the methods of the present invention. These ZFEs can each have a Fok I endonuclease domain and a zinc finger domain with three fingers. They can be designed so that both Fok I ZFEs bind to the DNA and dimerise. In such cases, these two ZFEs in combination have a recognition site of 18 bp and cut both strands of DNA. FIG. 5 illustrates examples of a ZFE that includes an HO endonuclease, and ZFEs using the Fok I endonuclease. Each ZFE in FIG. 5 has an 18 bp recognition site and cuts both strands of double stranded DNA.

The particular zinc fingers used in the ZFE will depend on the target sequence of interest. A target sequence in which it is desired to increase the frequency of homologous recombination can be scanned to identify binding sites therein which will be recognized by the zinc finger domain of a ZFE. The scanning can be accomplished either manually (for example, by eye) or using DNA analysis software, such as MacVector™ (Macintosh) or Omiga 2.0™ (PC), both produced by the Genetics Computer Group. For a pair of Fok I containing ZFEs, two zinc finger proteins, each with three fingers, bind DNA in a mirror image orientation, with a space of 6 bp in between the two. For example, the sequence that is scanned for can be 5′-G/A N N G/A N N G/A N N N N N N N N N N C/T N N C/T N N C/T-3′(SEQ ID NO. ______ ). If a six finger protein with an HO endonuclease domain attached is used, then the desired target sequence can be 5′-G/A N N G/A N N G/A N G/A N N G/A N N N G N N G/A N N-3′ (SEQ ID NO. ______ ), for example. In these examples, if “N” is any base pair, then all of the zinc fingers that bind to any sequence “GNN” and “ANN” are already determined (Segal et al., “Toward controlling gene expression at will: selection and design of zinc finger domains recognizing each of the 5′-GNN-3′ DNA target sequences,” Proc.Natl.Acad.Sci. U.S.A 96 (6):2758-2763 (1999), and Dreier et al., “Development of zinc finger domains for recognition of the 5′-ANN-3′ family of DNA sequences and their use in the construction of artificial transcription factors,” J.Biol.Chem. 276 (31):29466-29478 (2001); the disclosure of which are incorporated herein by reference in their entireties).

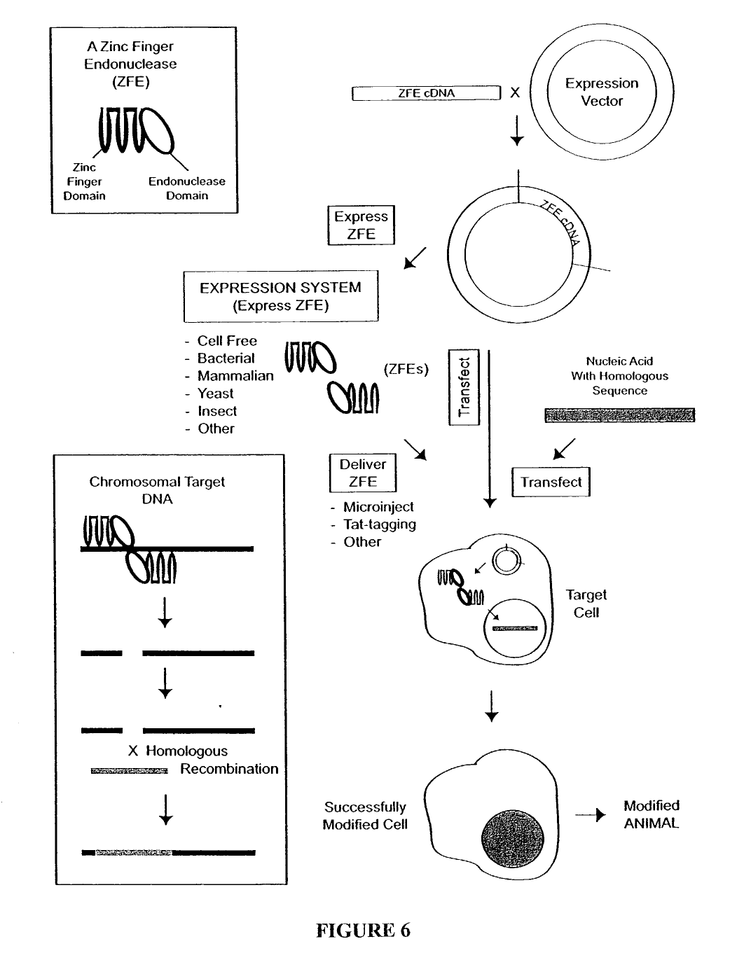

The sequence encoding the identified zinc fingers can be cloned into a vector according well known methods in the art. In one example, FIG. 6 illustrates one possible peptide framework into which any three zinc fingers that recognize consecutive base pair triplets can be cloned. Any individual zinc finger coding region can be substituted at the positions marked for zinc finger 1, zinc finger 2 and zinc finger 3. In this particular example zinc finger 1 recognizes “GTG”, zinc finger 2 “GCA” and zinc finger 3 “GCC”, so all together this protein will recognize “GTGGCAGCC” (SEQ ID NO. 14). Restriction sites are present on either side of this sequence to facilitate cloning. The backbone peptide in this case is that of Sp1C, a consensus sequence framework based on the human transcription factor Sp1 (Desjarlais et al., “Use of a zinc-finger consensus sequence framework and specificity rules to design specific DNA binding proteins,” Proc.Natl.Acad.Sci. U.S.A 90 (6):2256-2260 (1993); the disclosure of which is incorporated herein by reference in its entirety).

Sp1C is a three finger network and as such can be the zinc finger DNA binding domain that is linked to the Fok I endonuclease domain. Using the restriction sites Age I and Xma I two three-finger coding regions can be joined to form a six-finger protein with the same consensus linker (TGEKP) between all fingers. This technique is described in (Desjarlais et al., “Use of a zinc-finger consensus sequence framework and specificity rules to design specific DNA binding proteins,” Proc.Natl.Acad.Sci. U.S.A 90 (6):2256-2260 (1993); the disclosure of which is incorporated herein by reference in its entirety.) This six finger framework can be the zinc finger DNA binding domain that is linked to a desired endonuclease domain. The skilled artisan will appreciate that many other frameworks can be used to clone sequences encoding a plurality of zinc fingers.

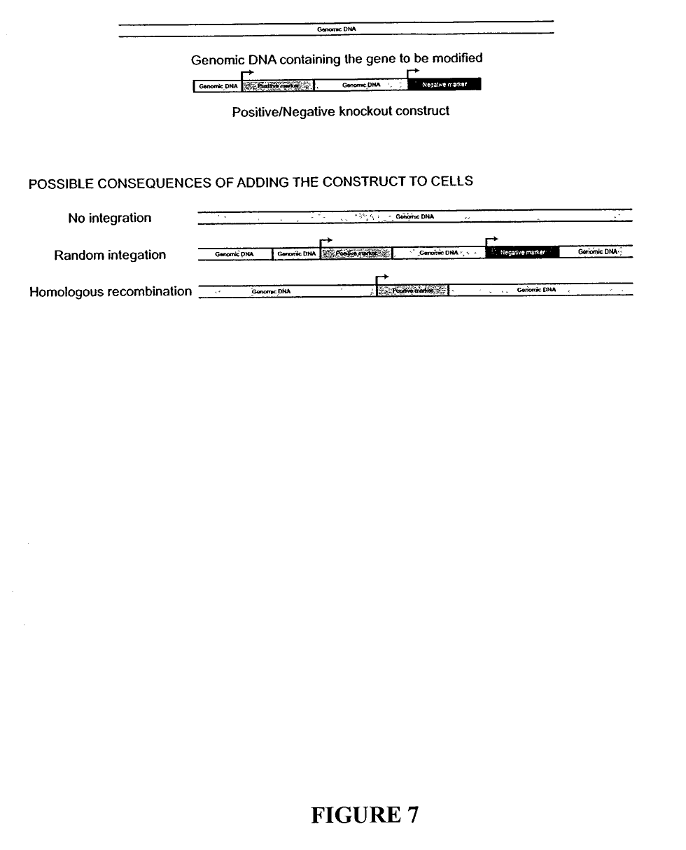

The sequence in FIG. 6 can be constructed using standard PCR methods. FIG. 7 illustrates exemplary PCR primers that can be used. Two 94 bp “forward” primers can encode the 5′ strand, and two “backward” primers that overlap these “forward” primers, one 84 bp the other 91 bp, can encode the 3′ strand. These primers can provide both the primers and the template when mixed together in a PCR reaction.

It will be appreciated that the zinc fingers in the ZFEs used in the methods of the present invention may be any combination of zinc fingers which recognize the desired binding site. The zinc fingers may come from the same protein or from any combination of heterologous proteins which yields the desired binding site.

A nucleotide sequence encoding a ZFE with the desired number of fingers fused to the desired endonuclease is cloned into a desired expression vector. There are a number of commercially available expression vectors into which the nucleotide sequence encoding the ZFE can be cloned. The expression vector is then introduced into a cell capable of producing an active ZFE. For example, the expression vector may be introduced into a bacterial cell, a yeast cell, an insect cell or a mammalian cell. Preferably, the cell lacks the binding site recognized by the ZFE. Alternatively, the cell may contain the binding site recognized by the ZFE but the site may be protected from cleavage by the endonuclease through the action of cellular enzymes.

In other embodiments, the ZFE can be expressed or produced in a cell free system such as TNT Reticulocyte Lysate. The produced ZFE can be purified by any appropriate method, including those discussed more fully herein. In preferred embodiments, the ZFE also includes a purification tag which facilitates purification of the ZFE. For example, the purification tag may be the maltose binding protein, myc epitope, a poly-histidine tag, HA tag, FLAG-tag, GST-tag, or other tags familiar to those skilled in the art. In other embodiments, the purification tag may be a peptide which is recognized by an antibody which may be linked to a solid support such as a chromatography column.

Many commercially available expression systems include purification tags, which can be used with the embodiments of the invention. Three examples of this are pET-14b (Novagen) which produces a Histidine tagged protein produced under the control of T7 polymerase. This vector is suitable for use with TNT Reticulocyte Lysate (Promega). The pMal system (New England Biolabs) which produces maltose binding protein tagged proteins under the control of the malE promoter in bacteria may also be used. The pcDNA vectors (Invitrogen) which produce proteins tagged with many different purification tags in a way that is suitable for expression in mammalian cells may also be used.

The ZFE produced as described above is purified using conventional techniques such as a chromatography column containing moieties thereon which bind to the purification tag. The purified ZFE is then quantified and the desired amount of ZFE is introduced into the cells in which it is desired to enhance the frequency of homologous recombination. The ZFE may be introduced into the cells using any desired technique. In a preferred embodiment, the ZFE is microinjected into the cells.

Alternatively, rather than purifying the ZFE and introducing it into the cells in which it is desired to enhance the frequency of homologous recombination, the ZFE may be expressed directly in the cells. In such embodiments, an expression vector containing a nucleotide sequence encoding the ZFE operably linked to a promoter is introduced into the cells. The promoter may be a constitutive promoter or a regulated promoter. The expression vector may be a transient expression vector or a vector which integrates into the genome of the cells.

As discussed above, a recombination vector comprising a 5′ region homologous to at least a portion of the chromosomal region in which homologous recombination is desired and a 3′ region homologous to at least a portion of the chromosomal region in which homologous recombination is introduced into the cell containing the ZFE. The lengths of the 5′ region and the 3′ region may be any lengths which permit homologous recombination to occur. The recombination vector also contains an insertion sequence located between the 5′ region and the 3′ region. The insertion sequence is a sequence which is desired to be introduced into the genome of the cell. Introduction of the insertion sequence into the genome of the cell disrupts a gene encoding a polypeptide comprising an antigenic determinant which is recognized by the recipient organism.

In some embodiments, the insertion sequence introduces a point mutation into the target endogenous chromosomal gene after homologous recombination has occurred. The point mutation disrupts the endogenous chromosomal gene.

In other embodiments, the insertion sequence introduces a deletion into an endogenous chromosomal gene after homologous recombination has occurred. In such embodiments, the insertion sequence may “knock out” the target gene.

In some embodiments, it may be desired to disrupt or knock-out both chromosomal copies of the target gene. In such embodiments, two homologous recombination procedures are performed as described herein to disrupt both copies of the chromosomal target sequence. Alternatively, a genetically modified organism in which one copy of the chromosomal target sequence has been disrupted desired may be generated using the methods described herein. Subsequently, cells may be obtained from the genetically modified organism and subjected to a second homologous recombination procedure as described herein. The cells from the second homologous recombination procedure may then be used to generate an organism in which both chromosomal copies of the target sequence have been disrupted as desired.

In some embodiments, the insertion sequence or a portion thereof may be located between two sites, such as loxP sites, which allow the insertion sequence or a portion thereof to be deleted from the genome of the cell at a desired time. In embodiments in which the insertion sequence or a portion thereof is located between loxP sites, the insertion sequence or portion thereof may be removed from the genome of the cell by providing the Cre protein. Cre may be provided in the cells in which a homologous recombination event has occurred by introducing Cre into the cells through lipofection (Baubonis et al., 1993, Nucleic Acids Res. 21:2025-9, the disclosure of which is incorporated herein by reference in its entirety), or by transfecting the cells with a vector comprising an inducible promoter operably linked to a nucleic acid encoding Cre (Gu et al., 1994, Science 265:103-106; the disclosure of which is incorporated herein by reference in its entirety).

In some embodiments, the recombination vector comprises a nucleotide sequence which encodes a detectable or selectable marker which facilitates the identification or selection of cells in which the desired homologous recombination event has occurred. For example, the detectable marker may be a cell surface protein which is recognized by an antibody such that cells expressing the cell surface marker may be isolated using FACS. Alternatively, the recombination vector may comprise a selectable marker which provides resistance to a drug.

The recombination vector may be introduced into the cell concurrently with the ZFE, prior to the ZFE, or after the ZFE. Cleavage of the chromosomal DNA by the ZFE enhances the frequency of homologous recombination by the recombination vector. Cells in which the desired recombination event has occurred are identified and, if desired, the chromosomal structure of the cells may verified using techniques such as PCR or Southern blotting. Further discussion of recombination vectors and methods for their use is provided in Example 9F, and several exemplary constructs are provided in FIGS. 9-11.

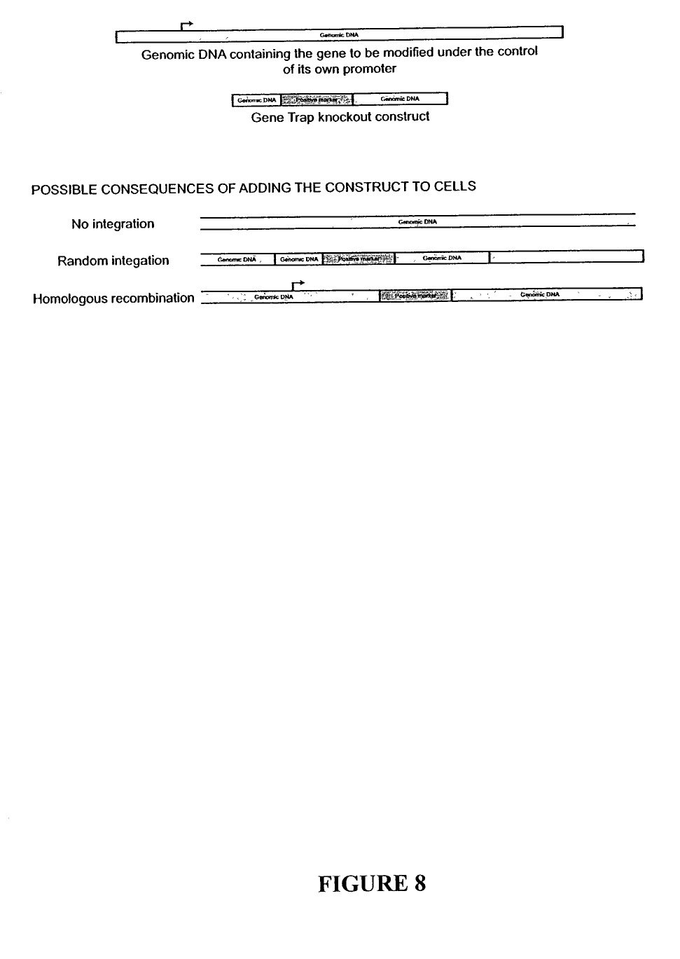

FIG. 8 illustrates a method of the present invention.

### Example 9A

### Design of a Zinc Finger Endonuclease

A ZFE is designed with an endonuclease domain that cuts DNA and a zinc finger domain which recognizes the specific DNA sequence “GTGGCAGCC” (SEQ ID NO: 22). The zinc finger domains encoded by the sequence illustrated in FIG. 6 are fused to the Fok I endonuclease.

A standard PCR protocol is performed using the primers illustrated in FIG. 7 in order to make and amplify the zinc finger domain encoded by the sequence in FIG. 6. The Fok I sequence illustrated in FIG. 4 is amplified using standard PCR methods. The amplified zinc finger domain sequence is joined to the amplified Fok I construct thereby forming a chimeric DNA sequence.

### Example 9B

### Design of 6-mer Endonuclease Domain

The zinc finger coding domains of FIG. 6 are cut using the restriction sites Age I and Xma I. The two three-finger coding domains are joined to form a six-finger coding domain with the same consensus linker (TGEKP) between all fingers. This six finger framework is linked to the HO endonuclease domain illustrated in FIG. 3.

### Example 9C

### Design of a Sequence Specific ZFE

A target endogenous chromosomal nucleotide sequence at or near which it is desired to enhance the frequency of homologous recombination is identified and scanned to identify a sequence which will be bound by a zinc finger protein comprising 6 zinc finger domains. If “N” is any base pair, then the zinc fingers are selected to bind to the following sequence within the target nucleic acid: 5′-G/A N N G/A N N G/A N N G/A N N G/A N N G/A N N-3′, where N is A, G, C or T (SEQ ID NO: 23).

### Example 9D

### Design of a Sequence Specific ZFE

A target endogenous chromosomal target sequence at or near which it is desired to enhance the frequency of homologous recombination is identified and scanned to identify a nucleotide sequence which will be recognized by a ZFE. Two 3-mer zinc finger domains for use with the Fok I endonuclease are designed by determining a zinc finger protein that will specifically bind to the target DNA in a mirror image orientation, with a space of 6 bp in between the two. If “N” is A, G, C or T, then all of the zinc fingers that bind to any sequence “GNN” and “ANN” are known. The zinc finger domain is selected to bind to the sequence 5′-G/A N N G/A N N G/A N N N N N N N N N N C/T N N C/T N N C/T-3′ (SEQ ID NO: 24).

### Example 9E

### Expression of the ZFE

The construct of Example 9A or 9B is introduced into the pMal bacterial expression vector (New England Biolabs) and expressed. The ZFE protein is expressed under the control of the malE promoter in bacteria tagged with a maltose binding protein. The ZFE protein is purified by maltose chromatography and quantified.

### Example 9F

### Generation of a Pig Cell in Which Both Chromosomal Copies of a Target Gene are Disrupted

ZFE protein from Example 9E is microinjected into a primary pig cell. A range of concentrations of ZFE protein is injected. In some embodiments, this range is approximately 5-10 mg of protein per ml of buffer injected, but any concentration of ZFE which is sufficient to enhance the frequency of homologous recombination may be used. Also, a recombination vector containing the target gene or a portion thereof in which the coding sequence has been disrupted is introduced into the pig cell. In some embodiments, the vector is introduced at a concentration of about 100 ng/μl, but any concentration which is sufficient to permit homologous recombination may be used. Both the DNA and the ZFE protein are resuspended in a buffer, such as 10 mM HEPES buffer (pH 7.0) which contains 30 mM KCl. The homologous recombination construct containing the disrupted coding sequence is either introduced into the cell by microinjection with the ZFE protein or using techniques such as lipofection or calcium phosphate transfection.

Homologous recombination is the exchange of homologous stretches of DNA. In order to alter the genome by homologous recombination, DNA constructs containing areas of homology to genomic DNA are added to a cell. One challenge associated with homologous recombination is that it normally occurs rarely. A second problem is that there is a relatively high rate of random integration into the genome. (Capecchi, “Altering the genome by homologous recombination,” Science 244 (4910):1288-1292 (1989); the disclosure of which is hereby incorporated by reference in its entirety). The inclusion of ZFEs increases the rate of homologous recombination while the rate of random integration is unaffected.

### Example 9G

### Construction of a Vector for Disrupting the GGTA1 Gene and Vectors Encoding ZFEs

Specifically a portion of the genomic region containing GGTA1 was isolated. A BAC was identified by PCR using a combination of primers specific for the largest coding exon, Exon 9. This BAC was digested with a range of commonly occurring restriction endonuclease (New England Biolabs). This was then probed as a Southern Blot. The probe used was the entire coding region of GGTA1. This showed that a 7.5 kB fragment of DNA flanked by EcoRI sites contains at lease part of the coding region of GGTA1. This fragment of DNA was gel purified and probed with a series of PCR primers. This analysis indicated that the 7.5 kB EcoRI fragment contained Exon 9 of GGTA1, this was later confirmed by sequencing. Detailed restriction mapping showed that the 7.5 kB of genomic DNA contained the entire Exon 9, and that Exon 9 is flanked by a Sac I and a Xba I restriction site, that are otherwise unique within the 7.5 kB. These restriction sites were used to clone in positive selection markers, or a region of DNA in which contains a “Stelloplast” within Exon 9. It has been shown previously in mice that removing Exon 9 of GGTA1 is enough to produce a functionally null mutation (R. G. Tearle, M. J. Tange, Z. L. Zannettino, M. Katerelos, T. A. Shinkel, B. J. Van Denderen, A. J. Lonie, I. Lyons, M. B. Nottle, T. Cox, C. Becker, A. M. Peura, P. L. Wigley, R. J. Crawford, A. J. Robins, M. J. Pearse, and A. J. d'Apice. The alpha-1,3-galactosyltransferase knockout mouse. Implications for xenotransplantation. Transplantation 61 (1):13-19, 1996).

In addition to this a 400 bp fragment of genomic DNA that lies 5′ to Exon 9 was cut off the end of the 7.5 kB EcoRI fragment and sequenced. This sequence is used to design a PCR primer that will be used to check that homologous recombination has occurred. The other primer for the PCR will lie either in the genomic region of DNA removed, the positive marker that replaces that piece of DNA or in alterations introduced into the DNA such as the construct which inserts a stop codon in all three reading frames after homologous recombination.

The initial strategy to knock out Exon 9 of GGTA1 is to use a Positive/Negative selection based strategy. In this specific case the positive markers used were DsRed2 (a red fluorescent protein from Clontech) and EGFP (a green fluorescent protein also from Clontech). Two positive markers are used, one for each of the two alleles that need to be removed for a fully function null. Using standard molecular cloning methods these markers are cloned in such a way that they are flanked by LoxP sites, the CMV promoter will drive their expression and a SV40 PolyA tail is added to their 3′ ends. All of this is flanked with Sac I and Spe I sites that facilitate cloning into the Sac I and Xba I sites surrounding Exon 9 of GGTA1. Cutting DNA with Spe I produces a sticky end compatible with that of DNA cut with Xba I. However, ligating these two DNAs together destroys both sites. Before cloning into the fragment of genomic DNA containing GGTA1 these markers were checked by sequencing and transfection into HeLa cells, where it was shown fluorescent protein is produced. The negative marker used for these knockout constructs is the human CD8 alpha chain, flanked by Sal I and Xho I sites, under the control of the SV40 promoter with a SV40 polyA tail. This is cloned to one end of the knockout construct into a Xho I site.

The second strategy that was used was to introduce the “Stelloplast” sequence has into the genomic DNA encoding GGTA1 Exon 9 in the using the Chameleon Double-Stranded Mutagenesis kit from Stratagene, using standard methods. The Sac I-Xba I fragment containing Exon 9 was subcloned into pBSK II (+) (Stratagene). Then kit was used following the manufacturer's instructions with the following mutagenic primers: to remove the Xho I from pBSK II (+) 5′-CCGTCGACCTGGAGGGGGGGC-3′ and to introduce the “Stelloplast” sequence into Exon 9 5′-GGAGGAGTTCTAGATAACTGATCATACATACTTCATGG-3′ (SEQ ID NO: 25). Both of these plasmids are 5′ phosphorulated. The presence of the “Stelloplast” mutation was checked by transforming resultant plasmids into a Dam methylase minus background and digesting with Bcl I.

The following ZFEs were constructed to enhance homologous recombination within the Exon 9 of GGTA1. The two ZFEs are called “G1” and “G2”. Together they have the following recognition site: 5′-TCTTATCCCNNNNNNACTGCTGGG-3′ (SEQ ID NO: 26), which falls into the canonical recognition site of 5′-NNT/C NNT/C NNT/C NNN NNN A/GNN A/GNN A/GNN-3′ (SEQ ID NO: 27). The recognition site lies at position 582-606 out of 832 of Exon 9 of GGTA1. These same two ZFEs are used to promote homologous recombination using either the Positive/Negative constructs outlined above or the “Stelloplast”.

The Fok I domain was cloned from Flavobacterium okeanokoites genomic DNA using the primers Forward 5′-GAG GAG GAG GAG CTC GAG GGC GGA GGT ACT AGT CAA CTT GTC AAA AGT GAA CTG GAG G-3′ (SEQ ID NO: 28) and Reverse 5′-CTC CTC CTC CTC GTC GAC GCT TAA TTA AAA GTT TAT CTC GCC GTT ATT AAA TTT CCG-3′ (SEQ ID NO: 29). The resultant PCR product was cloned into pGEM-T Easy (Promega) and sequenced from both ends to ensure that the sequence was perfect. The Fok I domain was then cut out of this vector using Sal I and Xho I and cloned into the Xho I sites of a bacterial and mammalian expression vectors, that will express the protein and add a Poly-Histidine Tag. The Xho I sticky end is at the 5′ terminal of the Fok I endonuclease domain. This can be recut. Ligating a Sal I end to a Xho I end destroys both sites. This is used to check orientation of the Fok I endonuclease domain. These vectors can now be used for any subsequent ZFEs.

The specific vectors used in this case are the bacterial expression vector pRSET A (Invitrogen) that adds a Poly-Histidine Tag to the N-Terminal of the ZFE. Expression of the protein is induced using IPTG using standard methods. The resultant recombinant proteins are purified from the bacterial proteins using the TALON system (Clontech). The mammalian expression system used is a modified version of pcDNA 3.1 from Invitrogen. The modification is simply that a Poly-Histidine fusion tag has been cloned into the multiple cloning site of this vector. Two versions of this vector were made, one containing a Neomycin resistance gene the other Zeocin resistance.

The specific zinc fingers for ZFE G1 and ZFE G2 were constructed using over-lapping PCR with long oligonucleotides, as described above. The resultant PCR produces DNA with the following sequences.

| | |
|---|---|
| Zinc Finger for G1 | |
| GAGCTCGAGCCCGGGGAGAAGCCCTATGCTTGTC | (SEQ ID NO: 30) |
| CGGAATGTGGTAAGTCCTTCAGTCGCAGCGATAA | |
| ACTGGTGCGCCACCAGCGTACCCACACGGGTGAA | |
| AAACCATATAAATGCCCAGAGTGCGGCAAATCTT | |
| TTAGTACCAGCGGCGAACTGGTGCGCCATCAACG | |
| CACTCATACTGGCGAGAAGCCATACAAATGTCCG | |
| GAATGTGGCAAGTCTTTCTCGACCCACCTGGATC | |
| TTATCCGCCACCAACGTACTCACACCGGTACTAG | |
| TTAAGTCGACGAG | |
| Zinc Finger for G2 | |
| CTCGAGCCCGGGGAGAAGCCCTATGCTTGTCCGG | (SEQ ID NO: 31) |
| AATGTGGTAAGTCCTTCAGTCAGCTGGCCCACCT | |
| GCGCGCTCACCAGCGTACCCACACGGGTGAAAAA | |
| CCATATAAATGCCCAGAGTGCGGCAAATCTTTTA | |
| GTCAGAAAAGCTCCCTGATCGCCCATCAACGCAC | |
| TCATACTGGCGAGAAGCCATACAAATGTCCGGAA | |
| TGTGGCAAGTCTTTCTCGCGCAGCGATAAACTGG | |
| TGCGCCACCAACGTACTCACACCGGTACTAGTTA | |
| AGTCGACGAG | |

Both of these fragments of DNA were gel purified and cloned into pGEM-T Easy (Promega). In order to get a perfect sequence is was necessary to sequence at least ten potential plasmid samples (due to the variation in this kind of PCR). However, perfect examples of each sequence were identified. These “zinc fingers” were cut out of pGEM-T easy using Xho I and Sal I and cloned into expression vectors already containing the Fok I endonuclease domain. Orientation of the zinc fingers can be determined as the Xho I sticky end is at the 5′ terminal of the remains can this can be recut. Ligating the Sal I end to a Xho I end destroys both sites.

### Example 9H

### Generation of a GGTA1 Knockout

In order to knock out GGTA1 the following reagents are constructed: two positive/negative DNA homologous recombination DNA constructs (one for each allele), two GGTA1 specific ZFEs and RAD51 protein. The positive negative constructs contain a 7.5 kB fragment of the pig genomic DNA that flanks exon 9 of GGTA1. Exon 9 is the largest coding exon of GGTA1 and its disruption was sufficient to remove all gene function in a mouse model. In one positive/negative construct EGFP under the control of a CMV promotor replaces Exon 9, utilizing Xba I and Sac I sites that flank the Exon. In the other positive/negative construct DsRed2 under the control of a CMV promotor replaces Exon 9, utilizing the same Xba I and Sac I sites that flank the Exon. Both the EGFP and DsRed2 “positive” markers are flanked by Lox P sites. At one end of the construct the coding region for human CD8 alpha chain under the control of a CMV promoter has been added as the “negative” marker. In combination, both ZFEs cut the pig genome only once at a sequence that lies within Exon 9 of GGTA1. Porcine Rad51 was cloned from the pcDNA Yucatan Pig cDNA library and may be used to enhance general recombination.

The cell in which GGTA1 is targeted is a Yucatan Pig embryonic fibroblast. This is the cell type from which most pigs have been cloned by nuclear transfer. For example, (L. Lai, D. Kolber-Simonds, K. W. Park, H. T. Cheong, J. L. Greenstein, G. S. Im, M. Samuel, A. Bonk, A. Rieke, B. N. Day, C. N. Murphy, D. B. Carter, R. J. Hawley, and R. S. Prather. Production of {alpha}-1,3-Galactosyltransferase Knockout Pigs by Nuclear Transfer Cloning. Science, 2002; and GGTA 1 has been knocked out twice in pigs:—Y. Dai, T. D. Vaught, J. Boone, S. H. Chen, C. J. Phelps, S. Ball, J. A. Monahan, P. M. Jobst, K. J. McCreath, A. E. Lamborn, J. L. Cowell-Lucero, K. D. Wells, A. Colman, I. A. Polejaeva, and D. L. Ayares. Targeted disruption of the alpha1,3-galactosyltransferase gene in cloned pigs. Nat.Biotechnol. 20 (3):251-255, 2002).

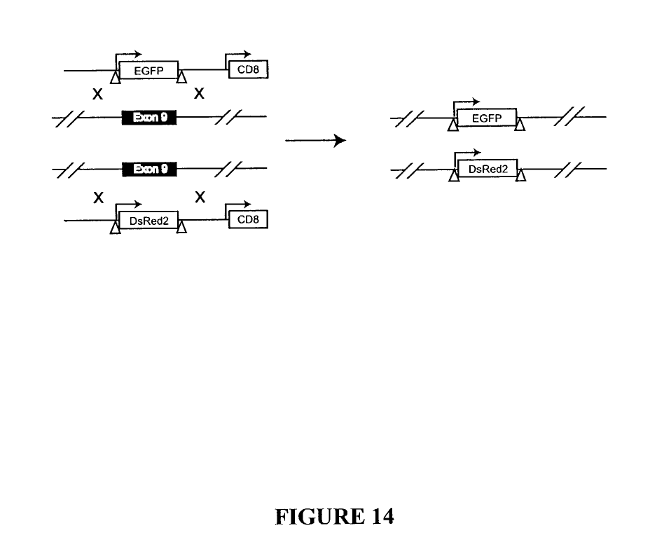

Both alleles of GGTA1 are either knocked out sequentially or simultaneously. Each method will be described in turn. These are illustrated in FIGS. 10 and 11, for example, and described in detail below. Further FIG. 14 provides an illustration of a construct strategy for removing alleles.

The sequential method proceeds in the following way. Firstly, the construct containing EGFP positive marker and CD8 negative marker is introduced into pig embryonic fibroblasts using Fugene 6 (Roche). At the same time the two GGTA1 specific ZFEs and porcine RAD51 are introduced using chemicals like Chariot™ (Active Motif) or BioPorter™ (Gene Therapy Systems, Inc.). After a period of 48 to 72 hours these cells are labeled with an anti-CD8 antibody fluorescently labeled with APC (eBioscience). APC can be detected by FACS analysis as a colour distinct from both EGFP and DsRed2 which are in turn distinct from one another.

By FACS analysis some cells will not produce any color. In these cells there has been no recombination with the introduced DNA. Other cells will produce colour from both EGFP and anti-CD8 APC. In these cells random integration has occurred. The last group of cells will only produce colour from EGFP. In this group of cells it is likely that homologous recombination will have occurred. These cells will be single cell sorted away from the other cells.

Individual EGFP+ cells are cultured in a 96 well tissue culture plate with the appropriate media and feeder cells necessary for viability. The feeder cells will have been previously irradiate so that they cannot divide. Once the wells have divided for a period of one to two weeks there will be between 256 and 65536 cells. Genomic DNA is prepared from half of these cells. PCR is performed to check that EGFP has integrated in the expected position in the genome.

Clones of cells identified in this way are expanded in tissue culture for a further week until there are approximately 5 million cells. A portion of these cells are frozen down at this point. The remaining cells have the construct containing the DsRed2 positive marker and CD8 negative marker introduced using Fugene 6 (Roche). At the same time the two GGTA1 specific ZFEs and porcine RAD51 are again introduced using chemicals like Chariot™ (Active Motif) or BioPorter™ (Gene Therapy Systems, Inc.). After a period of 48 to 72 hours these cells are labeled with an anti-CD8 antibody fluorescently labeled with APC (eBioscience).

By FACS analysis some cells only produce colour from EGFP. In these cells there has been no further recombination with the introduced DNA. Other cells produce color from EGFP, DsRed2 and anti-CD8 APC. In these cells random integration has occurred. The last group of cells produce colour from EGFP and DsRed2 only. In this group of cells, it is likely that homologous recombination will have occurred once more in these cells. These cells will be single cell sorted away from the other cells.

Individual cells which are positive for both are cultured in a 96 well tissue culture plate with the appropriate media and feeder cells necessary for viability. The feeder cells will have been previously irradiated so that they cannot divide. Once the wells have divided for a period of one to two weeks there will be between 256 and 65536 cells. Genomic DNA is prepared from half of these cells. Two PCR reactions are performed to check that both the EGFP and the DsRed2 have integrated in the expected position in the genome. As a further control to check that both alleles of GGTA1 have been knocked out in these cells they are labeled with an anti-Gal3 antibody fluorescently labeled with APC. Cells in which both alleles of GGTA1 have been disrupted produce color from EGFP and DsRed2 but not from the anti-Gal3 APC labeled antibody. A portion of these cells is frozen down at this point.

The remaining cells are expanded in culture for a period of one to two weeks. The Cre recombinase protein will then be introduced using chemicals like Chariot™ (Active Motif) or BioPorter™ (Gene Therapy Systems, Inc.). In a proportion of these cells recombination will occur between the LoxP sites that flank the EGFP and the DsRed2 markers, excising both of these marker genes. These cells are labeled with an anti-Gal3 antibody fluorescently labeled with APC. FACs analysis is used to sort out cells that do not produce any colour from any of EGFP, DsRed2 and APC labeled anti-Gal3. These cells are checked for viability, normal chromosome compliment and any that appear normal are either directly frozen down or used to produce GGTA1 null pigs by nuclear transfer.

The simultaneous removal of both alleles of GGTA1 proceeds in the following way. The embryonic pig fibroblast will have the constructs containing both the EGFP positive marker and the CD8 negative marker as well as ones with the DsRed2 positive marker and CD8 negative marker introduced using Fugene 6 (Roche). At the same time the two GGTA1 specific ZFEs and porcine RAD51 are introduced using chemicals like Chariot™ (Active Motif) or BioPorter™ (Gene Therapy Systems, Inc.). After a period of 48 to 72 hours these cells are labeled with an anti-CD8 antibody fluorescently labeled with APC (eBioscience).

By FACS analysis some cells will only produce no colour. In these cells there has been no recombination with the introduced DNA. Other cells produce colour from EGFP and anti-CD8 APC and/or DsRed and anti-CD8 APC. In these cells random integration has occurred of one or both constructs. The last group of cells produces colour from EGFP and DsRed2 only. In this group of cells, it is likely that homologous recombination has occurred at both alleles. These cells are single cell sorted away from the other cells.

Individual cells which are positive for both are cultured in a 96 well tissue culture plate with the appropriate media and feeder cells necessary for viability. The feeder cells have been previously irradiate so that they cannot divide. Once the wells have divided for a period of one to two weeks there will be between 256 and 65536 cells. Genomic DNA is prepared from half of these cells. Two PCR reactions are performed to check that both the EGFP and the DsRed2 have integrated in the expected position in the genome. As a further control to check that both alleles of GGTA1 have been knocked out in these cells they are labeled with an anti-Gal3 antibody fluorescently labeled with APC. Cells in which both alleles of GGTA1 have been disrupted produce colour from EGFP and DsRed2, but not from the anti-Gal3 APC labeled antibody. A portion of these cells is frozen down at this point.

The remaining cells are expanded in culture for a period of one to two weeks. The Cre recombinase protein is then introduced using chemicals like Chariot™ (Active Motif) or BioPorter™ (Gene Therapy Systems, Inc.). In a proportion of these cells recombination occurs between the LoxP sites that flank the EGFP and the DsRed2 markers, excising both of these marker genes. These cells are labeled with an anti-Gal3 antibody fluorescently labeled with APC. FACs analysis is used to sort out cells that do not produce any colour from any of EGFP, DsRed2 and APC labeled anti-Gal3. These cells are checked for viability, normal chromosome compliment and any that appear normal will either be directly frozen down of used to produce GGTA1 null pigs by nuclear transfer.

### Example 9I

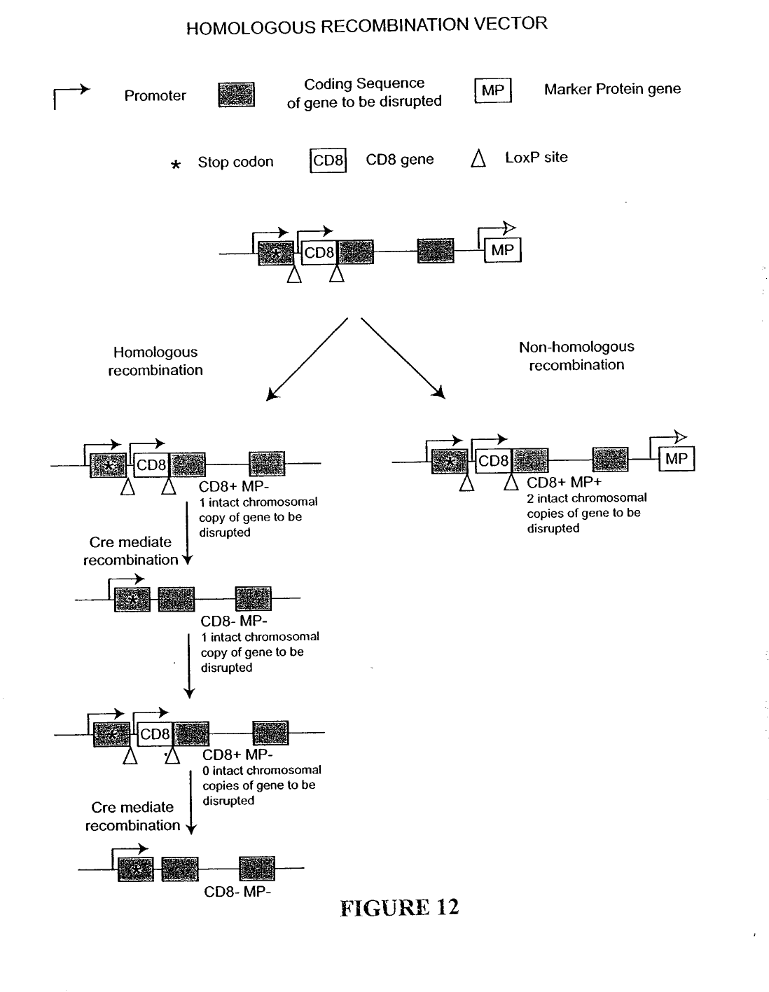

In another embodiment, a stop codon or deletion is introduced into the fragment obtained as described above using conventional techniques such as site directed mutagenesis or enzymatic deletion. The disrupted gene is introduced into a vector suitable for integration into the genome of the donor cells by homologous recombination. Any vector suitable for replacing the chromosomal copies of the gene with the disrupted gene may be used. For example, the disrupted gene may be introduced into the vector illustrated in FIG. 12 or the vector described in Capecchi, 1989, Science 244(4910):1288-1292.

The homologous recombination construct containing the disrupted coding sequence is introduced into the cell using techniques such as lipofection or calcium phosphate transfection. As illustrated in FIG. 12, the homologous recombination vector may comprise a gene encoding a polypeptide harboring an antigenic determinant recognized by the recipient organism which has been disrupted by the creation of a stop codon in the coding sequence. The vector also comprises a promoter operably linked to a nucleic acid encoding CD8 as a reporter gene and a promoter operably linked to a nucleic acid encoding green fluorescent protein (GFP). It will be appreciated that genes encoding detectable products other than CD8 and GFP may also be used in the vector.

As illustrated in FIG. 12, cells in which a homologous recombination event has occurred will be CD8⁺ and GFP⁻, while cells in which the vector has integrated in a random location will be CD8⁺ and GFP⁺. Accordingly, by performing several rounds of FACS separation using commercially available fluorescent antibodies against CD8 and the fluorescence of GFP, cells in which a homologous recombination event has occurred may be separated from cells in which the vector has integrated randomly. The cells in which a homologous recombination event has occurred will contain one disrupted chromosomal copy of the gene encoding a polypeptide harboring an antigenic determinant recognized by the recipient organism (i.e. the gene at which the homologous recombination event has occurred) and one intact chromosomal copy of the gene.

Cre mediated recombination between the LoxP sites is then allowed to occur in the cells in which the disrupted gene has been incorporated into the genome through homologous recombination. Cre may be provided in the cells in which a homologous recombination event has occurred by introducing Cre into the cells through lipofection (Baubonis et al., 1993, Nucleic Acids Res. 21:2025-9, the disclosure of which is incorporated herein by reference in its entirety), or by transfecting the cells with a vector comprising an inducible promoter operably linked to a nucleic acid encoding Cre (Gu et al., 1994, Science 265:103-106, the disclosure of which is incorporated herein by reference in its entirety). Cells in which Cre mediated recombination has occurred will be CD8⁻ and can be separated from CD8⁺ cells in which Cre mediated recombination has not occurred by performing several rounds of FACS.

If desired, the chromosomal structure of the separated cells may be verified by amplifying the target gene using PCR and sequencing the resulting amplicons to confirm the presence of one intact copy of the gene and one disrupted copy. Alternatively, the chromosomal structure of the separated cells may be verified by performing a Southern blot.

The remaining intact copy of the gene encoding a polypeptide harboring an antigenic determinant recognized by the recipient organism is then disrupted as follows. The homologous recombination vector is introduced into the cells comprising one intact copy of the gene and one disrupted copy of the gene. Cells in which homologous recombination has occurred at the formerly intact copy of the gene are identified by separating CD8⁺ GFP⁻ cells from CD8⁺GFP⁺ cells by FACS as described above. In addition, if the cells normally expressed the target gene, fluorescent antibodies against the polypeptide harboring an antigenic determinant recognized by the recipient organism may be used in a FACS procedure to separate cells which do not bind the antibody (i.e. cells in which both copies of the gene have been disrupted) from cells which bind the antibody (i.e. cells in which one copy of the gene is intact). Antibody against the polypeptide harboring an antigenic determinant recognized by the recipient may be obtained as described above.

Another round of Cre mediated recombination is allowed to occur to delete the CD8 gene in the cells.

If desired, the chromosomal structure of the separated cells may be verified by amplifying the target gene using PCR and sequencing the resulting amplicons to confirm the presence of two disrupted copies of the target gene. Alternatively, the chromosomal structure of the separated cells may be verified by performing a Southern blot.

FIG. 12 summarizes the above procedures.

The above procedure is repeated for each gene in which it is desired to generate a disruption such that the desired genes encoding polypeptides harboring antigenic determinants recognized by the recipient organism are sequentially disrupted, resulting in cells which have a desired set genes disrupted.

Alternatively, cells in which both chromosomal copies of a gene harboring an antigenic determinant recognized by a recipient organism may be obtained as follows. The first chromosomal copy of the target gene is disrupted as described above. As described above, a first homologous recombination vector comprising a gene encoding a polypeptide harboring an antigenic determinant recognized by the recipient organism which has been disrupted by the creation of a stop codon in the coding sequence is introduced into the cell. The vector also comprises a promoter operably linked to a nucleic acid encoding CD8 as a reporter gene and a promoter operably linked to a nucleic acid encoding green fluorescent protein (GFP). It will be appreciated that genes encoding detectable products other than CD8 and GFP may also be used in the vector.

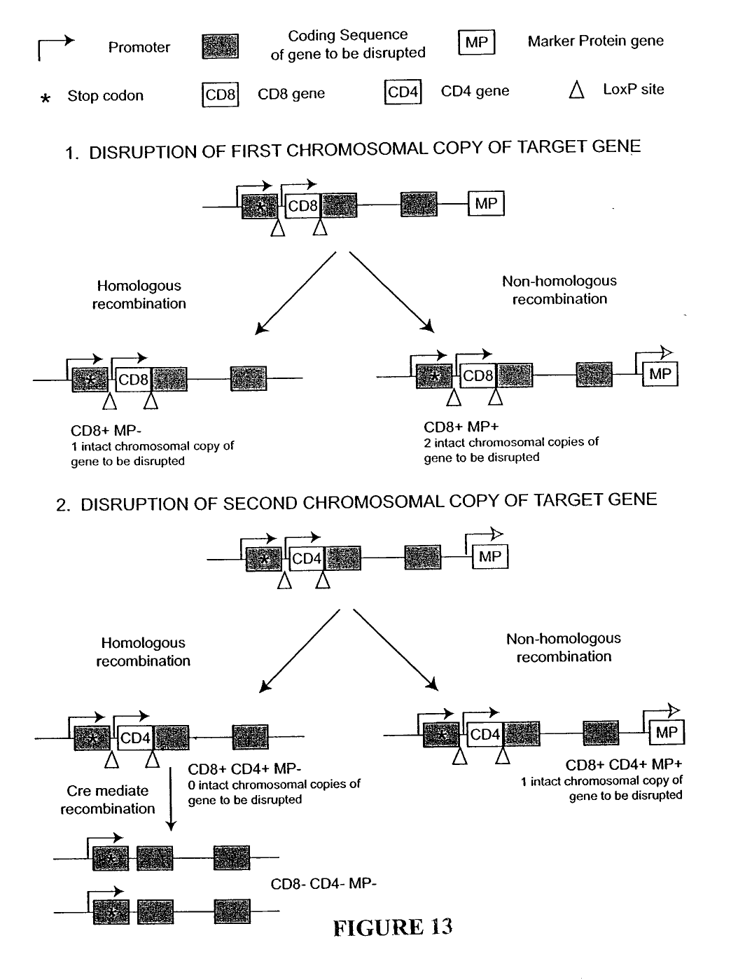

As illustrated in FIG. 13, cells in which a homologous recombination event has occurred will be CD8⁺ and GFP⁻, while cells in which the vector has integrated in a random location will be CD8⁺ and GFP⁺. Accordingly, by performing several rounds of FACS separation using commercially available fluorescent antibodies against CD8 and the fluorescence of GFP, cells in which a homologous recombination event has occurred may be separated from cells in which the vector has integrated randomly. As illustrated in FIG. 13, the cells in which a homologous recombination event has occurred will contain one disrupted chromosomal copy of the gene encoding a polypeptide harboring an antigenic determinant recognized by the recipient organism (i.e. the gene at which the homologous recombination event has occurred) and one intact chromosomal copy of the gene.

If desired, the chromosomal structure of the separated cells may be verified by amplifying the target gene using PCR and sequencing the resulting amplicons to confirm the presence of one intact copy of the gene and one disrupted copy. Alternatively, the chromosomal structure of the separated cells may be verified by performing a Southern blot.

A second homologous recombination vector is then introduced into the cells in which one chromosomal copy of the target gene has been disrupted. The second homologous recombination vector is similar to the one used to disrupt the first chromosomal copy of the target gene except that rather than containing a gene encoding the CD8 protein operably linked to a promoter, the second homologous recombination vector contains a gene encoding the CD4 protein operably linked to a promoter. It will be appreciated that genes encoding detectable products other than CD4 may also be used in the vector. As illustrated in FIG. 13, cells in which the second homologous recombination vector has integrated into the chromosome through a homologous recombination event occurred will be CD4⁺ and GFP⁻, while cells in which the vector has integrated in a random location will be CD4⁺ and GFP⁺. Accordingly, by performing several rounds of FACS separation using commercially available fluorescent antibodies against CD4 and the fluorescence of GFP, cells in which a homologous recombination event has occurred may be separated from cells in which the vector has integrated randomly. If desired, the FACS analysis may also use antibody against CD8, since the cells will be CD8⁺ by virtue of the chromosomal integration of the first homologous recombination vector through a homologous recombination event. In addition, if the cells normally expressed the target gene, fluorescent antibodies against the polypeptide harboring an antigenic determinant recognized by the recipient organism may be used in a FACS procedure to separate cells which do not bind the antibody (i.e. cells in which both copies of the gene have been disrupted) from cells which bind the antibody (i.e. cells in which one copy of the gene is intact). Antibody against the polypeptide harboring an antigenic determinant recognized by the recipient may be obtained as described above. As illustrated in FIG. 13, the cells in which the second homologous recombination event has occurred at the second chromosomal copy of the target gene will have both chromosomal copies of the target gene disrupted.

Cre mediated recombination between the LoxP sites is then allowed to occur in the cells in which the both chromosomal copies of the target gene have been disrupted Cells in which Cre mediated recombination has occurred in both of the integrated vectors will be CD8⁻ and CD4⁻ and can be separated from cells in which Cre mediated recombination has not occurred in both of the integrated vectors (which will be CD8⁺CD4⁺, CD8⁺CD4⁻, or CD8⁻CD4⁺ depending on whether Cre mediated recombination has not occurred at all or whether it occurred in one of the two integrated vectors) by performing several rounds of FACS. In addition, if the cells normally expressed the target gene, fluorescent antibodies against the polypeptide harboring an antigenic determinant recognized by the recipient organism may be used in a FACS procedure to separate cells which do not bind the antibody (i.e. cells in which both copies of the gene have been disrupted) from cells which bind the antibody (i.e. cells in which one copy of the gene is intact). Antibody against the polypeptide harboring an antigenic determinant recognized by the recipient may be obtained as described above.

If desired, the chromosomal structure of the separated cells may be verified by amplifying the target gene using PCR and sequencing the resulting amplicons to confirm the presence of two disrupted copies of the target gene. Alternatively, the chromosomal structure of the separated cells may be verified by performing a Southern blot.

FIG. 13 summarizes the above procedures.

The above procedure is repeated for each gene in which it is desired to generate a disruption such that the desired genes encoding polypeptides harboring antigenic determinants recognized by the recipient organism are sequentially disrupted, resulting in cells which have a desired set genes disrupted.

It will be appreciated that, if desired, the homologous recombination vector used to disrupt the first chromosomal copy of the target gene may be the vector which contains the CD4 gene and the homologous recombination vector used to disrupt the second chromosomal copy of the target gene may be the vector which contains the CD8 gene.

It will be appreciated that other methodologies for generating donor organisms in which one or more genes encoding a polypeptide harboring an antigenic determinant recognized by the recipient organism has been disrupted may also be employed.

If desired, the structure of the targeted genes in the cells obtained by FACS analysis may be evaluated by performing a Southern blot or PCR analysis to confirm that the both copies of the targeted genes have been disrupted.

Alternatively, genes encoding polypeptides comprising an antigenic determinant recognized by a desired recipient organism may be disrupted using a positive/negative construct, a gene trapping construct, an overlapping knockout construct, or a construct which inserts a stop codon in all three reading frames after homologous recombination.

### EXAMPLE 10

### Analysis of Cells Containing Disrupted Genes

To test the antigenicity of cells where one or several genes have been removed. Primary cells or cell lines are subject to antibody and complement incubation to test to what extent these two components lyse the genetically altered cells. (Taniguchi S., Neethling F. A., Korchagina E. Y., Bovin N., Te Y., Kobayashi T., Niekrasz, Li S., Koren E., Oriol R., and Cooper D. K. In vivo immunoadsorption of anti-pig antibodies in baboons using a specific Gal(alpha)1-3Gal column) Transplantation. Nov. 27, 1996 ;62(10):1379-84). In brief cells are cultured, for example, on Terasaki trays or the equivalent and then exposed to heat inactivated human serum, and after this, exposed to complement followed by incubation with calcein and ethidium homodimer. Subsequently, the cells are visualized by an epiflourescent microscope to evaluate if cells with a genetic modification are less susceptible to complement and serum induced lysis than unmodified cells.

It is valuable to test serum from a mix of different individuals, different ages, different sexes and races, but also from people who have been exposed to pig tissue, for example individuals who have been subject to hemoperfusion using pig hepatpcytes. Further, it is useful to compare if patent serum change antibody profile after exposure to pig tissue. Also, serum from patients exposed to pig tissue may be used.

Further, if desired, tissue culture cells or cells from transgenic or genetically modified animals in which one or more genes encoding polypeptides harboring an antigenic determinant recognized by the recipient organism have been disrupted may be evaluated to determine the extent to which they express polypeptides recognized sera from the recipient organism. After each such gene has been disrupted as described above, the tissue culture cells or cells from a transgenic or genetically modified animal are contacted with sera from the recipient organism and the extent to which the cells are recognized by antibodies in the sera is determined by FACS and/or ELISA analysis.

If desired, the extent to which the tissue culture cells or cells from a genetically modified animal in which one or more genes encoding polypeptides harboring an antigenic determinant recognized by the recipient organism has been disrupted are recognized by sera from the recipient organism may be evaluated after each disruption by injecting the cells into the desired recipient organism or into suitable test animals as follows. A large number of cells, for example 10⁶ cells, are injected subcutaneously in an animal, such as a non-human primate, mammal or the recipient organism. If desired, the non-human primate, mammal, or recipient organism may also be treated with an immunosuppressive agent. The animals are observed for one month to look for any sign of rejection. If the cells an undesirable level of rejection occurs, more genes encoding polypeptides harboring an antigenic determinant recognized by the recipient organism are disrupted.

If the desired level of rejection or no signs of rejection are observed, the animals are sacrificed, and skin and tissues near the site of injection are analyzed by histochemistry. Tissue or cells are obtained from the animals, frozen, and sectioned with a cryostat. The sections are stained with detectable antibodies against the polypeptides encoded by the genes which were disrupted to determine whether the polypeptides are present.

Alternatively, the cells may be labeled with CFSE prior to injection (Oehen, et al., 1997, J. Immunol. Methods 207(1):33-42, the disclosure of which is incorporated herein by reference in its entirety), a fluorescent dye, which makes it easy to detect the injected cells in the tissues to confirm that the cells are still viable, yet not rejected.

### EXAMPLE 11

### Generation of Animals Comprising Cells with Disrupted Genes

After donor cells in which a desired number of genes encoding polypeptides harboring antigenic determinants recognized by the recipient organism have been disrupted are generated as described above, they are used to generate genetically modified animals for use as organ donors. Nuclear transfer using nuclei from cells having a desired number of genes encoding polypeptides harboring antigenic determinants recognized by the recipient organism disrupted is performed as described by Wilmut et al., 1997, Nature. 385(6619)810-813, U.S. Pat. No. 6,147,276, U.S. Pat. No. 5,945,577 or U.S. Pat. No. 6,077,710, the disclosures of which are incorporated herein by reference in their entireties Briefly, the nuclei are transferred into enucleated fertilized oocytes. A large number of oocytes are generated in this manner. Approximately ten animals are fertilized with the oocytes, with at least six fertilized embryos being implanted into each animal and allowed to progress through birth.

Another technique to generate genetically modified animals without using nuclear transfer is co-injection of sperm and the components for homologous recombination (DNA with or without protein) into oocytes. (Perry, A. C. F. et al. Nat Biotechnol. November 2001;19(11):1071-3 “Efficient metaphase II transgenesis with different transgene archetypes,” the disclosure of which is hereby incorporated by reference in its entirety.). This technique can generate genetically modified animals without using nuclear transfer, and thereby, bypasses many of the side effects associated with nuclear transfer.

The isolation and culture of metaphase II oocytes for microinjection have essentially been described (Kimura, Y. et al. Biol Reprod. April 1995;52(4):709-20 Intracytoplasmic sperm injection in the mouse and Chatot, C. L. et al. March 1990; 42(3):432-40 Development of 1-cell embryos from different strains of mice in CZB medium. Biol Reprod). Microinjection is performed 14-20 h after administration of human chorionic gonadotropin. Spermatozoa is isolated by finely chopping two acutely isolated caudae epidydimis essentially as described (Perry, A. C. F. Science. May 14, 1999 ;284(5417):1180-3 Mammalian transgenesis by intracytoplasmic sperm injection) at room temperature. The sperm suspension is filtered through tissue paper and adjusted to correct volume for freeze-thaw or for Triton X-100 extraction procedure.

Sperm preparation and mixing of DNA and protein components. The sperm is resuspended in nuclear isolation media (Kuretake, S. et al. Biol Reprod. October 1996 ;55(4):789-95 Fertilization and development of mouse oocytes injection with isolated sperm heads) and is either subject to freeze-thawing or extraction with Triton X-100 before mixing with the DNA with or without protein components. Freeze-thawing is essentially prepared as described (Perry, A. C. F. Science. 1999 ibid, Kuretake, S. ibid and Wakayama, T. et al. Nat Biotechnol. July 1998;16(7):639-41 Development of normal mice from oocytes injected with freeze-dried spermatozoa.) by freezing to −80° C. in aliquots and then rapidly thawing immediately before mixing with DNA and protein components. Sperm for Triton extraction is prepared by adding Triton X-100 to a final concentration of 0.05% (vol/vol) in the sperm suspension and mixing by triturating for 1 minute at 25° C. or 2° C. before two washes in nuclear isolation media with or without Triton X-100, at 2° C., sperm is resuspended in the same buffer. The mixture of sperm, either prepared by freeze-thawing or by Triton X-100 and DNA with or without protein components is triturated for 1 minute before mixing with polyvinylpyrrolidone (PVP; average Mr 360 000) solution to give a final concentration of 10% (wt/vol) PVP and is then placed on the microscope stage for injection.

With regard to gamete microsurgery, embryo culture and transfer, all microinjections are performed in HEPES-buffered CZB medium (Charcot, C. L. ibid) at room temperature within 90 minutes of the mixing of sperm, DNA with or without protein components. Microinjection is performed by piezo actuation of a blunt-ended pipette-tip (internal diameter, 5 μM) using a prime Tech PMM-150 FU piezo impact unit essentially as described (Perry, A. C. F. Science. Ibid). Sperm heads that have undergone decapitation during preparation are used for microinjection. Approximately 5-10 minutes after microinjection oocytes are transferred to droplets of KSOM (Speciality Media, Phillisburg, N.J.) under mineral oil equilibrated in 5% (vol/vol in air) CO₂ at 37° C. and cultured until embryo transfer. When appropriate, embryos are examined after 3.5 days by epifluorescence microscopy for expression of GFP or other marker, using a UV light source (480 nm) with fluorescein isothiocyanate filters. Full-term development, one- to two-cell embryos or morulac/blastocysts were respectively transferred to the oviducts or uteri of foster mothers. In the case when screening by fluorescent marker is not possible, a PCR based screen can be performed. When an efficiency rate of homologous recombination above 10% has been achieved, remaining embryos are subject to implantation.

Animals comprising cells, organs or tissues in which a desired number of genes encoding polypeptides harboring an antigenic determinant recognized by the recipient organism have been disrupted may also be generated using other methods familiar to those skilled in the art. For example, as discussed above, stem cell-based technologies may be employed.

Organs from the animals born by the fertilized animals are used for xenotransplantation experiments as follows. Small pieces of the organs desired to be used in xenotransplantation, including but not limited to liver, pancreas, kidney, heart, heart valve, lung, intestine, cornea and/or endothelial tissue from the big vessels are placed under the kidney capsule of non-human primates, the recipient organism, or other suitable animal models. The animals are observed for two months to look for signs of rejection.

If the level of rejection observed is less than desired, additional genes encoding polypeptides harboring an antigenic determinant may be disrupted until a desired level of rejection is obtained. If the level of rejection observed is still less than desired, intracellular proteins harboring antigenic determinants recognized by the recipient organism may be identified as described above and the genes encoding them may be sequentially disrupted until a desired level of rejection is obtained.

Further, If the level of rejection observed is less than desired, then the MHC Class I and/or Class II genes of the donor organism to be used in xenotransplantation may be replaced by the counterpart gene from the recipient organism to further reduce the level of rejection and to determine the extent to which they are tolerated, as described herein. If the cells are to be tested in an animal model other than recipient organism, the MHC Class I and Class II genes may be replaced by their counterparts from the test animal and then replaced by their counterparts from the recipient organism prior to use in the recipient organism. Preferably, the peptide binding partners recognized by the MHC Class I and Class II genes are also provided. Replacement of the MHC Class I or Class II genes may be accomplished by using homologous recombination to replace the nucleic acid sequence encoding the MHC Class I or Class II proteins from the donor organism with the nucleic acid sequence encoding the MHC Class I or Class II proteins from the recipient organism or test organism using methods such as those described in (Ignatowicz et al., 1996, Cell. 84(4):521-529), the disclosure of which is incorporated herein by reference in its entirety). In addition, the nucleic acid sequence encoding the MHC Class I or Class II may be fused in frame to a nucleic acid sequence encoding the peptide binding partner recognized by the MHC Class I or Class II proteins, so that the peptide binding pockets of the MHC Class I or Class II proteins are occupied by their cognate peptides in the genetically modified animals. If desired, a nucleic acid sequence encoding a spacer peptide may be disposed between the nucleic acid encoding the MHC Class I or Class II protein and the nucleic acid encoding the cognate peptide.

When a desired level of rejection is observed, entire tissues or organs may be transplanted into non-human primates, the recipient organism, or suitable animal models to determine the extent to which they are tolerated. Animals are observed for six months to determine the extent of tolerance. If desired, the animals may be given immunosuppressants, such as cyclosporin.

### EXAMPLE 12

### Use of Tissues or Organs for Xenotransplantation in Humans or other Recipient Organisms

Tissues or organs are obtained from donor organisms in which one or more genes encoding a polypeptide comprising an antigenic determinant recognized by sera from the recipient organism have been disrupted. The tissues or organs are transplanted into the human or other recipient organism in need thereof using conventional surgical procedures. If desired or necessary, immunosuppressants may be administered to further reduce the likelihood of rejection.

### EXAMPLE 13

### Implantation of Tissues or Organs on a Scaffold

Donor cells having the desired genes disrupted may are seeded on a scaffold which forms the support for the tissue or organ. The scaffold may be a synthetic polymer or may have a biological component, such as a collagen. Donor cells having the desired genes disrupted are grown on the scaffold. The scaffold comprising the donor cells is then implanted into the recipient organism using standard surgical procedures.

### EXAMPLE 14

### Administration of Cells Having Desired Genes Disrupted to a Recipient Organism

In some embodiments of the present invention, donor cells which are not associated with one another to form a tissue or organ and which have disruptions in one or more genes encoding polypeptides comprising antigenic determinants recognized by the recipient organism are administered to the recipient organism. The donor cells may be obtained from organisms generated as described above. Alternatively, the donor cells may be tissue culture cells or primary cultured cells.

The donor cells may be any type of cell which provides a beneficial factor to the recipient organism. For example, in one embodiment, the donor cells may be brain cells or fetal brain cells which provide dopamine to a recipient suffering from Parkinson's disease after implantation into the brain of the recipient organism. In another embodiment, the donor cells may be brain cells or fetal brain cells which provide a factor which inhibits the formation of amyloid plaques in a recipient suffering from Alzheimer's disease.

In one embodiment, pluripotent stem cells may be obtained from a genetically modified organism generated as described above. The pluripotent stem cells are allowed to differentiate into a desired cell type. For example, the stem cells may be allowed to differentiate into muscle cells, such as heart muscle cells, bone cells, islet cells, skin cells, nerve cells, endothelial cells or any other cell type which would provide a beneficial effect after introduction into the recipient organism. For example, the recipient may be suffering from a spinal cord injury, stroke, burns, heart disease, osteoarthritis, rheumatoid arthritis, or diabetes.

In one embodiment, heart muscle cells prepared in accordance with the present invention may be transplanted into a recipient suffering from heart disease. In another embodiment, donor islet cells prepared in accordance with the present invention may be introduced into a recipient suffering from diabetes.

In another embodiment, donor cells in which one or more genes encoding polypeptides comprising antigenic determinants recognized by the recipient organism have been disrupted may be genetically engineered to express a factor beneficial to the recipient organism. In such embodiments, a vector encoding the beneficial factor is introduced into the donor cells. For example, the vector may encode a growth factor or cytokine. In other embodiments, the vector may encode a polypeptide whose absence or production at insufficient levels has caused a disease in the recipient organism. In another embodiment, the vector may encode a factor which inhibits the activity or reduces the amount of a nucleic acid or polypeptide whose production at abnormally high levels has caused a disease in the recipient organism.

Donor cells prepared as described above may be administered to the recipient organism in any manner consistent with their intended use. For example, the cells may be introduced into the recipient organism by injection, intravenous administration, grafting, transplantation, or any other means familiar to those skilled in the art.

### EXAMPLE 15

### Gene Therapy Using Genetically Modified Animals, Resistant to Rejection as Vectors

Any of the animals of the invention and those described herein can be engineered to include a gene encoding a “substance” that can be secreted from either a particular cell type or organ, depending on what promoter is used. In such methods, a vector encoding the desired substance is introduced into the cells to be used to generate the animal using techniques familiar to those skilled in the art. The produced “substance ” is a source for therapy when a piece of an organ, entire organ or cells from the genetically modified animal will be transplanted into the individual needing this substance. A piece of tissue can, for example, be placed under the skin for easy access, if desired it can be removed at any time. If only temporary or intermittent treatment is desired the “substance” can then be expressed under an inducible promoter, an example would be tetracycline. The level of induction of the “substance” would then be regulated by tetracycline supplement in the diet.

Examples of secreted “substances” that can be used are hormones, growth factors interleukins, neuropeptides, antibodies or any protein, lipid or carbohydrate that can have a medicinal effect either at the cell surface of other cells or intracellularly, if internalized by the target cell. The effect can either be stimulatory or inhibitory.

Some specific examples include growth hormone, for example for growth hormone deficient children; Erythropoetin (EPO), anemic conditions; insulin: islets or the pancreas can be transplanted into diabetic patients; tumor necrosis factor α (TNF-alpha) antibodies, for example, for inflammatory diseases such as rheumatoid arthritis and Crohn's disease; antibodies against protein products encoded by oncogenes such as C-erbB-2, for example used for breast cancer and other cancers; anti-CD4 antibodies for example, for rheumatoid arthritis or psoriasis; anti-human Epidermal Growth Factor Receptor type 2 antibodies, for example, for breast cancer and other cancers; anti-Interleukin antibodies, such as anti-IL-1, anti-IL-8, anti IL-10, anti-IL-12 and anti-IL-15 to be used, for example, in inflammatory diseases, such as autoimmune diseases, rheumatoid arthritis, psoriasis, inflammatory bowel disease and in cancerous disease; anti-Interleukin 15 receptor anti-bodies for use against lymphoma and other malignancies, for example; anti-CD20 antibodies to be used, for example, for hemolytic anemia in autoimmune diseases and other hematopoetic disorders such as leukemia and lymphomas; anti-isotypic IGE antibodies for allergy; anti-LG914 antibodies for arteriosclerosis; Interferon-α for chronic hepatitis C, hairy cell leukemia and AIDS-related Kaposi's sarcoma and chronic myelogenous leukemia (CML), for example; Interferon-γ for multiple sclerosis; granulocyte-macrophage colony-stimulating factor (GM-CSF) for malignancies; Tissue Factor for hematolytic abnormalities in general and in leukemia and in liver disease causing he disorders; hyaluronic acid cells producing hylauronic acid can be implanted into joints in patients suffering from pain in osteoarthritis; transgene expression in donor animal such as pig of proteins, lipids and carbohydrates to induce tolerance in xenotransplantation; anti-CD40, CD28, CD25 and IL-2 antibodies and OKT3; anti-idiotypic antibodies against naturally formed antibodies; anti-isotypic IgG, IgM and IgA antibodies. The preceding is a non exclusive list of some exemplary gene therapy applications.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. All documents cited herein are incorporated herein by reference in their entirety.

What is claimed is:

1. A genetically engineered cell in which at least one gene encoding a polypeptide comprising an antigenic determinant which is recognized by a desired recipient organism or at least one gene which encodes a protein associated with the synthesis of a molecule comprising an antigenic determinant recognized by the desired recipient organism has been disrupted.

2. The genetically engineered cell of claim 1, wherein both chromosomal copies of said at least one gene have been disrupted.

3. The genetically engineered cell of claim 1, wherein at least one gene encoding a polypeptide comprising an antigenic determinant which is recognized by human beings has been disrupted.

4. The genetically engineered cell of claim 2, wherein a plurality of genes encoding polypeptides comprising antigenic determinants recognized by a desired recipient organism have been disrupted.

5. The genetically engineered cell of claim 2, wherein at least two, at least 4, at least 5, at least 10, at least 15, at least 20, at least 25, at least 35, at least 40 or than 40 genes encoding polypeptides comprising antigenic determinants recognized by the recipient organism have been disrupted.

6. The genetically engineered cell of claim 2, wherein substantially all of the genes encoding polypeptides comprising antigenic determinants recognized by the recipient organism have been disrupted.

7. The genetically engineered cell of claim 2, wherein said cell is from an organism selected from the group consisting of a mammal, a marsupial, a teleost fish, and an avian.

8. The genetically engineered cell of claim 7, wherein said mammal is selected from the group consisting of a non-human primate, a sheep, a goat, and a cow.

9. The genetically engineered cell of claim 7, wherein said avian is a chicken.

10. The genetically engineered cell of claim 7, wherein said cell is from a pig.

11. The genetically engineered cell of claim 10, wherein said cell is selected from the group consisting of primary pig skin fibroblasts, pig granulosa cells, pig stem cells, pig germ cells, pig peripheral blood cells, pig hematopoetic stem cells and primary pig fetal fibroblasts.

12. The genetically engineered cell of claim 1, wherein said at least one gene has been disrupted by replacing at least one chromosomal copy of said gene with a homologous sequence comprising a stop codon in the open reading frame which encodes said polypeptide.

13. The genetically engineered cell of claim 1, wherein said at least one gene has been disrupted by replacing at least one chromosomal copy of said gene with a homologous sequence comprising a stop codon in all three reading frames.

14. The genetically engineered cell of claim 1, wherein said at least one gene has been disrupted by replacing at least one chromosomal copy of said gene with a homologous sequence comprising a deletion.

15. The genetically engineered cell of claim 1, wherein said at least one gene has been disrupted by replacing at least one chromosomal copy of said gene with a non-homologous replacement nucleotide sequence flanked by nucleotide sequences homologous to a genomic sequence in which homologous recombination is desired.

16. The genetically engineered cell of claim 15, wherein said replacement nucleotide sequence comprises a gene encoding a marker or a gene encoding a polypeptide from said desired recipient organism.

17. The genetically engineered cell of claim 16, wherein said gene encoding a polypeptide from said desired recipient organism comprises a gene encoding a major histocompatability complex (MHC) Protein.

18. The genetically engineered cell of claim 2, wherein said desired recipient organism is a human being.

19. The genetically engineered cell of claim 2, wherein said at least one gene is a gene other than the GGTA1 gene.

20. The genetically engineered cell of claim 2, wherein said gene encodes a polypeptide that includes an antigenic determinant or a polypeptide associated with the synthesis or modification of an antigenic determinant.

21. The genetically engineered cell of claim 1, wherein said antigenic determinant comprises a polypeptide, a carbohydrate, or a lipid.

22. A recombinant nucleic acid comprising a 5′ region homologous to a portion of a gene responsible for the production of an antigenic determinant recognized by a desired recipient organism or a 5′ region homologous to a portion of a gene encoding a polypeptide associated with the synthesis of a molecule comprising an antigenic determinant recognized by said desired organism, a 3′ region homologous to a portion of a gene responsible for the production of an antigenic determinant recognized by said desired recipient organism or a 3′ region homologous to a portion of a gene encoding a polypeptide associated with the synthesis of a molecule comprising an antigenic determinant recognized by said desired organism, and a nucleotide sequence which prevents the synthesis of an antigenic determinant recognized by said desired recipient organism, said nucleotide sequence being disposed between said 5′ region and said 3′ region.

23. The recombinant nucleic acid of claim 22, wherein at least a portion of said nucleotide sequence which prevents the synthesis of an antigenic determinant recognized by said desired recipient organism is disposed between said 5′ region and said 3′ region, said at least a portion containing an alteration therein which prevents the synthesis of an antigenic determinant recognized by said desired recipient organism.

24. The recombinant nucleic acid sequence of claim 22, wherein said alteration comprises at least one deletion.

25. The recombinant nucleic acid sequence of claim 22, wherein said alteration comprises a stop codon in the open reading frame which encodes a polypeptide comprising an antigenic determinant recognized by said desired recipient organism.

26. The recombinant nucleic acid sequence of claim 22, wherein said alteration comprises a nucleotide sequence containing a stop codon in all three reading frames.

27. The recombinant nucleic acid sequence of claim 22, wherein said alteration comprises a replacement sequence comprising a gene encoding a marker or a gene encoding a polypeptide from said desired recipient organism.

28. The recombinant nucleic acid sequence of claim 27, wherein said gene encoding a polypeptide from said desired recipient organism comprises a gene encoding an MHC Protein.

29. The recombinant nucleic acid sequence of claim 22, wherein said nucleotide sequence which prevents the synthesis of an antigenic determinant recognized by said desired recipient organism comprises a positive marker indicative of integration somewhere in the genome and a negative marker indicative of random integration in the genome.

30. The recombinant nucleic acid sequence of claim 29, wherein said positive marker is flanked by nucleotide sequences homologous to the genomic region in which integration via homologous recombination is desired.

31. The recombinant nucleic acid sequence of claim 22, wherein said nucleotide sequence which prevents the synthesis of an antigenic determinant recognized by said desired recipient organism comprises a promoterless marker gene flanked by nucleotide sequences which will put said marker gene under the control of the promoter which directs transcription of said gene encoding a polypeptide comprising an antigenic determent recognized by a desired recipient organism if homologous recombination occurs.

32. The recombinant nucleic acid sequence of claim 22, wherein said nucleotide sequence which prevents the synthesis of an antigenic determinant recognized by said desired recipient organism comprises a portion of a gene encoding a nonfunctional portion of a marker protein, said portion of said gene encoding a nonfunctional portion of a marker protein being flanked by nucleotide sequences homologous to the desired integration site.

33. The recombinant nucleic acid sequence of claim 22, further comprising at least one nucleic acid encoding a detectable polypeptide, said at least one nucleic acid being operably linked to a promoter.

34. The recombinant nucleic acid sequence of claim 33, wherein said recombinant nucleic acid comprises a nucleic acid encoding CD8 operably linked to a promoter and a nucleic acid encoding green fluorescent protein operably linked to a promoter.

35. The recombinant nucleic acid sequence of claim 33, wherein said detectable polypeptide is selected from the group consisting of CD8, green fluorescent protein (GFP), Red fluorescent protein, Flag tag, HA tag, c-myc, GST, mbp, and polyhistidine.

36. The recombinant nucleic acid sequence of claim 33, wherein at least one nucleic acid encoding a detectable polypeptide is flanked by a site which enables excision of said nucleic acid encoding a detectable polypeptide.

37. The recombinant nucleic acid sequence of claim 33, wherein said site which enables subsequent removal of a non-homologous sequence is a Lox P site or an Frt site.

38. The recombinant nucleic acid sequence of claim 22, wherein said gene responsible for the production of an antigenic determinant is a gene other than the GGTA1 gene.

39. The recombinant nucleic acid sequence of claim 22, wherein said gene is responsible for the production of an antigenic determinant which may be a polypeptide, a carbohydrate or a lipid, or which results from the modification of a polypeptide, carbohydrate or lipid.

40. A method of disrupting a gene encodes a polypeptide responsible for the production of an antigenic determinant recognized by a desired recipient organism comprising:
introducing a nucleic acid comprising a sequence homologous to at least a portion of the coding region of said gene into a cell, wherein said homologous sequence comprises a disruption in said coding region which prevents said cell from expressing the full length polypeptide normally encoded by said coding region; and
replacing at least one chromosomal copy of said gene with said homologous sequence comprising said disruption in said coding region.

41. The method of claim 40, further comprising enhancing the rate of recombination by introducing a double stranded break in the nucleic acid in a region in the vicinity of the gene encoding a polypeptide comprising the antigenic determinant.

42. The method of claim 41, wherein said double stranded break is introduced using at least one zinc finger endonuclease domain.

43. The method of claim 40, wherein said disruption in said coding region comprises at least one stop codon in one open reading frame encoding said polypeptide.

44. The method of claim 43, wherein said disruption comprises a nucleotide sequence containing a stop codon in all three reading frames.

45. The method of claim 40, wherein said gene which encodes a polypeptide comprising an antigenic determinant recognized by a desired recipient organism is a gene other than the GGTA1 gene.

46. A method of identifying an antigenic determinant from a donor organism which is recognized by a recipient organism comprising:
obtaining a screening composition comprising a plurality of molecules from said donor organism;
contacting said plurality of molecules with naturally occurring immunoglobulin family proteins; and
identifying an antigenic determinant that is detected by said naturally occurring immunoglobulin family proteins.

47. The method of claim 46, wherein said screening composition comprises a plurality of molecules isolated from the surface of cells from said donor organism.

48. The method of claim 46, wherein said molecule is selected from the group consisting of a polypeptide, a lipid, a carbohydrate, and a molecule comprising any combination of the foregoing molecules.

49. The method of claim 46, wherein said naturally occurring immunoglobulin family proteins comprise immune sera from said recipient organism.

50. The method of claim 45, wherein said naturally occurring immunoglobulin family proteins comprise a polyclonal immunoglobulin population derived from said recipient organism.

51. A method of identifying a gene responsible for the production of an antigenic determinant from a donor organism that is recognized by a recipient organism comprising:
obtaining a plurality of nucleic acids encoding a plurality polypeptides from said donor organism and expressing the plurality of polypeptides;
contacting said plurality of polypeptides with naturally occurring immunoglobulin family proteins present on the surface of or obtained from natural killer cells or T cells from the recipient organism; and
identifying cells recognized by said naturally occurring immunoglobulin family proteins, whereby said cells comprise a gene from a donor organism which encodes a polypeptide comprising an antigenic determinant recognized by said recipient organism or a gene from said donor organism which encodes a polypeptide associated with the synthesis of a molecule comprising an antigenic determinant recognized by said recipient organism.

52. A method of identifying a gene from a donor organism which encodes a polypeptide comprising an antigenic determinant recognized by a recipient organism or a gene from said donor organism which encodes a polypeptide associated with the synthesis of a molecule comprising an antigenic determinant recognized by said recipient organism comprising:
obtaining a cDNA library comprising a plurality of genes encoding polypeptides from said donor organism;
expressing said polypeptides in host cells;
contacting said host cells with naturally occurring immunoglobulin family proteins which detect antigenic determinants recognized by said recipient organism; and
identifying a host cell which expresses a polypeptide recognized by said naturally occurring Immunoglobulin family proteins.

53. The method of claim 52, wherein said naturally occurring immunoglobulin family proteins derived from said recipient organism comprises immune sera, wherein said expressed polypeptide is recognized an antibody in said immune sera.

54. The method of claim 52, wherein said naturally occurring immunoglobulin family proteins derived from said recipient organism comprise a polyclonal antibody population.

55. The method of claim 52, wherein said naturally occurring immunoglobulin family proteins comprise molecules present on the surface of or obtained from naturaly killer cells or T cells from the recipient organism.
